(19) United States
(12) Patent Application Publication
(10) Pub. No.: US 2025/0340942 A1
(43) Pub. Date: Nov. 6, 2025

(54) DEVELOPMENT AND VALIDATION OF AN IN VITRO METHOD FOR THE PROGNOSIS OF PATIENTS SUFFERING FROM HER2-POSITIVE BREAST CANCER

(71) Applicants: REVEAL GENOMICS S.L, Barcelona (ES); FUNDACIÓ DE RECERCA CLÍNIC BARCELONA-INSTITUTD'INVESTIGACIONS BIOMÈDIQUES AUGUST PI I SUNYER, Barcelona (ES); UNIVERSITAT DE BARCELONA, Barcelona (ES); HOSPITAL CLINIC DE BARCELONA, Barcelona (ES); UNIVERSITÀ DEGLI STUDI DI PADOVA, Padova (IT)

(72) Inventors: Aleix PRAT APARICIO, Barcelona (ES); Fara BRASÓ MARISTANY, Barcelona (ES); Pierfranco CONTE, Padova (IT); Maria Vittoria DIECI, Padova (IT); Valentina GUARNERI, Padova (IT); patricia VILLAGRASA GONZALEZ, Barcelona (ES)

(73) Assignees: REVEAL GENOMICS S.L, Barcelona (ES); FUNDACIÓ DE RECERCA CLÍNIC BARCELONA-INSTITUTD'INVESTIGACIONS BIOMÈDIQUES AUGUST PI I SUNYER, Barcelona (ES); UNIVERSITAT DE BARCELONA, Barcelona (ES); HOSPITAL CLINIC DE BARCELONA, Barcelona (ES); UNIVERSITÀ DEGLI STUDI DI PADOVA, via VIII febbraio 2 (IT)

(21) Appl. No.: 18/721,628

(22) PCT Filed: Dec. 16, 2022
(86) PCT No.: PCT/EP2022/086493

§ 371 Date: Jun. 18, 2024

(30) Foreign Application Priority Data
Dec. 20, 2021 (EP) 21383165.4

(51) Int. Cl. C12Q 1/6886; A61K 39/00; A61P 35/00; C07K 16/32

(52) U.S. Cl. CPC C12Q 1/6886; A61P 35/00; C07K 16/32; A61K 2039/505; C12Q 2600/106; C12Q 2600/118; C12Q 2600/158

(57) ABSTRACT

The present invention refers to an in vitro method for the prognosis of patients suffering from HER2+ breast cancer, for the prediction of response to anti-HER2 therapies and/or for predicting survival benefit from anti-HER2 therapies.

### CROSS REFERENCE TO RELATED APPLICATIONS

The present application is a U.S. National Phase application under 35 U.S.C. § 371 of International Patent Application No. PCT/EP2022/086493 filed Dec. 16, 2022, which claims priority of European Patent Application No. 21 383 165.4 filed Dec. 20, 2021. The entire contents of which are hereby incorporated by reference.

### FIELD OF THE INVENTION

The present invention refers to the medical field. Particularly, the present invention refers to an in vitro method for the prognosis of patients suffering from HER2+ breast cancer, for the prediction of response to anti-HER2 therapies and/or for predicting survival benefit from anti-HER2 therapies.

### STATE OF THE ART

HER2-positive breast cancer causes a substantial proportion of deaths. In the early stages, (neo)adjuvant chemotherapy and trastuzumab (plus endocrine therapy in hormone receptor-positive disease) have consistently shown significant increases in survival. However, substantial clinical and biological heterogeneity exists in HER2-positive disease, which affects patients' prognosis and treatment benefit.

Strategies to either escalate or de-escalate systemic therapy in early-stage HER2-positive breast cancer to improve survival outcomes and quality of life have been explored, such as decreasing the number of cycles of chemotherapy and the duration of trastuzumab, increasing HER2 blockade with pertuzumab or neratinib, or switching anti-HER2 therapy to trastuzumab emtansine in patients who do not achieve a pathological complete response (pCR) following neoadjuvant therapy. Despite these advances, most patients with early-stage, HER2-positive breast cancer are cured with chemotherapy and trastuzumab alone.

Several variables beyond tumor burden have been associated with patients' prognosis and/or treatment response in early-stage, HER2-positive breast cancer. For example, percentage of stromal tumor-infiltrating lymphocytes (TILs), hormone receptor status, and the intrinsic molecular subtypes of breast cancer are all linked to response and/or survival. However, decisions today about escalation or de-escalation of systemic therapies are based on tumor size, nodal status, expression of the hormone receptors, and response to neoadjuvant therapy (i.e., pCR or not). Therefore, a tool that integrates these multiple variables together to help guide therapy in early-stage, HER2-positive breast cancer is needed and would perform better than any single feature.

Although in 2020 we reported HER2DX to build a multivariable prognostic score in early-stage HER2-positive breast cancer, which integrates information including tumor size and nodal staging, TILs, intrinsic molecular subtype, and the expression of 13 individual genes, the present invention aims to validate new signatures which can be used to improve the prognosis of patients suffering from HER2+ breast cancer, the prediction of response to anti-HER2 therapies and/or the prediction survival benefit from anti-HER2 therapies.

### DESCRIPTION OF THE INVENTION

### Brief Description of the Invention

As explained above, the present invention refers to an in vitro method for the prognosis of patients suffering from HER2+ breast cancer, for the prediction of response to anti-HER2 therapies and/or for predicting survival benefit from anti-HER2 therapies. Particularly, the inventors of the present invention have developed an improved assay, called HER2DX assay, wherein the gene expression of up to 27 genes [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 and/or TCAP], optionally in combination with clinical features, is used for the prognosis of patients suffering from HER2+ breast cancer or for the prediction of response to anti-HER2 therapies. This means that any of the above identified 27 genes can be used in the context of the present invention, preferably any combination thereof comprising between 2 and 27 genes, for the prognosis of patients suffering from HER2+ breast cancer, for the prediction of response to anti-HER2 therapies and/or for predicting survival benefit from anti-HER2 therapies.

On the other hand, the gene expression of up to 4 genes [CD86, FGFR2, ERBB3 and/or FA2H] is used for predicting survival benefit from anti-HER2 therapies. This means that any of the above identified 4 genes can be used in the context of the present invention, preferably any combination thereof comprising between 2 and 4 genes, for the prediction of response to anti-HER2 therapies and/or for predicting survival benefit from anti-HER2 therapies.

In a preferred embodiment, the 27 gene variables included in HER2DX supervised learning algorithm are split into 4 gene expression signatures tracking immune infiltration, tumor cell proliferation, luminal differentiation, and the expression of the HER2 amplicon, giving rise to a single score. The 4 gene expression signatures are as follows:

HER2DX Risk Score (for the Prognosis of Patients Suffering from HER2+ Breast Cancer):
Immune signature (IGG) (14 genes): [CD27, CD79A, HLA-C, IGJ, IGKC, IGL, IGLV3-25, IL2RG, CXCL8, LAX1, NTN3, PIM2, POU2AF1 and/or TNFRSF17].
Tumor cell proliferation signature (PROLIF) (4 genes): [EXO1, ASPM, NEK2 and/or KIF23].
Luminal differentiation signature (LUM) (5 genes): [BCL2, DNAJC12, AGR3, AFF3 and/or ESR1].
HER2 amplicon signature (HER2) (4 genes): [ERBB2, GRB7, STARD3 and/or TCAP].

The coefficients of the HER2DX prognostic risk score full model are as follows: LUM: −0.087, PROLIF: 0.129, HER2: 0.00, IGG: −0.328, T_Stage (T1 vs T2-4): 0 vs. 0.431, N_Stage (NO vs N1): 0 vs. 1.151, N_Stage (NO vs. N2-3): 0 vs. 1.58.

### HER2DX pCR Probability Score (for the Prediction of Response to Anti-HER2 Therapies):

Immune signature (IGG) (14 genes): [CD27, CD79A, HLA-C, IGJ, IGKC, IGL, IGLV3-25, IL2RG, CXCL8, LAX1, NTN3, PIM2, POU2AF1 and/or TNFRSF17].
Tumor cell proliferation signature (PROLIF) (4 genes): [EXO1, ASPM, NEK2 and/or KIF23].
Luminal differentiation signature (LUM) (5 genes): [BCL2, DNAJC12, AGR3, AFF3 and/or ESR1].
HER2 amplicon signature (HER2) (4 genes): [ERBB2, GRB7, STARD3 and/or TCAP].

The coefficients of the HER2DX pCR probability score model are as follows: LUM: −0.365. PROLIF: 0.374. HER2: 0.215. IGG: 0.184. T_Stage (T1 vs. T2-4): 0 vs. −0.630. N_Stage (NO vs N1-3): 0 vs. −0.251.

In order to validate these signatures, 434 HER2+ tumors from the Short-HER trial were used to train a prognostic risk model; 268 cases from an independent cohort were used to verify the accuracy of the HER2DX risk score. In addition, 116 cases treated with neoadjuvant anti-HER2-based chemotherapy were used to train a predictive model of pathological complete response (pCR); two independent cohorts of 91 and 67 cases were used to verify the accuracy of the HER2DX pCR probability score.

HER2DX variables were associated with good outcome (i.e., immune, and luminal) and poor outcome (i.e., proliferation, and tumor and nodal staging). In an independent cohort, continuous HER2DX risk score was significantly associated with disease-free survival (DFS) (p=0.002); the 5-year DFS in the low-risk group was 95.3% (92.4-98.2%). For the neoadjuvant pCR predictor training cohort, HER2DX variables were associated with pCR (i.e., immune, proliferation and HER2 amplicon) and non-pCR (i.e., luminal, and tumor and nodal staging). In both independent test set cohorts, continuous HER2DX pCR probability score was significantly associated with pCR (p<0.0001). A weak negative correlation was found between the two HER2DX scores (correlation coefficient −0.19).

The two HER2DX tests provide accurate estimates of the risk of recurrence, and the probability to achieve a pCR, in early-stage HER2-positive breast cancer. Thus, in conclusion, HER2DX is a novel 27-gene expression and clinical feature-based classifier intended for clinical use for patients with early-stage HER2-positive breast cancer. The assay optionally integrates clinical data with genomic data capturing tumor- and immune-related biology and predicts two different clinical endpoints, namely, long-term survival and probability of achieving a pCR. We validate these two novel assays, one for survival and one for predicting pCR, using multiple datasets, thus providing a high level of technical and clinical validation. Interestingly, the HER2DX risk score and HER2DX pCR probability score provide complementary information, opening an opportunity to better guide therapy through use of predictions of both response and survival.

In a preferred embodiment 23 out of the 27 genes [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and/or ESR1] were used for the prognosis of patients suffering from HER2+ breast cancer, and 27 genes [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 and/or TCAP] were used for the prediction of response to anti-HER2 therapies in patients suffering from HER2+ breast cancer, or for classifying patients into responder or non-responder patients to anti-HER2 therapies

So, the first embodiment of the present invention refers to an in vitro method for the prognosis of patients suffering from HER2+ breast cancer, which comprises measuring the level of expression of at least a gene selected from the group comprising: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and/or ESR1], or any combination thereof comprising between 2 and 23 of said genes, in a biological sample obtained from the patient, wherein:
a. A statistically significant overexpression of at least one gene selected from the group comprising: [CD27, CD79A, HLA-C, IGJ, IGKC, IGL, IGLV3-25, IL2RG, CXCL8, LAX1, NTN3, PIM2, POU2AF1 and/or TNFRSF17], or any combination thereof comprising between 2 and 14 of said genes, with respect to a pre-established reference level of expression, is indicative of good prognosis, and/or
b. A statistically significant overexpression of at least one gene selected from the group comprising: [EXO1, ASPM, NEK2 and/or KIF23], or any combination thereof comprising between 2 and 4 of said genes, with respect to a pre-established reference level of expression, is indicative of poor prognosis, and/or
c. A statistically significant overexpression of at least one gene selected from the group comprising: [BCL2, DNAJC12, AGR3, AFF3 and/or ESR1], or any combination thereof comprising between 2 and 5 of said genes, with respect to a pre-established reference level of expression, is indicative of good prognosis.

The second embodiment of the present invention refers to an in vitro method for the prognosis of patients suffering from HER2+ breast cancer, which comprises measuring the level of expression of at least a gene selected from the group comprising: [CD27, CD79A, HLA-C, IGJ, IGKC, IGL, IGLV3-25, IL2RG, CXCL8, LAX1, NTN3, PIM2, POU2AF1 and/or TNFRSF17], or any combination thereof comprising between 2 and 14 of said genes, wherein a statistically significant overexpression of at least one gene selected from the group comprising: [CD27, CD79A, HLA-C, IGJ, IGKC, IGL, IGLV3-25, IL2RG, CXCL8, LAX1, NTN3, PIM2, POU2AF1 and/or TNFRSF17], or any combination thereof comprising between 2 and 14 of said genes, with respect to a pre-established reference level of expression, is indicative of good prognosis.

The third embodiment of the present invention refers to an in vitro method for the prognosis of patients suffering from HER2+ breast cancer, which comprises measuring the level of expression of at least a gene selected from the group comprising: [EXO1, ASPM, NEK2 and/or KIF23], or any combination thereof comprising between 2 and 4 of said genes, wherein a statistically significant overexpression of at least one gene selected from the group comprising: [EXO1, ASPM, NEK2 and/or KIF23], or any combination thereof comprising between 2 and 4 of said genes, with respect to a pre-established reference level of expression, is indicative of poor prognosis.

The fourth embodiment of the present invention refers to an in vitro method for the prognosis of patients suffering from HER2+ breast cancer, which comprises measuring the level of expression of at least a gene selected from the group comprising: [BCL2, DNAJC12, AGR3, AFF3 and/or ESR1], or any combination thereof comprising between 2 and 5 of said genes, wherein a statistically significant overexpression of at least one gene selected from the group comprising: [BCL2, DNAJC12, AGR3, AFF3 and/or ESR1], or any combination thereof comprising between 2 and 5 of said genes, with respect to a pre-established reference level of expression, is indicative of good prognosis.

The fourth embodiment of the present invention refers to an in vitro method for the prediction of response to anti-HER2 therapies in patients suffering from HER2+ breast cancer, or for classifying patients into responder or non-responder patients to anti-HER2 therapies, which comprises measuring the level of expression of at least a gene selected from the group comprising: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 and/or TCAP], or any combination thereof comprising between 2 and 27 of said genes, in a biological sample obtained from the patient, wherein:
a. A statistically significant overexpression of at least one gene selected from the group comprising: [CD27, CD79A, HLA-C, IGJ, IGKC, IGL, IGLV3-25, IL2RG, CXCL8, LAX1, NTN3, PIM2, POU2AF1 and/or TNFRSF17], or any combination thereof comprising between 2 and 14 genes, with respect to a pre-established reference level of expression, is indicative that the patient is a responder patient to anti-HER2 therapies, and/or
b. A statistically significant overexpression of at least one gene selected from the group comprising: [EXO1, ASPM, NEK2 and/or KIF23], or any combination thereof comprising between 2 and 4 genes, with respect to a pre-established reference level of expression, is indicative that the patient is a responder patient to anti-HER2 therapies, and/or
c. A statistically significant overexpression of at least one gene selected from the group comprising: [BCL2, DNAJC12, AGR3, AFF3 and/or ESR1], or any combination thereof comprising between 2 and 5 genes, with respect to a pre-established reference level of expression, is indicative that the patient is a non-responder patient to anti-HER2 therapies, and/or
d. A statistically significant overexpression of at least one gene selected from the group comprising: [ERBB2, GRB7, STARD3 and/or TCAP], or any combination thereof comprising between 2 and 4 genes, with respect to a pre-established reference level of expression, is indicative that the patient is a responder patient to anti-HER2 therapies.

The fifth embodiment of the present invention refers to an in vitro method for the prediction of response to anti-HER2 therapies in patients suffering from HER2+ breast cancer, or for classifying patients into responder or non-responder patients to anti-HER2 therapies, which comprises measuring the level of expression of at least a gene selected from the group comprising: [CD27, CD79A, HLA-C, IGJ, IGKC, IGL, IGLV3-25, IL2RG, CXCL8, LAX1, NTN3, PIM2, POU2AF1 and/or TNFRSF17], or any combination thereof comprising between 2 and 14 of said genes, with respect to a pre-established reference level of expression, in a biological sample obtained from the patient, wherein a statistically significant overexpression of at least one gene selected from the group comprising: [CD27, CD79A, HLA-C, IGJ, IGKC, IGL, IGLV3-25, IL2RG, CXCL8, LAX1, NTN3, PIM2, POU2AF1 and/or TNFRSF17], or any combination thereof comprising between 2 and 14 of said genes, with respect to a pre-established reference level of expression, is indicative that the patient is a responder patient to anti-HER2 therapies.

The sixth embodiment of the present invention refers to an in vitro method for the prediction of response to anti-HER2 therapies in patients suffering from HER2+ breast cancer, or for classifying patients into responder or non-responder patients to anti-HER2 therapies, which comprises measuring the level of expression of at least a gene selected from the group comprising: [EXO1, ASPM, NEK2 and/or KIF23], or any combination thereof comprising between 2 and 4 of said genes, with respect to a pre-established reference level of expression, wherein a statistically significant overexpression of at least one gene selected from the group comprising: [EXO1, ASPM, NEK2 and/or KIF23], or any combination thereof comprising between 2 and 4 of said genes, with respect to a pre-established reference level of expression, is indicative that the patient is a responder patient to anti-HER2 therapies.

The seventh embodiment of the present invention refers to an in vitro method for the prediction of response to anti-HER2 therapies in patients suffering from HER2+ breast cancer, or for classifying patients into responder or non-responder patients to anti-HER2 therapies, which comprises measuring the level of expression of at least a gene selected from the group comprising: [BCL2, DNAJC12, AGR3, AFF3 and/or ESR1], or any combination thereof comprising between 2 and 5 of said genes, with respect to a pre-established reference level of expression, wherein a statistically significant overexpression of at least one gene selected from the group comprising: [BCL2, DNAJC12, AGR3, AFF3 and/or ESR1], or any combination thereof comprising between 2 and 5 of said genes, with respect to a pre-established reference level of expression, is indicative that the patient is a non-responder patient to anti-HER2 therapies.

The eight embodiment of the present invention refers to an in vitro method for the prediction of response to anti-HER2 therapies in patients suffering from HER2+ breast cancer, or for classifying patients into responder or non-responder patients to anti-HER2 therapies, which comprises measuring the level of expression of at least a gene selected from the group comprising: [ERBB2, GRB7, STARD3 and/or TCAP], or any combination thereof comprising between 2 and 4 of said genes, with respect to a pre-established reference level of expression, wherein a statistically significant overexpression of at least one gene selected from the group comprising: [ERBB2, GRB7, STARD3 and/or TCAP], or any combination thereof comprising between 2 and 4 of said genes, with respect to a pre-established reference level of expression, is indicative that the patient is a responder patient to anti-HER2 therapies.

The ninth embodiment of the present invention refers to the in vitro use of at least a gene selected from the group comprising: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and/or ESR1], or any combination thereof comprising between 2 and 23 genes, for the prognosis of patients suffering from HER2+ breast cancer.

The tenth embodiment of the present invention refers to the in vitro use of at least a gene selected from the group comprising: [CD27, CD79A, HLA-C, IGJ, IGKC, IGL, IGLV3-25, IL2RG, CXCL8, LAX1, NTN3, PIM2, POU2AF1 and/or TNFRSF17], or any combination thereof comprising between 2 and 14 genes, for the prognosis of patients suffering from HER2+ breast cancer.

The eleventh embodiment of the present invention refers to the in vitro use of at least a gene selected from the group comprising: [EXO1, ASPM, NEK2 and/or KIF23], or any combination thereof comprising between 2 and 4 genes, for the prognosis of patients suffering from HER2+ breast cancer.

The twelfth embodiment of the present invention refers to the in vitro use of at least one gene selected from the group comprising: [BCL2, DNAJC12, AGR3, AFF3 and/or ESR1], or any combination thereof comprising between 2 and 5 genes, for the prognosis of patients suffering from HER2+ breast cancer.

The thirteenth embodiment of the present invention refers to the in vitro use of at least a gene selected from the group comprising: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and/or ESR1, ERBB2, GRB7, STARD3 and/or TCAP], or any combination thereof comprising between 2 and 27 genes, for the prediction of response to anti-HER2 therapies in patients suffering from HER2+ breast cancer, or for classifying patients into responder or non-responder patients to anti-HER2 therapies.

The fourteenth embodiment of the present invention refers to the in vitro use of at least a gene selected from the group comprising: [CD27, CD79A, HLA-C, IGJ, IGKC, IGL, IGLV3-25, IL2RG, CXCL8, LAX1, NTN3, PIM2, POU2AF1 and/or TNFRSF17], or any combination thereof comprising between 2 and 14 genes, for the prediction of response to anti-HER2 therapies in patients suffering from HER2+ breast cancer, or for classifying patients into responder or non-responder patients to anti-HER2 therapies.

The fifteenth embodiment of the present invention refers to the in vitro use of at least a gene selected from the group comprising: [EXO1, ASPM, NEK2 and/or KIF23], or any combination thereof comprising between 2 and 4 genes, for the prediction of response to anti-HER2 therapies in patients suffering from HER2+ breast cancer, or for classifying patients into responder or non-responder patients to anti-HER2 therapies.

The sixteenth embodiment of the present invention refers to the in vitro use of at least a gene selected from the group comprising: [BCL2, DNAJC12, AGR3, AFF3 and/or ESR1], or any combination thereof comprising between 2 and 5 genes, for the prediction of response to anti-HER2 therapies in patients suffering from HER2+ breast cancer, or for classifying patients into responder or non-responder patients to anti-HER2 therapies.

The seventeenth embodiment of the present invention refers to the in vitro use of at least a gene selected from the group comprising: [ERBB2, GRB7, STARD3 and/or TCAP], or any combination thereof comprising between 2 and 4 genes, for the prediction of response to anti-HER2 therapies in patients suffering from HER2+ breast cancer, or for classifying patients into responder or non-responder patients to anti-HER2 therapies.

In a preferred embodiment, the present invention further comprises identifying the nodal status (pN1) and/or tumor staging (pT2-4) wherein the identification of nodal status N1-3 and/or tumor status T2-4 is indicative of bad prognosis or that the patient is a non-responder patient to anti-HER2 therapies.

In a preferred embodiment, the patient is suffering from HER2+ breast cancer.

In a preferred embodiment, the sample is selected form: tissue, blood, serum or plasma.

In a preferred embodiment, the anti-HER2 therapy is a drug selected from: trastuzumab, pertuzumab, lapatinib, pyrotinib, poziotinib, tucatinib, neratinib, trastuzumab deruxtecan, SYD985 or ado-trastuzumab emtansine.

The eighteenth embodiment of the present invention refers to a kit comprising reagents for measuring the level of expression of a group of genes consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and/or ESR1], or any combination thereof comprising between 2 and 23 genes, preferably consisting of: [CD27, CD79A, HLA-C, IGJ, IGKC, IGL, IGLV3-25, IL2RG, CXCL8, LAX1, NTN3, PIM2, POU2AF1 and/or TNFRSF17], [EXO1, ASPM, NEK2 and/or KIF23], or [BCL2, DNAJC12, AGR3, AFF3 and/or ESR1].

The nineteenth embodiment of the present invention refers to a kit comprising reagents for measuring the level of expression of a group of genes consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and/or ESR1, ERBB2, GRB7, STARD3 and/or TCAP], or any combination thereof comprising between 2 and 27 genes, preferably consisting of: [CD27, CD79A, HLA-C, IGJ, IGKC, IGL, IGLV3-25, IL2RG, CXCL8, LAX1, NTN3, PIM2, POU2AF1 and/or TNFRSF17], or [EXO1, ASPM, NEK2 and/or KIF23], or [BCL2, DNAJC12, AGR3, AFF3 and/or ESR1], or [ERBB2, GRB7, STARD3 and/or TCAP].

The twentieth embodiment of the present invention refers to anti-HER2 therapy, or any pharmaceutical composition comprising thereof, optionally including pharmaceutically acceptable excipients or carriers, for use in the treatment of patients suffering from HER2+ breast cancer wherein the patient has been classified as responder patient because it is characterized by showing a statistically higher expression level, as compared with a pre-established threshold value, of at least a gene selected from the group comprising: [CD27, CD79A, HLA-C, IGJ, IGKC, IGL, IGLV3-25, IL2RG, CXCL8, LAX1, NTN3, PIM2, POU2AF1 and/or TNFRSF17], or [EXO1, ASPM, NEK2 and/or KIF23] or [ERBB2, GRB7, STARD3 and/or TCAP], wherein the anti-HER2 therapy is optionally selected from: trastuzumab, pertuzumab, lapatinib, pyrotinib, poziotinib, tucatinib, neratinib, trastuzumab deruxtecan, SYD985 or ado-trastuzumab emtansine. In this sense, the present invention also refers to a method for treating a patient suffering from HER2+ breast cancer which comprised the administration of a therapeutically effective dose or amount of anti-HER2 compound, once the patient has been previously classified as responder patient following any of the above-cited methods.

The twenty-first embodiment of the present invention refers to an in vitro method for predicting survival benefit from anti-HER2 therapy of patients suffering from HER2+ breast cancer treated with anti-HER2 therapies which comprises measuring the level of expression of at least a gene selected from the group comprising: [CD86, FGFR2, ERBB3 and/or FA2H] in a biological sample obtained from the patient, wherein a statistically significant overexpression of at least one gene selected from the group comprising: [CD86, FGFR2, ERBB3 and/or FA2H], or any combination thereof comprising between 2 and 4 genes, with respect to a pre-established reference level of expression, is indicative of survival benefit of patients suffering from HER2+ breast cancer treated with anti-HER2 therapies.

The twenty-second embodiment of the present invention refers to the in vitro use of at least a gene selected from the group comprising: [CD86, FGFR2, ERBB3 and/or FA2H] for predicting survival benefit of patients suffering from HER2+ breast cancer treated with anti-HER2 therapies.

The twenty-third embodiment of the present invention refers to a kit comprising reagents for measuring the level of expression of a group of genes consisting of [CD86, FGFR2, ERBB3 and/or FA2H].

Particularly, although the method of the invention involves up to 23 or 27 genes, it is important to consider that the present invention offers strong data showing that the combination of at least 2 genes, tracking the luminal, proliferation and immune pathways is prognostic in early-stage HER2+ breast cancer (Example 2.6) and that the combination of at least 2 genes tracking the luminal, HER2 amplicon, proliferation and immune signatures is predictive of pathological complete response (pCR) (Example 2.7). So, in a preferred embodiment, the present invention also refers to:

In vitro method for identifying biomarker signatures for the prognosis of patients suffering from HER2+ breast cancer, which comprises: a) Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1], in a biological sample obtained from the patient; b) determining a combination score value by calculating the ratio of the expression of the 2 genes; and c) wherein if a deviation of the combination score value is identified, as compared with a pre-established reference value, this is indicative that the biomarker signature may be used for the prognosis of patients suffering from HER2+ breast cancer.

In vitro method for the prognosis of patients suffering from HER2+ breast cancer which comprises: a) Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1], in a biological sample obtained from the patient; b) determining a combination score value by calculating the ratio of the expression of the 2 genes; and c) wherein if a deviation of the combination score value is identified, as compared with a pre-established reference value, this is indicative of the prognosis of patients suffering from HER2+ breast cancer.

In vitro method for the prognosis of patients suffering from HER2+ breast cancer which comprises: a) Measuring the level of expression of at least two genes selected from the group consisting of [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1], in a biological sample obtained from the patient; b) determining a combination score value by calculating the ratio of the expression of the 2 genes, wherein the ratio is calculated by:
i. Combining a first gene comprised in the immune signature with a second gene comprised in the tumor cell proliferation signature; or
ii. Combining a first gene comprised in the immune signature with a second gene comprised in the luminal differentiation signature; or
iii. Combining a first gene comprised in the luminal differentiation signature with a second gene comprised in the tumor cell proliferation signature; or
iv. Combining a first gene comprised in the immune signature selected from the group consisting of CD79A, CD27, IGJ, POU2AF1, TNFRSF17, IL2RG, PIM2 or IGL with a second gene comprised in the immune signature selected from the group consisting of: CD27, CXCL8, HLA-C, IGLV3-25, IL2RG, LAX1, NTN3, PIM2 or POU2AF1;
   c) wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EXO1, ASPM, NEK2 or KIF23] and the luminal differentiation signature comprises the genes: [BCL2, DNAJC12, AGR3, AFF3 or ESR1]; and
   d) wherein if a deviation of the combination score value is identified, as compared with a pre-established reference value, is indicative of good prognosis.

In vitro method for the prognosis of patients suffering from HER2+ breast cancer which comprises: a) Measuring the level of expression of at least two genes selected from the gene combinations of Table 7A, in a biological sample obtained from the patient; b) determining a combination score value by calculating the ratio of the expression of the 2 genes; and c) herein if a deviation of the combination score value is identified, as compared with a pre-established reference value, is indicative of good prognosis.

In vitro method for the prognosis of patients suffering from HER2+ breast cancer which comprises: a) Measuring the level of expression of at least two genes selected from the group consisting of [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1], in a biological sample obtained from the patient; b) determining a combination score value by calculating the ratio of the expression of the 2 genes, wherein the ratio is calculated by:
i. Combining a first gene comprised in the tumor cell proliferation signature with a second gene comprised in the immune signature; or
ii. Combining a first gene comprised in the luminal differentiation signature with a second gene comprised in the immune signature; or
iii. Combining a first gene comprised in the tumor cell proliferation signature with a second gene comprised in the luminal differentiation signature; or
iv. Combining a first gene comprised in the immune signature selected from the group consisting of CD27, CXCL8, HLA-C, IGLV3-25, IL2RG, LAX1, NTN3, PIM2 or POU2AF1 with a second gene comprised in the immune signature selected from the group consisting of: CD79A, CD27, IGJ, POU2AF1, TNFRSF17, IL2RG, PIM2 or IGL;
   c) wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EXO1, ASPM, NEK2 or KIF23] and the luminal differentiation signature comprises the genes: [BCL2, DNAJC12, AGR3, AFF3 or ESR1]; and
   d) wherein if a deviation of the combination score value is identified, as compared with a pre-established reference value, is indicative of poor prognosis.

In vitro method for the prognosis of patients suffering from HER2+ breast cancer which comprises: a) Measuring the level of expression of at least two genes selected from the gene combinations of Table 7B, in a biological sample obtained from the patient; b) determining a combination score value by calculating the ratio of the expression of the 2 genes; and c) wherein if a deviation of the combination score value is identified, as compared with a pre-established reference value, is indicative of poor prognosis.

In vitro method for the prognosis of patients suffering from HER2+ breast cancer which comprises measuring the level of expression of a group of genes consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and ESR1].

In vitro method for identifying biomarker signatures for the prediction of response to anti-HER2 therapies in patients suffering from HER2+ breast cancer, or for classifying patients into responder or non-responder patients to anti-HER2 therapies, which comprises: a) Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP], in a biological sample obtained from the patient; b) determining a combination score value by calculating the ratio of the expression of the 2 genes; and c) wherein if a deviation of the combination score value is identified, as compared with a pre-established reference value, this is indicative that the biomarker signature may be used for the prediction of response to anti-HER2 therapies in patients suffering from HER2+ breast cancer, or for classifying patients into responder or non-responder patients to anti-HER2 therapies.

In vitro method for the prediction of response to anti-HER2 therapies in patients suffering from HER2+ breast cancer, or for classifying patients into responder or non-responder patients to anti-HER2 therapies which comprises: a) Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP], in a biological sample obtained from the patient; b) determining a combination score value by calculating the ratio of the expression of the 2 genes; and c) wherein if a deviation of the combination score value is identified, as compared with a pre-established reference value, this is indicative of the response to anti-HER2 therapies in patients suffering from HER2+ breast cancer.

In vitro method for the prediction of response to anti-HER2 therapies in patients suffering from HER2+ breast cancer, or for classifying patients into responder or non-responder patients to anti-HER2 therapies which comprises: a) Measuring the level of expression of at least two genes selected from the group consisting of [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP], in a biological sample obtained from the patient; d) determining a combination score value by calculating the ratio of the expression of the 2 genes, wherein the ratio is calculated by:
i. Combining a first gene comprised in the immune signature with a second gene comprised in the luminal differentiation signature; or
ii. Combining a first gene comprised in the tumor cell proliferation signature with a second gene comprised in the luminal differentiation signature; or
iii. Combining a first gene comprised in the HER2 amplicon signature with a second gene comprised in the immune signature; or
iv. Combining a first gene comprised in the HER2 amplicon signature with a second gene comprised in the tumor cell proliferation signature; or
v. Combining a first gene comprised in the HER2 amplicon signature with a second gene comprised in the luminal differentiation signature; or
vi. Combining a first gene comprised in the immune signature selected from the group consisting of: IGKC, IGL or LAX1 with a second gene comprised in the immune signature selected from the group consisting of: HLA-C, CD27, IGJ, LAX1, NTN3, PIM2, POU2AF1 or TNFRSF17; or
vii. Combining a first gene comprised in the luminal differentiation signature selected from the group consisting of: AFF3, BCL2 or DNAJC12, with a second gene comprised in the luminal differentiation signature selected from the group consisting of: ESR1 or AGR3; or
viii. Combining the first gene ASPM comprised in the tumor cell proliferation signature with the second gene NEK2 comprised in the tumor cell proliferation signature; and
   c) wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EXO1, ASPM, NEK2 or KIF23], the luminal differentiation signature comprises the genes: [BCL2, DNAJC12, AGR3, AFF3 or ESR1] and the HER2 amplicon signature comprises the genes: [ERBB2, GRB7, STARD3 or TCAP], and d) wherein if a deviation of the combination score value is identified, as compared with a pre-established reference value, is an indication that the patients suffering from HER2+ breast cancer may respond to anti-HER2 therapies.

In vitro method for the prediction of response to anti-HER2 therapies in patients suffering from HER2+ breast cancer, or for classifying patients into responder or non-responder patients to anti-HER2 therapies which comprises: a) Measuring the level of expression of at least two genes selected from the gene combinations of Table 9A, in a biological sample obtained from the patient; b) determining a combination score value by calculating the ratio of the expression of the 2 genes; and c) wherein if a deviation of the combination score value is identified, as compared with a pre-established reference value, is an indication that the patients suffering from HER2+ breast cancer may respond to anti-HER2 therapies.

In vitro method for the prediction of response to anti-HER2 therapies in patients suffering from HER2+ breast cancer, or for classifying patients into responder or non-responder patients to anti-HER2 therapies which comprises: a) Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP], in a biological sample obtained from the patient; b) determining a combination score value by calculating the ratio of the expression of the 2 genes, wherein the ratio is calculated by:
i. Combining a first gene comprised in the luminal differentiation signature with a second gene comprised in the immune signature; or
ii. Combining a first gene comprised in the luminal differentiation signature with a second gene comprised in the tumor cell proliferation signature; or
iii. Combining a first gene comprised in the immune differentiation signature with a second gene comprised in the HER2 amplicon signature; or
iv. Combining a first gene comprised in the tumor cell proliferation signature with a second gene comprised in the HER2 amplicon signature; or
v. Combining a first gene comprised in the luminal differentiation signature with a second gene comprised in the HER2 amplicon signature; or
vi. Combining a first gene comprised in the immune signature selected from the group consisting of: HLA-C, CD27, IGJ, LAX1, NTN3, PIM2, POU2AF1 or TNFRSF17 with a second gene comprised in the immune signature selected from the group consisting of: IGKC, IGL or LAX1; or
vii. Combining a first gene comprised in the luminal differentiation signature selected from the group consisting of: ESR1 or AGR3 with a second gene comprised in the luminal differentiation signature selected from the group consisting of: AFF3, BCL2, or DNAJC12; or
viii. Combining the first gene NEK2 comprised in the tumor cell proliferation signature with the second gene ASPM comprised in the tumor cell proliferation signature; and
   c) wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EXO1, ASPM, NEK2 or KIF23], the luminal differentiation signature comprises the genes: [BCL2, DNAJC12, AGR3, AFF3 or ESR1] and the HER2 amplicon signature comprises the genes: [ERBB2, GRB7, STARD3 or TCAP], and d) wherein if a deviation of the combination score value is identified, as compared with a pre-established reference value, is an indication that the patients suffering from HER2+ breast cancer may not respond to anti-HER2 therapies.

In vitro method for the prediction of response to anti-HER2 therapies in patients suffering from HER2+ breast cancer, or for classifying patients into responder or non-responder patients to anti-HER2 therapies which comprises: a) Measuring the level of expression of at least two genes selected from the gene combinations of Table 9B, in a biological sample obtained from the patient; b) determining a combination score value by calculating the ratio of the expression of the 2 genes; and c) wherein if a deviation of the combination score value is identified, as compared with a pre-established reference value, is an indication that the patients suffering from HER2+ breast cancer may not respond to anti-HER2 therapies.

In vitro method for the prediction of response to anti-HER2 therapies in patients suffering from HER2+ breast cancer, or for classifying patients into responder or non-responder patients to anti-HER2 therapies which comprises measuring the level of expression of a group of genes consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 and TCAP].

In a preferred embodiment the method further comprises identifying the nodal status (pN1) and/or tumor staging (pT2-4) wherein the identification of nodal status N1-3 and/or tumor status T2-4 is indicative of bad prognosis or that the patient is a non-responder patient to anti-HER2 therapies.

In a preferred embodiment the patient is suffering from HER2+ breast cancer.

In a preferred embodiment the sample is selected form: tissue, blood, serum or plasma.

In a preferred embodiment the anti-HER2 therapy is a drug selected from: trastuzumab, pertuzumab, lapatinib, pyrotinib, poziotinib, tucatinib, neratinib, trastuzumab deruxtecan, SYD985 or ado-trastuzumab emtansine.

In vitro use at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1] for identifying biomarker signatures for the prognosis of patients suffering from HER2+ breast cancer.

In vitro use of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1] for the prognosis of patients suffering from HER2+ breast cancer.

In vitro use of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1] wherein the first gene is comprised in the immune signature and the second gene is comprised in the tumor cell proliferation signature, or wherein the first gene is comprised in the immune signature and the second gene is comprised in the luminal differentiation signature, or wherein the first gene is comprised in the luminal differentiation signature and the second gene is comprised in the tumor cell proliferation signature, or wherein the first gene is comprised in the immune signature and is selected from the group consisting of: CD79A, CD27, IGJ, POU2AF1, TNFRSF17, IL2RG, PIM2 or IGL and the second gene is comprised in the immune signature and is selected from the group consisting of: CD27, CXCL8, HLA-C, IGLV3-25, IL2RG, LAX1, NTN3, PIM2 or POU2AF1; and wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EXO1, ASPM, NEK2 or KIF23], and the luminal differentiation signature comprises the genes: [BCL2, DNAJC12, AGR3, AFF3 or ESR1]; for the prognosis of patients suffering from HER2+ breast cancer.

In vitro use of at least two genes selected from the gene combinations of Table 7A for the prognosis of patients suffering from HER2+ breast cancer.

In vitro use of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1] wherein the first gene is comprised in the tumor cell proliferation signature and the second gene is comprised in the immune signature, or wherein the first gene is comprised in the luminal differentiation signature and the second gene is comprised in the immune signature, or wherein the first gene is comprised in the tumor cell proliferation signature and the second gene is comprised in the luminal differentiation signature, or wherein the first gene is comprised in the immune signature and it is selected from the group consisting of CD27, CXCL8, HLA-C, IGLV3-25, IL2RG, LAX1, NTN3, PIM2 or POU2AF1 and the second gene is comprised in the immune signature and it is selected from the group consisting of: CD79A, CD27, IGJ, POU2AF1, TNFRSF17, IL2RG, PIM2 or IGL; and wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EXO1, ASPM, NEK2 or KIF23], and the luminal differentiation signature comprises the genes: [BCL2, DNAJC12, AGR3, AFF3 or ESR1]; for the prognosis of patients suffering from HER2+ breast cancer.

In vitro use of at least two genes selected from the gene combinations of Table 7B for the prognosis of patients suffering from HER2+ breast cancer.

In vitro use of a group of genes consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and ESR1] for the prognosis of patients suffering from HER2+ breast cancer.

In vitro use of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP] for identifying biomarker signatures for the prediction of response to anti-HER2 therapies in patients suffering from HER2+ breast cancer, or for classifying patients into responder or non-responder patients to anti-HER2 therapies.

In vitro use of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP] for the prediction of response to anti-HER2 therapies in patients suffering from HER2+ breast cancer, or for classifying patients into responder or non-responder patients to anti-HER2 therapies.

In vitro use of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP] wherein the first gene is comprised in the immune signature and the second gene is comprised in the luminal differentiation signature; or wherein the first gene is comprised in the tumor cell proliferation signature and the second gene is comprised in the luminal differentiation signature; or wherein the first gene is comprised in the HER2 amplicon signature and the second gene is comprised in the immune signature; or wherein the first gene is comprised in the HER2 amplicon signature and the second gene is comprised in the tumor cell proliferation signature; or wherein the first gene is comprised in the HER2 amplicon signature and the second gene is comprised in the luminal differentiation signature; or wherein the first gene is comprised in the immune signature and it is selected from the group consisting of: IGKC, IGL or LAX1 and the second gene is comprised in the immune signature and it is selected from the group consisting of: HLA-C, CD27, IGJ, LAX1, NTN3, PIM2, POU2AF1 or TNFRSF17; or wherein the first gene is comprised in the luminal differentiation signature and it is selected from the group consisting of: AFF3, BCL2 or DNAJC12 and the second gene is comprised in the luminal differentiation signature and it is selected from the group consisting of: ESR1 or AGR3; or wherein the first gene is ASPM comprised in the tumor cell proliferation and the second gene is NEK2 comprised in the tumor cell proliferation signature; and wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EXO1, ASPM, NEK2 or KIF23], the luminal differentiation signature comprises the genes: [BCL2, DNAJC12, AGR3, AFF3 or ESR1] and the HER2 amplicon signature comprises the genes: [ERBB2, GRB7, STARD3 or TCAP], for the prediction of response to anti-HER2 therapies in patients suffering from HER2+ breast cancer, or for classifying patients into responder or non-responder patients to anti-HER2 therapies.

In vitro use of at least two genes selected from the gene combinations of Table 9A for the prediction of response to anti-HER2 therapies in patients suffering from HER2+ breast cancer, or for classifying patients into responder or non-responder patients to anti-HER2 therapies.

In vitro use of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP] wherein the first gene is comprised in the luminal differentiation signature and the second gene is comprised in immune signature, or wherein the first gene is comprised in the luminal differentiation signature and the second gene is comprised in the tumor cell proliferation signature, or wherein the first gene is comprised in the immune signature and the second gene is comprised in the HER2 amplicon signature, or wherein the first gene is comprised in the tumor cell proliferation signature and the second gene is comprised in the HER2 amplicon signature, or wherein the first gene is comprised in the luminal differentiation signature and the second gene is comprised in the HER2 amplicon signature; or wherein the first gene is comprised in the immune signature and it is selected from the group consisting of: HLA-C, CD27, IGJ, LAX1, NTN3, PIM2, POU2AF1 or TNFRSF17 and the second gene is comprised in the immune signature and it is selected from the group consisting of: IGKC, IGL or LAX1; or wherein the first gene is comprised in the luminal differentiation signature and it is selected from the group consisting of ESR1 or AGR3 and the second gene is comprised in the luminal differentiation signature and it is selected from the group consisting of: AFF3, BCL2, or DNAJC12; or wherein the first gene is NEK2 comprised in the tumor cell proliferation and the second gene is ASPM comprised in the tumor cell proliferation signature; and wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EXO1, ASPM, NEK2 or KIF23], the luminal differentiation signature comprises the genes: [BCL2, DNAJC12, AGR3, AFF3 or ESR1] and the HER2 amplicon signature comprises the genes: [ERBB2, GRB7, STARD3 or TCAP], for the prediction of response to anti-HER2 therapies in patients suffering from HER2+ breast cancer, or for classifying patients into responder or non-responder patients to anti-HER2 therapies.

In vitro use of at least two genes selected from the gene combinations of Table 9B for the prediction of response to anti-HER2 therapies in patients suffering from HER2+ breast cancer, or for classifying patients into responder or non-responder patients to anti-HER2 therapies.

In vitro use of a group of genes consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 and TCAP] for the prediction of response to anti-HER2 therapies in patients suffering from HER2+ breast cancer, or for classifying patients into responder or non-responder patients to anti-HER2 therapies.

Anti-HER2 therapy, or any pharmaceutical composition comprising thereof, optionally including pharmaceutically acceptable excipients or carriers, for use in the treatment of patients suffering from HER2+ breast cancer, wherein the method comprises predicting the response to anti-HER2 therapies in the patients suffering from HER2+ breast cancer or classifying patients into responder or non-responder patients to anti-HER2 therapies, by following the method of the invention.

Anti-HER2 therapy, or any pharmaceutical composition comprising thereof, optionally including pharmaceutically acceptable excipients or carriers, for use in the treatment of patients suffering from HER2+ breast cancer wherein the anti-HER2 therapy is optionally selected from: trastuzumab, pertuzumab, lapatinib, pyrotinib, poziotinib, tucatinib, neratinib, trastuzumab deruxtecan, SYD985 or ado-trastuzumab emtansine.

The present invention also refers to a method for detecting a biomarker signature in a test sample from patients suffering from HER2+ breast cancer the method comprising: a) Contacting the test sample with a reagent specific to the biomarker, b) amplifying the biomarker to produce an amplification product in the test sample; and c) measuring the level by determining the level of the amplification product in the test sample.

In a preferred embodiment, the present invention is a computer-implemented invention, wherein a processing unit (hardware) and a software are configured to: a) Receive the expression level values of any of the above cited biomarkers or signatures, b) process the expression level values received for finding substantial variations or deviations, and c) provide an output through a terminal display of the variation or deviation of the expression level.

In a preferred embodiment, the method of the invention further comprises determining or measuring tumor stage and/or nodal status, for instance by CT scan, ultrasound and/or mammography.

For the purpose of the present invention the following terms are defined:
The term “pre-established reference value”, when referring to the level of the biomarkers described in the present invention, refers to the geometric mean level of the 5 house-keeping genes observed in the patients, namely: GAPD, PUM1, ACTB, RPLP0 and PSMC4. A “reference” value can be a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value must be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data.
The term “variation or deviation” refers to a value which is above or below the pre-established reference value.
By the term “comprising” is meant the inclusion, without limitation, of whatever follows the word “comprising”. Thus, use of the term “comprising” indicates that the listed elements are required or mandatory, but that other elements are optional and may or may not be present.
By “consisting of” is meant the inclusion, with limitation to whatever follows the phrase “consisting of”. Thus, the phrase “consisting of” indicates that the listed elements are required or mandatory, and that no other elements may be present.
“Pharmaceutically acceptable excipient or carrier” refers to an excipient that may optionally be included in the compositions of the invention and that causes no significant adverse toxicological effects to the patient.
By “therapeutically effective dose or amount” of a composition is intended an amount that, when administered as described herein, brings about a positive therapeutic response in a subject having HER2+ breast cancer. The exact amount required will vary from subject to subject, depending on the age, and general condition of the subject, the severity of the condition being treated, mode of administration, and the like. An appropriate “effective” amount in any individual case may be determined by one of ordinary skill in the art using routine experimentation, based upon the information provided herein.

### BRIEF DESCRIPTION OF THE FIGURES

FIG. 1. Summary of the different cohorts of patients evaluated during HER2DX development and validation.

FIG. 2. Survival outcomes of HER2DX low- and high-risk groups in early-stage HER2-positive breast cancer. (A) DRFS in Short-HER dataset; (B) DFS in Short-HER dataset; (C) OS in Short-HER dataset; (D) DFS in an independent combined validation dataset.

FIG. 3. Summary of the variables included in the HER2DX assay and their association with each clinical endpoint.

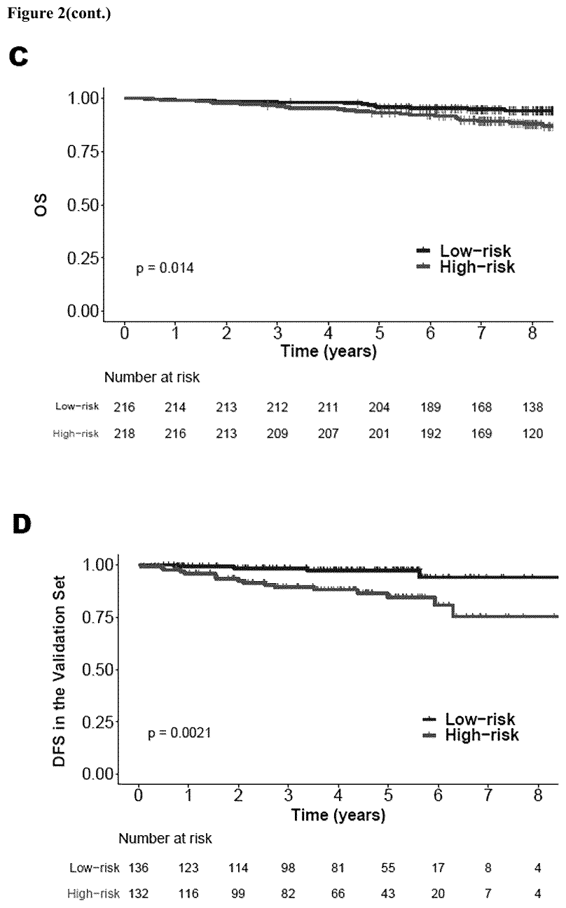

FIG. 4. Survival curves based on CD86 expression and treatment arm. Low and high CD86 expression is defined by the median. Time is defined by months. DMFS96, distant metastasis-free survival at 96 months.

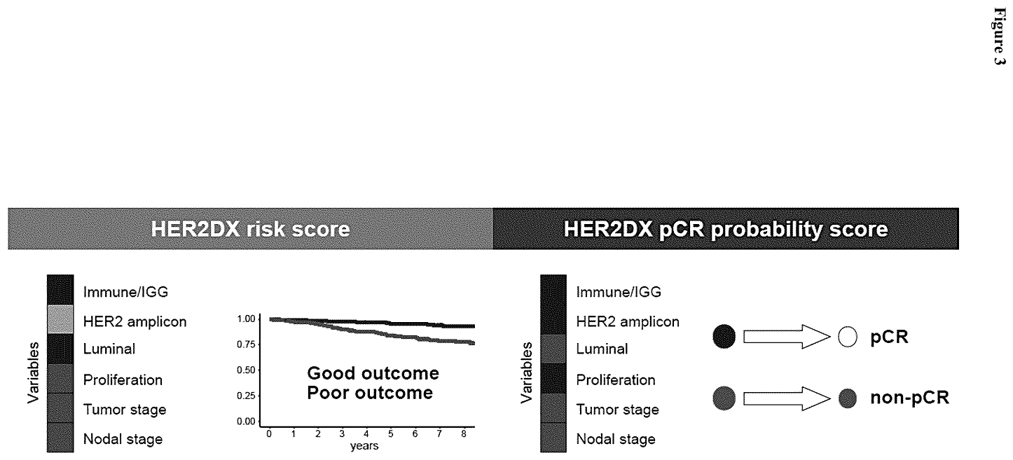

FIG. 5. Venn diagram representing the number of combination scores (2-gene combination scores) significantly associated with survival outcome across the 5 datasets.

FIG. 6. Venn diagram representing the number of combination scores (2-gene combination scores) significantly associated with pCR in the 3 datasets.

### DETAILED DESCRIPTION OF THE INVENTION

The present invention is illustrated by means of the examples set below, without the intention of limiting its scope of protection.

### Example 1. Material and Methods

### Example 1.1. Study Design and Participants

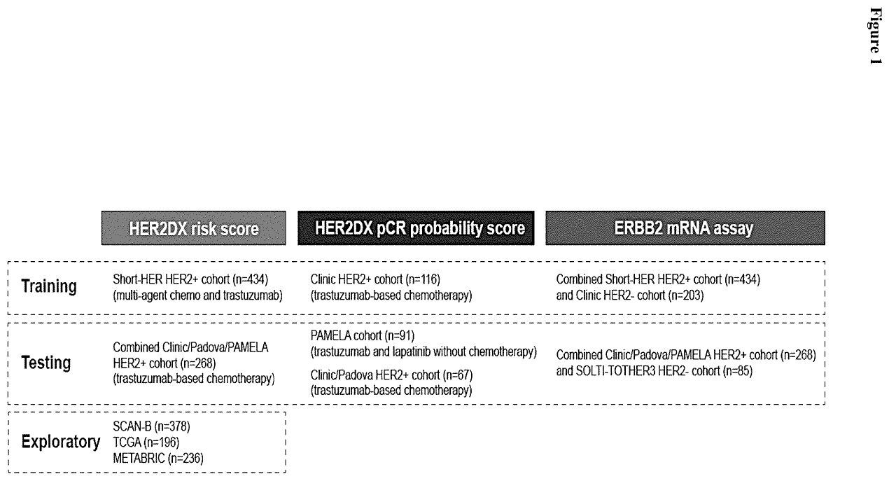

A summary of all the cohorts evaluated is available in FIG. 1. Short-HER was a randomized, multicentric, investigator-driven phase 3 study, aimed to assess the non-inferiority of 9 weeks versus 1 year of adjuvant trastuzumab combined with chemotherapy. Briefly, women aged 18-75 with surgically resected, HER2+ breast cancer, suitable for adjuvant chemotherapy were eligible. Women had to have node positivity, or in case of node-negativity, at least one of the following features: tumor size>2 cm, grade 3, presence of lympho-vascular invasion, Ki67>20%, age<35 years or hormone receptor negativity. Patients with stage IIIB/IV disease were not eligible. A total of 1,254 patients with a performance status of 0-1 were randomized from 17 Dec. 2007 to 6 Oct. 2013 to arm A or arm B. Chemotherapy in arm A (long) consisted of adriamycin 60 mg/m² plus cyclophosphamide 600 mg/m² or epirubicin 90 mg/m² plus cyclophosphamide 600 mg/m² every 3 weeks for 4 courses followed by paclitaxel 175 mg/m² or docetaxel 100 mg/m² every 3 weeks for 4 courses. Trastuzumab was administered every 3 weeks for 18 doses, starting with the first taxane dose. Chemotherapy in arm B (short) consisted of docetaxel 100 mg/m² every 3 weeks for 3 courses followed by 5-fluorouracil 600 mg/m², epirubicin 60 mg/m², cyclophosphamide 600 mg/m² every 3 weeks for 3 courses. Trastuzumab was administered weekly for 9 weeks, starting concomitantly with docetaxel. When indicated, radiation and hormonal therapy were carried out according to local standard. Median follow-up was 98.4 months.

PAMELA was an open-label, single-group, phase 2 trial from 22 Oct. 2013 to 30 Nov. 2015 aimed to the ability of the PAM50 HER2-enriched subtype to predict pCR at the time of surgery. Patients with HER2+ disease, stage I-IIIA and a performance status of 0-1 were given lapatinib (1,000 mg per day) and trastuzumab for 18 weeks; hormone receptor-positive patients were additionally given letrozole (2.5 mg per day) or tamoxifen (20 mg per day) according to menopausal status. Treatment after surgery was left to treating physician discretion. Median follow-up was 68.1 months.

The Hospital Clinic and Padova University HER2-positive cohorts are consecutive series of patients with early-stage HER2+ breast cancer and a performance status of 0-1 treated, as per standard practice, from 28 Jun. 2005 to 26 Sep. 2020 (Hospital Clinic) and 23 Feb. 2009 to 26 May 2016 (Padova University cohort), with neoadjuvant trastuzumab-based multi-agent chemotherapy for 3-6 months, followed by surgery. Adjuvant treatment was completed with trastuzumab for up to 1 year, and a minimum of 5 years of hormonal therapy for patients with hormone receptor-positive tumors. Radiation therapy was administered according to local guidelines. Median follow-up of Hospital Clinic and Padova University cohorts were 43.1 and 49.9 months, respectively.

Three publicly available gene expression-based datasets that included clinical data and survival outcome from patients with HER2-positive early-stage breast cancer were explored. All the data from The Cancer Genome Atlas (TCGA) and METABRIC datasets were obtained from the cbioportal webpage. The data from the SCAN-B dataset was obtained from GEO, under accession number GSE81540. The gene expression data from TCGA and SCAN-B is RNA-sequencing-based, whereas the gene expression data from METABRIC is microarray-based. No clear information regarding the type of locoregional and systemic therapy is available from these datasets, although patients in METABRIC did not receive anti-HER2 therapy.

Finally, we included two cohorts of consecutive patients with newly diagnosed HER2-negative breast cancer from Hospital Clinic and from the SOLTI-1805 TOT-HER3 trial, a window-of-opportunity trial. Only baseline pre-treated tumors were analyzed. No follow-up was available.

The study was performed in accordance with Good Clinical Practice guidelines and the World Medical Association Declaration of Helsinki. Approvals for the study were obtained from independent ethics committees.

### Example 1.2. Tumor Sample Procedures

Gene expression assays were performed on tumor samples from Short-HER, PAMELA, Padova University cohort and Hospital Clinic of Barcelona cohort at the Translational Genomics and Targeted Therapies in Solid Tumors at IDIBAPS. A minimum of −125 ng of total RNA was used to measure the expression of 185 breast cancer-related genes and 5 housekeeping genes (GAPD, PUM1, ACTB, RPLP0 and PSMC4) using the nCounter platform (Nanostring Technologies, Seattle, USA). Finally, TILs in Short-HER were assessed on a single hematoxylin-eosin-stained slide and stromal TILs were scored according to pre-defined criteria.

### Example 1.3. HER2DX Gene Signatures

HER2DX is based on 4 different gene signatures comprising 27 genes, which capture various biological processes, including immune infiltration, tumor cell proliferation, luminal differentiation, and expression of the HER2 amplicon. The immune signature selected for HER2DX was the 14-gene immunoglobulin (IGG) module (i.e., CD27, CD79A, HLA-C, IGJ, IGKC, IGL, IGLV3-25, IL2RG, CXCL8, LAX1, NTN3, PIM2, POU2AF1 and TNFRSF17), previously identified by unsupervised clustering of human breast tumors. The IGG signature has previously shown strong independent prognostic value in a large breast cancer dataset, where patients did not receive adjuvant systemic therapy. The other three gene signatures were identified from unsupervised clustering of the Short-HER HER2-positive dataset using data from 185-breast cancer-related genes. The genes selected were obtained from highly correlated gene clusters (correlation coefficient>0.80); the tumor cell proliferation signature includes 4 genes (i.e., EXO1, ASPM, NEK2 and KIF23), the luminal differentiation signature includes 5 genes (i.e., BCL2, DNAJC12, AGR3, AFF3 and ESR1), and the HER2 amplicon signature includes 4 genes located in the 1711-12 chromosome (i.e., ERBB2, GRB7, STARD3 and TCAP). For each signature, the mean gene expression was calculated for each patient.

### Example 1.4. Outcomes

The co-primary objectives of this study were to derive and validate two independently trained HER2DX scores: a prognostic risk score, and a pCR probability score. In the prognostic training dataset (i.e., Short-HER), the survival endpoint was DRFS, calculated as the time between randomization and distant recurrence or death before recurrence. In the validation prognostic dataset, the survival endpoint was DFS due to the availability of the data, which was calculated as the time between randomization and any of the following events, whichever first: local, regional, and distant recurrence; contralateral breast cancer, excluding in situ carcinoma; other second invasive primary cancer; death before recurrence or second primary cancer. In all neoadjuvant datasets, pCR at surgery was defined as no invasive tumor cells in the breast and axilla.

The secondary objectives were: 1) to describe the clinical-pathological features of the HER2DX risk groups; 2) to explore in-silico the association of HER2DX risk score with overall survival (OS) in publicly available datasets of HER2-positive early-stage breast cancer; 3) to evaluate the value of ERBB2 mRNA to predict HER2 status according to the ASCO/CAP guidelines.

### Example 1.5. HER2DX Risk Score Development and Validation

The 434 patients enrolled in the Short-HER trial were used as the training dataset. Patient samples in the training dataset were split into a training set (67% of samples) and a testing set (remaining 33% of samples), balancing for distant relapse-free survival (DRFS) event and treatment arm. Prognostic models of different feature sets were compared by C-index, the index of rank concordance for survival data. These feature sets were evaluated by Monte-Carlo cross validation (MCCV) with 100 iterations. Cox proportional hazard models were fit with ridge regression or elastic net in each iteration of training and evaluated in the MCCV testing sets.

A single cut-off from the final HER2DX risk score was selected to split patients into low- and high-risk groups. The criteria to select this cut-off was that the low-risk group must have a lower boundary of the 95% confidence interval of the DRFS estimate above 90% at 3, 5 and 7 years. The final HER2DX risk score was tested, as a continuous variable and using the pre-specified cut-off, in 268 patients from the validation dataset. The validation dataset was composed of patients from Hospital Clinic of Barcelona HER2-positive cohort (n=147), PAMELA (n=84) and the Padova University cohort (n=37). The median follow-up of the validation dataset was 51.0 months.

To further evaluate the prognostic value of the HER2DX risk score, the HER2DX algorithm was evaluated in-silico across three publicly available datasets of patients with early-stage HER2-positive breast cancer (i.e., TCGA, METABRIC and SCAN-B). HER2DX risk models with and without clinical variables (i.e., tumor and nodal staging) were explored as continuous variables due to the known technical biases between different genomic platforms.

### Example 1.6. HER2DX pCR Probability Score Development and Validation

One-hundred and sixteen patients with early-stage HER2-positive breast cancer treated with neoadjuvant trastuzumab-based chemotherapy at Hospital Clinic of Barcelona were used as the training dataset for the HER2DX pCR probability score. Patient samples in the training dataset were split into a training set (67% of samples) and a testing set (remaining 33% of samples), balancing for pCR status. Logistic regression models were fit with ridge regression in each iteration of training and evaluated in the MCCV testing sets. Two cut-offs based on tertiles in the training dataset was defined to split patients into three groups: low pCR probability, medium pCR probability and high pCR probability. The final HER2DX pCR probability score was tested, as a continuous variable and using the pre-specified cut-offs, in 158 patients from two validation datasets. The first validation dataset was composed of 67 patients treated with trastuzumab-based chemotherapy from Padova University cohort (n=37) and Hospital Clinic of Barcelona cohort (n=30). The second validation dataset was composed of 91 patients treated with neoadjuvant lapatinib and trastuzumab without chemotherapy from the PAMELA study.

### Example 1.7. HER2DX ERBB2 mRNA Expression Assay

A cohort of 637 patients with primary invasive breast cancer and known HER2 status according to the ASCO/CAP guidelines was evaluated using the HER2DX assay and used as the training dataset to predict clinical HER2 status. This dataset was composed of 203 patients with newly diagnosed early-stage HER2-negative at Hospital Clinic breast cancer and the Short-HER HER2-positive cohort of 434 patients. The optimal cutoff of ERBB2 expression to predict HER2 clinical status (positive versus negative) was obtained from a receiver operation curve and Youden index analysis. The optimal ERBB2 cutoff was validated in an independent cohort of 353 HER2-negative and HER2+ cases from the SOLTI-1805 TOT-HER3 HER2-negative trial (n=85), Hospital Clinic of Barcelona HER2-positive cohort (n=147), PAMELA (n=84) and Padova University cohort (n=37).

### Example 1.8. General Statistical Procedures

For description purposes, 3-, 5- and 7-year estimates of DRFS or DFS were calculated by Kaplan-Meier. Univariate and multivariable Cox proportional hazard regression analyses were used to investigate the association of each variable with survival outcome. To evaluate the prognostic contribution of each variable, likelihood ratio values (χ2) were used to measure and compare the relative amount of prognostic information. Categorical variables were expressed as number (%) and compared by χ² test or Fisher's exact test. Logistic regression analyses were performed to investigate the association of each variable with pCR. C-index and receiver operating characteristic (ROC) curves were used as a performance measure. The significance level was set to a 2-sided alpha of 0.05. We used R version 4.0.5. for all the statistical analyses.

### Example 1.9. Role of the Funding Source

The study was designed and performed by investigators from Padova University, Hospital Clinic and Reveal Genomics. All authors had full access to all data in the study and had final responsibility for the decision to submit for publication.

### Example 2. Results

### Example 2.1. HER2DX Risk Score Development and Validation

To build a prognostic model, clinical-pathological and gene expression data were available from 434 (35%) of 1,254 patients in the Short-HER trial (Table 1).

TABLE 1

| | | | HER2DX | | HER2DX | | |
|---|---|---|---|---|---|---|---|
| | All patients | | Low-Risk | | High-Risk | | |
| | N | % | N | % | N | % | p-value* |
| N | 434 | — | 216 | 49.8% | 218 | 50.2% | — |
| Age (mean) | 55.4 | | 55.6 | | 55.1 | | 0.580 |
| TILs | | | | | | | |
| TILs 0-29 | 378 | 87.1% | 178 | 82.4% | 200 | 91.7% | 0.004 |
| TILs ≥30 | 56 | 12.9% | 38 | 17.6% | 18 | 8.3% | |
| pT | | | | | | | |
| T1 | 234 | 53.9% | 152 | 70.4% | 82 | 37.6% | <0.001 |
| T2 | 187 | 43.1% | 63 | 29.2% | 124 | 56.9% | |
| T3-4 | 13 | 3.0% | 1 | 0.4% | 12 | 5.5% | |
| pN | | | | | | | |
| N0 | 235 | 54.2% | 208 | 96.3% | 27 | 12.4% | <0.001 |
| N1 | 134 | 30.8% | 8 | 3.7% | 126 | 57.8% | |
| N2-3 | 65 | 15.0% | 0 | 0.0% | 65 | 29.8% | |
| Estrogen receptor | | | | | | | |
| status | | | | | | | |
| Positive | 321 | 74.0% | 155 | 71.8% | 166 | 76.1% | 0.326 |
| Negative | 113 | 26.0% | 61 | 28.2% | 52 | 23.9% | |
| Treatment arm | | | | | | | |
| Arm A (long) | 221 | 50.9% | 112 | 51.2% | 109 | 50.0% | 0.702 |
| Arm B (short) | 213 | 49.1% | 104 | 48.2% | 109 | 50.0% | |
| Grade | | | | | | | |
| Grade 1 | 6 | 1.4% | 0 | 0.0% | 6 | 2.8% | 0.334 |
| Grade 2 | 115 | 26.8% | 65 | 30.5% | 50 | 23.1% | |
| Grade 3 | 308 | 71.8% | 148 | 69.5% | 160 | 74.1% | |
| Intrinsic subtype | | | | | | | |
| Luminal A | 128 | 29.5% | 65 | 30.1% | 63 | 28.9% | 0.008 |
| Luminal B | 36 | 8.3% | 10 | 4.6% | 26 | 11.9% | |
| HER2-enriched | 213 | 49.1% | 104 | 48.2% | 109 | 50.0% | |
| Basal-like | 25 | 5.7% | 14 | 6.5% | 11 | 5.0% | |
| Normal-like | 32 | 7.4% | 23 | 10.6% | 9 | 4.1% | |

Patient baseline characteristics of the Short-HER dataset.
TILs: tumour-infiltrating lymphocytes;
*p-values represent comparison between HERDX low-risk and high-risk groups.

Mean age was 55.4 and most tumors were 2 cm or less (T1 stage), node-negative (NO stage), hormone receptor-positive and histological grade 3. In this cohort, our previous study showed that the best prognostic models integrated tumor size, nodal status, TILs, and the main biology associated with the 4 intrinsic subtypes. Based on these previous findings, we re-develop HER2DX risk score based on 4 gene expression-based signatures tracking immune infiltration, tumor cell proliferation, HER2 amplicon expression and tumor cell luminal differentiation, together with tumor stage (T1 vs. T2 vs. T3-4) and nodal stage (NO vs. N1 vs. N2-3). To capture immune infiltration, we selected our previously described IGG signature, which has shown a strong prognostic value in early-stage breast cancer. HER2DX variables were associated with good outcome (i.e., immune/IGG, and luminal) and poor outcome (i.e., proliferation, and tumor and nodal staging) when tested in univariate analyses. Overall, the predictive performance (C-index) of the HER2DX risk score in Short-HER was 0.74, which was very similar (0.72) to the C-index of our previously reported HER2DX risk model based on 17 different variables. Of note, when we tried to add more variables into the current HER2DX risk model, including TILs, intrinsic subtypes, and individual genes, the predictive performance of HER2DX did not improve.

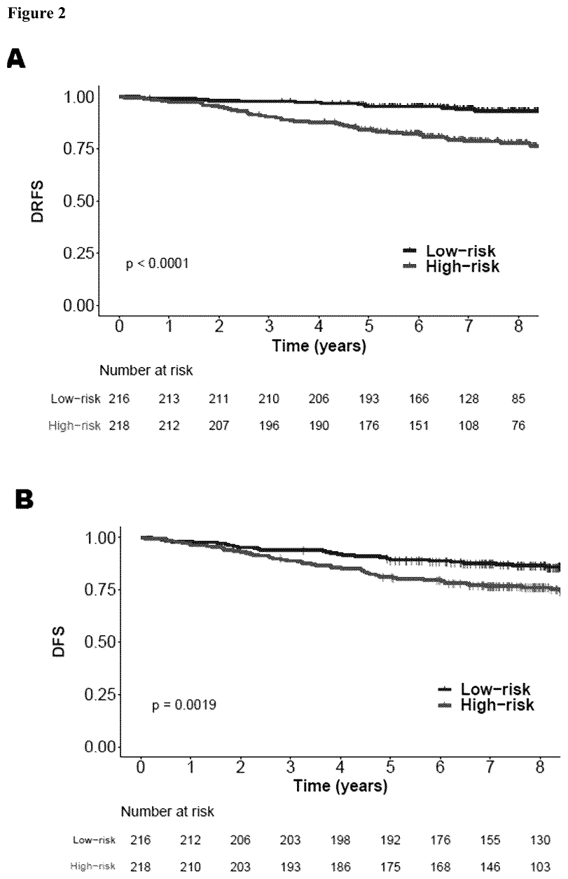

HER2DX measured as a continuous variable was significantly associated with distant relapse-free survival (DRFS) in the Short-HER 434 patient-dataset (p<0.001). To select a clinically relevant cutoff, we defined low-risk as a group of patients with a 3-, 5- and 7-year DRFS with a lower boundary of the 95% confidence interval (CI)>90%. This selected cutoff identified 49.8% of patients (n=216) as low risk. The 3-, 5- and 7-year DRFS of the low-risk population was 97.7% (95% CI 95.7-99.7), 95.3% (95% CI 92.5-98.2) and 94.0% (95% CI 90.6-97.4), respectively (FIG. 2A). The 3-, 5- and 7-year DRFS of the high-risk population was 90.4% (95% CI 86.5-94.4), 84.3% (95% CI 79.6-89.3) and 78.6% (95% CI 73.2-84.5), respectively. The DRFS, DFS and OS hazard ratios (HRs) between the low- and high-risk groups were 0.26 (95% CI 0.1-0.5), 0.51 (95% CI 0.3-0.8) and 0.45 (95% CI 0.2-0.9), respectively (FIG. 2A-C). In terms of clinical-pathological characteristics, the two risk-groups showed statistically significant differences in terms of TILs, nodal status, tumor size, and intrinsic subtype (Table 1).

A dataset of 268 patients with early-stage HER2-positive disease obtained from a combined cohort of three neoadjuvant studies was used for an independent evaluation of the HER2DX score (the score was determined on pretreatment specimens before starting neoadjuvant therapy; Table 2).

TABLE 2

| | | | HER2DX | | HER2DX | | |
|---|---|---|---|---|---|---|---|
| | All patients | | Low Risk | | High Risk | | |
| | N | % | N | % | N | % | p-value* |
| N | 268 | — | 136 | 50.7% | 132 | 49.3% | |
| Age (mean) | 56.3 | | 56.2 | | 56.3 | | 0.980 |
| TILs | | | | | | | |
| TILs 0-29 | 220 | 85.3% | 112 | 84.8% | 108 | 85.7% | 0.984 |
| TILs ≥30 | 38 | 14.7% | 20 | 15.2% | 18 | 14.3% | |
| Clinical tumor stage | | | | | | | |
| T1 | 84 | 21.3% | 61 | 45.0% | 23 | 17.4% | <0.001 |
| T2-I | 184 | 78.7% | 75 | 55.0% | 109 | 82.6% | |
| Clinical nodal stage | | | | | | | |
| N0 | 162 | 55.4% | 136 | 100.0% | 26 | 20.0% | <0.001 |
| N1-3 | 106 | 44.6% | 0 | 0% | 106 | 80.0% | |
| Pathological response | | | | | | | |
| pCR | 118 | 44.0% | 58 | 42.6% | 60 | 45.5% | 0.734 |
| Residual disease | 150 | 56.0% | 78 | 57.4% | 72 | 54.5% | |
| Hormone receptor | | | | | | | |
| status | | | | | | | |
| Positive | 171 | 63.8% | 96 | 70.6% | 75 | 56.8% | 0.027 |
| Negative | 97 | 36.2% | 40 | 29.4% | 57 | 43.2% | |
| Intrinsic subtype | | | | | | | |
| Luminal A | 43 | 19.1% | 30 | 22.1% | 13 | 9.8% | 0.003 |
| Luminal B | 30 | 12.4% | 15 | 11.0% | 15 | 11.4% | |
| HER2-enriched | 158 | 51.7% | 67 | 49.2% | 91 | 69.0% | |
| Basal-like | 16 | 7.9% | 8 | 5.9% | 8 | 6.0% | |
| Normal-like | 21 | 9.0% | 16 | 11.8% | 5 | 3.8% | |
| Study | | | | | | | |
| PAMELA | 84 | 31.3% | 46 | 33.8% | 38 | 28.8% | 0.673 |
| HOSPITAL | 147 | 54.9% | 72 | 53.0% | 75 | 56.8% | |
| CLINIC | | | | | | | |
| PADOVA | 37 | 13.8% | 18 | 13.2% | 19 | 14.4% | |

Patient baseline characteristics of the combined prognostic validation dataset.
TILs: tumour-infiltrating lymphocytes;
pCR: pathological complete response;
*p-values represent comparison between HERDX low-risk and high-risk groups.

The evaluation dataset was composed of 147 patients from Hospital Clinic, 84 (56%) of 151 from PAMELA and 37 from the Padova University cohort. All patients received chemotherapy and 1 year of trastuzumab; 84 (31%) of 268 patients received dual HER2 blockade with lapatinib and trastuzumab for 4.5 to 6.0 months, and 66 (25%) of 268 received four to six cycles of neoadjuvant pertuzumab. Despite heterogeneity in systemic therapies, there were no significant differences in DFS across the four cohorts, or between patients treated with trastuzumab-only versus dual HER2 blockade.

In the independent prognostic dataset, HER2DX score as a continuous variable was significantly associated with DFS (HR 1.03, 95% CI 1.0-1.1, p=0.002). In this dataset, for every 10-unit increase (from 0 to 100) in HER2DX risk score, there was a 30% increase in the hazard for the event. According to the prespecified cutoffs, the HER2DX low-risk group had longer DFS than the high-risk (HR 0.21, 95% CI 0.1-0.6, p-value=0.005) (FIG. 2B). 5-year DFS in the HER2DX low-risk and high-risk groups was 95.3% (95% CI 92.4-98.2) and 84.0% (79.6-89.3), respectively. 7-year DFS in the HER2DX low-risk and high-risk groups was 93.9% (95% CI 90.6-97.4) and 78.6% (73.2-84.5), respectively. The C-index of the HER2DX risk score was 0.73 for all patients.

To further explore the prognostic value of the HER2DX risk score, we interrogated three publicly available breast cancer datasets (i.e., TCGA, METABRIC and SCAN-B), which include clinical data, overall survival (OS) outcome and gene expression data for a total of 810 patients with early-stage HER2-positive breast cancer. The HER2DX algorithm was applied in each dataset with and without clinical features (i.e., tumor and nodal staging) (Table 3).

TABLE 3
Table 3. Association of the HER2DX risk score* with overall
survival across three publicly available datasets.

| | HR | 95% CI | p-value | χ2 |
|---|---|---|---|---|
| SCAN-B (n = 378) | | | | |
| HER2DX risk score (GEP) | 5.0 | 2.4-10.6 | <0.001 | 18.7 |
| HER2DX risk score | 2.8 | 1.9-4.1 | <0.001 | 31.9 |
| (GEP + Clinical) | | | | |
| TCGA (n = 196) | | | | |
| HER2DX risk score (GEP) | 5.8 | 2.4-13.8 | <0.001 | 15.6 |
| HER2DX risk score | 4.0 | 1.8-8.6 | 0.001 | 15.4 |
| (GEP + Clinical) | | | | |
| METABRIC (n = 236) | | | | |
| HER2DX risk score (GEP) | 2.2 | 1.2-3.7 | 0.007 | 7.31 |
| HER2DX risk score | 1.7 | 1.3-2.1 | <0.001 | 22.0 |
| (GEP + Clinical) | | | | |

*HER2DX risk score was evaluated using the 4 gene expression-based variables (GEP), and the full HER2DX risk core which includes tumor and nodal staging (GEP + Clinical). To evaluate the prognostic contribution of each score, likelihood ratio values (χ2) were used to measure and compare the relative amount of prognostic information. HR, hazard ratio; CI, confidence interval. SCAN-B dataset (source: GSE81540); The Cancer Genome Atlas (TCGA) dataset (source: cbioportal.org/); METABRIC dataset (source: cbioportal.org/).

A statistically significant association between HER2DX risk score as a continuous variable and OS was observed across the tested public datasets. Overall, these in-silico results support the strong prognostic value of HER2DX.

### Example 2.2. HER2DX pCR Probability Score Development and Validation

To build a predictive model, we evaluated the HER2DX assay in pre-treated tumors from 120 patients with early-stage HER2-positive breast cancer treated with neoadjuvant trastuzumab-based chemotherapy (Table 4).

TABLE 4

| | Validation cohorts | | | | | |
|---|---|---|---|---|---|---|
| | Training cohort | | PAMELA | | Clinic/Padova | |
| | N | % | N | % | N | % |
| N | 116 | — | 91 | — | 67 | — |
| Chemotherapy backbone | 116 | 100% | 0 | 0% | 67 | 100% |
| Anti-HER2 therapy | | | | | | |
| Trastuzumab-only | 69 | 59.5% | 0 | 0.0% | 48 | 71.6% |
| Trastuzumab and lapatinib | 0 | 0.0% | 91 | 100.0% | 0 | 0.0% |
| Trastuzumab and | 47 | 40.5% | 0 | 0.0% | 19 | 28.4% |
| pertuzumab | | | | | | |
| Age (mean) | 57.3 | | 56.0 | | 56.2 | |
| TILs | | | | | | |
| TILs 0-29 | 98 | 86.0% | 75 | 82.4% | 52 | 88.1% |
| TILs ≥30 | 16 | 14.0% | 16 | 17.6% | 7 | 11.9% |
| Clinical tumor stage | | | | | | |
| T1 | 32 | 27.6% | 36 | 39.6% | 17 | 25.4% |
| T2-4 | 84 | 72.4% | 55 | 60.4% | 50 | 74.6% |
| Clinical nodal stage | | | | | | |
| N0 | 65 | 56.0% | 54 | 59.3% | 45 | 67.2% |
| N1-3 | 51 | 44.0% | 37 | 40.7% | 22 | 32.8% |
| Pathological response | | | | | | |
| pCR | 60 | 51.7% | 32 | 35.2% | 30 | 44.8% |
| Residual disease | 56 | 48.3% | 59 | 64.8% | 37 | 55.2% |
| Hormone receptor status | | | | | | |
| Positive | 79 | 68.1% | 49 | 53.8% | 48 | 71.6% |
| Negative | 37 | 31.9% | 42 | 46.2% | 19 | 28.4% |
| Intrinsic subtype | | | | | | |
| Luminal A | 24 | 20.7% | 10 | 11.0% | 9 | 13.4% |
| Luminal B | 10 | 8.6% | 8 | 8.8% | 13 | 19.4% |
| HER2-enriched | 66 | 56.9% | 62 | 68.1% | 35 | 52.2% |
| Basal-like | 8 | 6.9% | 6 | 6.6% | 2 | 3.0% |
| Normal-like | 8 | 6.9% | 5 | 5.5% | 8 | 12.0% |

Patient characteristics of the training and validation neoadjuvant datasets.
TILs: tumour-infiltrating lymphocytes;
pCR: pathological complete response.

Mean age was 55.4 (SD 10.2) and most tumors were 2 cm or less (T1 stage), node-negative (NO stage), hormone receptor-positive and histological grade 3. The 4 gene signatures (i.e., HER2 amplicon, immune/IGG, luminal and proliferation) and the 2 clinical variables (i.e., tumor and nodal staging) were used to train a HER2DX pCR probability score. HER2DX variables were associated with pCR (i.e., immune/IGG, and proliferation) and non-pCR (i.e., luminal, and tumor and nodal staging). Overall, the predictive performance (AUC) of the HER2DX pCR probability score in the training dataset was 0.81.

Two cohorts of 97 and 67 patients with early-stage HER2-positive disease treated with neoadjuvant anti-HER2-based therapy was used for an independent validation of the HER2DX pCR probability score (the score was determined at baseline before starting neoadjuvant therapy; Table 5).

TABLE 5

| | HER2DX pCR probability score* | | | | | | |
|---|---|---|---|---|---|---|---|
| | Low | | Medium | | High | | |
| | N | % | N | % | N | % | P-value |
| N | 88 | — | 83 | — | 103 | — | |
| Chemotherapy backbone | 64 | 72.7% | 58 | 69.9% | 61 | 59.2% | 0.110 |
| AntiHER2 therapy | | | | | | | |
| Trastuzumab-only | 38 | 43.2% | 39 | 47.0% | 40 | 38.8% | 0.249 |
| Trastuzumab and lapatinib | 24 | 27.3% | 25 | 30.1% | 42 | 40.8% | |
| Trastuzumab and | 26 | 29.5% | 19 | 22.9% | 21 | 20.4% | |
| pertuzumab | | | | | | | |
| Age (mean) | 56.5 | | 53.2 | | 58.2 | | |
| TILs | | | | | | | |
| TILs 0-29 | 77 | 92.8% | 73 | 90.1% | 75 | 75.0% | 0.001 |
| TILs ≥30 | 6 | 7.2% | 8 | 9.9% | 25 | 25.9% | |
| Clinical tumor stage | | | | | | | |
| T1 | 21 | 23.9% | 23 | 27.7% | 41 | 39.8% | 0.044 |
| T2-4 | 67 | 76.1% | 60 | 72.3% | 62 | 60.2% | |
| Clinical nodal stage | | | | | | | |
| N0 | 57 | 64.8% | 46 | 55.4% | 61 | 59.2% | 0.453 |
| N1-3 | 31 | 35.2% | 37 | 44.6% | 42 | 40.8% | |
| Hormone receptor status | | | | | | | |
| Positive | 82 | 93.2% | 58 | 69.9% | 36 | 35.0% | <0.001 |
| Negative | 6 | 6.8% | 25 | 30.1% | 67 | 65.0% | |
| Intrinsic subtype | | | | | | | |
| Luminal A | 37 | 42.1% | 5 | 6.0% | 1 | 1.0% | <0.001 |
| Luminal B | 18 | 20.5% | 10 | 12.1% | 3 | 2.9% | |
| HER2 -enriched | 28 | 31.8% | 56 | 67.5% | 79 | 76.7% | |
| Basal-like | 1 | 1.1% | 1 | 1.2% | 14 | 13.6% | |
| Normal-like | 4 | 4.5% | 11 | 13.2% | 6 | 5.8% | |

Patient characteristics of the training and validation neoadjuvant datasets combined according to HER2DX pCR probability score.
*Groups using the pre-specified cutoffs are shown.
TILs: tumour-infiltrating lymphocytes.

In both cohorts, HER2DX pCR probability score as a continuous variable was found statistically significantly associated with pCR (p<0.001). Overall, the predictive performances (AUC) of the HER2DX pCR probability score in the PAMELA study and the trastuzumab-based chemotherapy cohort were 0.80 and 0.77, respectively. As expected, statistically significant differences in pCR rates across the three response groups (i.e., defined by tertiles, which were determined in the training dataset), were observed (Table 6).

TABLE 6

| | Low Medium | | Medium | | High | | |
|---|---|---|---|---|---|---|---|
| | N | % | N | % | N | % | P-value |
| pCR rates in cohort 1* | 6/26 | 23.1% | 8/19 | 42.1% | 16/22 | 72.7% | 0.003 |
| pCR rates in cohort 2* | 2/24 | 8.3% | 4/25 | 16.0% | 26/42 | 61.9% | <0.001 |

pCR rates across the two validation neoadjuvant datasets according to HER2DX pCR probability score.
*Validation cohort 1 includes 67 patients treated with trastuzumab-based chemotherapy. Validation cohort 2 includes 91 patients who participated in the PAMELA trial. Groups using the pre-specified cut-offs are shown.

### Example 2.3. Relationships Between Both HER2DX Scores

To determine the similarity (or lack thereof) between both HER2DX scores, we evaluated a combined HER2-positive dataset that included Short-HER (n=434) and the validation prognostic dataset (n=268). Overall, the correlation coefficient of both HER2DX scores was weak (i.e., −0.19). In patients with HER2DX low-risk, 46.3% (163/352) were identified as HER2DX high probability of pCR and 53.7% (189/352) as HER2DX low/med probability of pCR. In patients with HER2DX high-risk, 33.1% (116/350) were identified as having a HER2DX high probability of pCR and 66.9% (234/350) as having a HER2DX low/med probability of pCR.

### Example 2.4. HER2DX ERBB2 mRNA Expression Assay

ERBB2 mRNA expression within HER2-positive breast cancer can help identify patients with a high response to anti-HER2 therapies, including T-DM1. In addition, ERBB2 mRNA expression can help identify HER2 status according to the ASCO/CAP guidelines. To build an ERBB2 mRNA expression assay that tracks with clinical HER2 status, we combined the Short-HER HER2-positive cohort (n=434) with a HER2-negative cohort of patients newly diagnosed of early-stage breast cancer at Hospital Clinic (n=203). Overall, the mean ERBB2 expression (in log base 2) in HER2-negative and HER2-positive disease was −2.01 and 1.24, respectively (a 6.5-fold difference). The ROC AUC of ERBB2 expression to predict clinical HER2 status was 0.97 with a 90% sensitivity and 98% specificity. Using Youden's analysis, an optimal cutoff of −0.98 was identified. 3.4% of clinically defined HER2-negative cases were identified as ERBB2-positive by mRNA, and 9.7% of clinically defined HER2-positive cases were identified as ERBB2-negative/low.

The optimal cutoff to predict HER2 status was tested in an independent dataset of 85 HER2-negative and 268 HER2-positive cases (FIG. 1). Overall, the mean ERBB2 expression (in log base 2) in HER2-negative and HER2-positive disease was −2.17 and 0.96, respectively (a 6.3-fold difference). The ROC AUC of ERBB2 expression to predict clinical HER2 status was 0.96 with an 84% sensitivity and 100% specificity. No HER2-negative cases were identified as ERBB2-positive, and 16.4% of HER2-positive cases were identified as ERBB2-negative/low.

### Example 2.5. Interaction Between 4 Individual Genes (as a Continuous Variable) and Treatment Arm (9 Weeks vs 1-Year) in Terms of DMFS at 96 Months

A total of 4 genes (i.e., CD86, FA2H, FGFR2 and ERBB3) were found associated with trastuzumab benefit in terms of DMFS according to treatment duration (i.e., 1-year versus 9-weeks). Low CD86 expression (as a continuous variable) was found associated with more benefit if patients are treated for 1-year compared to 9-weeks (CD86*Arm, 9 weeks trastuzumab treatment versus 1-year, Hazard Ratio=0.350, interaction p-value=0.0017). Low FA2H expression (as a continuous variable) was found associated with more benefit if patients are treated for 1-year compared to 9-weeks (FA2H*Arm, 9 weeks trastuzumab treatment versus 1-year, Hazard Ratio=0.65, interaction p-value=0.046). High FGFR2 expression (as a continuous variable) was found associated with more benefit if patients are treated for 1-year compared to 9-weeks (FGFR2*Arm, 9 weeks trastuzumab treatment versus 1-year, Hazard Ratio=1.68, interaction p-value=0.027). Finally, high ERBB3 expression (as a continuous variable) was found associated with more benefit if patients are treated for 1-year compared to 9-weeks (ERBB3*Arm, 9 weeks trastuzumab treatment versus 1-year, DMFS96 Hazard Ratio=1.99, interaction p-value=0.035).

### Example 2.6. Combinations of at least 2 genes tracking the luminal, proliferation and immune pathways is prognostic in early-stage HER2+ breast cancer

The HER2DX risk score of the HER2DX assay consists of 23 genes [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and ESR1] and it is used to predict prognosis in patients with HER2-positive (HER2+) breast cancer. The 23 genes are part of one of the following 3 gene expression signatures: luminal differentiation signature (n=5 genes), tumor cell proliferation (n=4) and immune signature (n=14).

We evaluated the prognostic value of gene pairs (i.e., combination of 2 genes) included in the 3 signatures across 5 different datasets of patients with early-stage HER2+ breast cancer, including:
1) Short-HER dataset using distant-metastasis free survival (DMFS) as the survival endpoint: 434 patients with HER2+ breast cancer treated with adjuvant anti-HER2 therapy in the context of the Short-HER phase III clinical trial.
2) Short-HER dataset using overall survival (OS) as the endpoint: 434 patients with HER2+ breast cancer treated with adjuvant anti-HER2 therapy in the context of the Short-HER phase III clinical trial.
3) TCGA dataset using OS as the endpoint: 164 patients with HER2+ breast cancer.
4) METABRIC dataset using the OS as the endpoint: 236 patients with HER2+ breast cancer.
5) SCAN-B dataset using the OS as the endpoint: 378 patients with HER2+ breast cancer.

For each pair of genes, a combination score was determined by calculating the ratio of the expression of the 2 genes, as follows:

Combination score=gene 1 mRNA level(log 2 value)−gene 2 mRNA level(log 2 value)

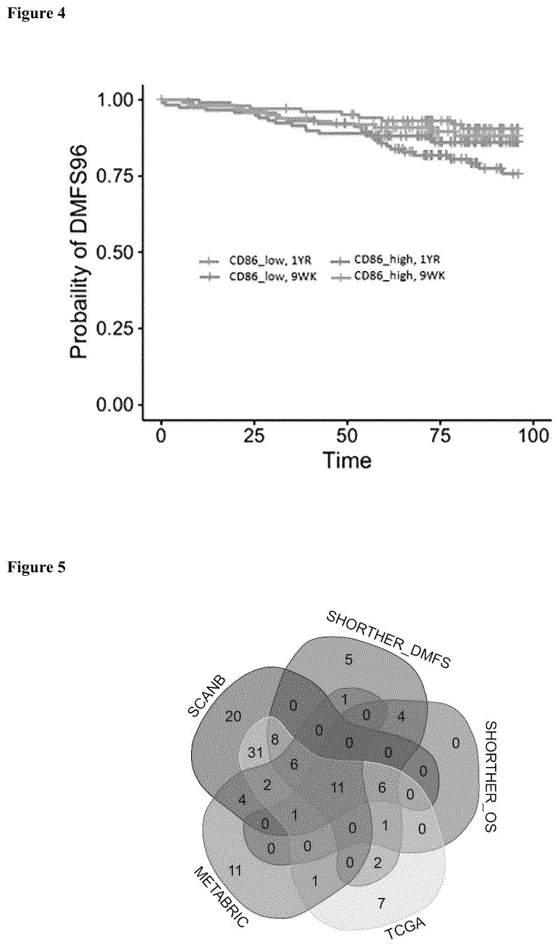

Univariate Cox models for DMFS and OS were used to test the prognostic significance of each combination score. As proof of concept, we identified several pairs significantly associated with prognosis in 2 or more datasets (FIG. 5 and Table 7).

TABLE 7A
List of 78 combination scores significantly associated with good survival outcome in 2 or more datasets

| | | | | | | 95% CI | 95% CI | |
|---|---|---|---|---|---|---|---|---|
| | Gene | | | n | Hazard | lower | higher | |
| Signatures | combination | Dataset | n | events | Ratio | limit | limit | p-value |
| IGG_IGG | CD79A_CD27 | SCANB | 378 | 46 | 0.721 | 0.553 | 0.94 | 0.01548 |
| | | SHORTHER_DMFS | 434 | 63 | 0.712 | 0.558 | 0.909 | 0.00635 |
| | | SHORTHER_OS | 434 | 87 | 0.776 | 0.625 | 0.963 | 0.02148 |
| | | TCGA | 164 | 23 | 0.594 | 0.421 | 0.837 | 0.0029 |
| IGG_IGG | CD27_CXCL8 | SCANB | 378 | 46 | 0.705 | 0.533 | 0.932 | 0.01399 |
| | | TCGA | 164 | 23 | 0.63 | 0.423 | 0.938 | 0.0229 |
| IGG_IGG | CD79A_CXCL8 | SCANB | 378 | 46 | 0.656 | 0.496 | 0.868 | 0.00318 |
| | | SHORTHER_DMFS | 434 | 63 | 0.752 | 0.584 | 0.968 | 0.02688 |
| | | SHORTHER_OS | 434 | 87 | 0.798 | 0.641 | 0.993 | 0.04298 |
| | | TCGA | 164 | 23 | 0.574 | 0.397 | 0.829 | 0.00309 |
| IGG_IGG | IGJ_CXCL8 | SCANB | 378 | 46 | 0.664 | 0.51 | 0.865 | 0.00238 |
| | | TCGA | 164 | 23 | 0.588 | 0.395 | 0.873 | 0.00852 |
| IGG_IGG | POU2AF1_CXCL8 | SCANB | 378 | 46 | 0.643 | 0.487 | 0.848 | 0.00179 |
| | | TCGA | 164 | 23 | 0.667 | 0.46 | 0.965 | 0.03173 |
| IGG_IGG | TNFRSF17_CXCL8 | SCANB | 378 | 46 | 0.623 | 0.47 | 0.825 | 0.00096 |
| | | TCGA | 164 | 23 | 0.618 | 0.423 | 0.903 | 0.01287 |
| IGG_IGG | CD27_HLA.C | SCANB | 378 | 46 | 0.62 | 0.475 | 0.807 | 0.00039 |
| | | TCGA | 164 | 23 | 0.41 | 0.254 | 0.663 | 0.00027 |
| IGG_IGG | CD79A_HLA.C | SCANB | 378 | 46 | 0.62 | 0.474 | 0.811 | 0.00049 |
| | | SHORTHER_DMFS | 434 | 63 | 0.735 | 0.581 | 0.931 | 0.01059 |
| | | TCGA | 164 | 23 | 0.425 | 0.278 | 0.649 | 0.00008 |
| IGG_IGG | IGJ_HLA.C | SCANB | 378 | 46 | 0.647 | 0.501 | 0.835 | 0.00084 |
| | | TCGA | 164 | 23 | 0.481 | 0.31 | 0.745 | 0.00106 |
| IGG_IGG | IL2RG_HLA.C | SCANB | 378 | 46 | 0.646 | 0.496 | 0.841 | 0.00115 |
| | | TCGA | 164 | 23 | 0.49 | 0.301 | 0.798 | 0.00417 |
| IGG_IGG | PIM2 HLA.C | SCANB | 378 | 46 | 0.597 | 0.438 | 0.813 | 0.00105 |
| | | TCGA | 164 | 23 | 0.605 | 0.385 | 0.949 | 0.02882 |
| IGG_IGG | POU2AF1_HLA.C | SCANB | 378 | 46 | 0.59 | 0.448 | 0.778 | 0.00019 |
| | | TCGA | 164 | 23 | 0.561 | 0.367 | 0.858 | 0.00767 |
| IGG_IGG | TNFRSF17_HLA.C | SCANB | 378 | 46 | 0.58 | 0.44 | 0.763 | 0.0001 |
| | | TCGA | 164 | 23 | 0.504 | 0.327 | 0.777 | 0.00192 |
| IGG_IGG | IGL_IGLV3.25 | SHORTHER_DMFS | 434 | 63 | 0.715 | 0.572 | 0.894 | 0.00325 |
| | | SHORTHER_OS | 434 | 87 | 0.803 | 0.659 | 0.979 | 0.02967 |
| IGG_IGG | CD79A_IL2RG | SCANB | 378 | 46 | 0.695 | 0.53 | 0.91 | 0.00823 |
| | | SHORTHER_DMFS | 434 | 63 | 0.694 | 0.535 | 0.9 | 0.00588 |
| | | SHORTHER_OS | 434 | 87 | 0.754 | 0.601 | 0.947 | 0.01508 |
| | | TCGA | 164 | 23 | 0.525 | 0.366 | 0.753 | 0.00047 |
| IGG_IGG | IGJ_IL2RG | SCANB | 378 | 46 | 0.747 | 0.565 | 0.989 | 0.0414 |
| | | TCGA | 164 | 23 | 0.591 | 0.385 | 0.908 | 0.01638 |
| IGG_IGG | TNFRSF17_IL2RG | SCANB | 378 | 46 | 0.672 | 0.508 | 0.889 | 0.0053 |
| | | TCGA | 164 | 23 | 0.631 | 0.416 | 0.957 | 0.03039 |
| IGG_IGG | CD79A_LAX1 | SHORTHER_DMFS | 434 | 63 | 0.733 | 0.583 | 0.922 | 0.00786 |
| | | SHORTHER_OS | 434 | 87 | 0.768 | 0.631 | 0.935 | 0.00853 |
| | | TCGA | 164 | 23 | 0.538 | 0.383 | 0.756 | 0.00036 |
| IGG_IGG | POU2AF1_LAX1 | SHORTHER_DMFS | 434 | 63 | 0.772 | 0.607 | 0.98 | 0.03362 |
| | | SHORTHER_OS | 434 | 87 | 0.758 | 0.617 | 0.931 | 0.00817 |
| IGG_IGG | TNFRSF17_LAX1 | SCANB | 378 | 46 | 0.763 | 0.583 | 0.997 | 0.04779 |
| | | TCGA | 164 | 23 | 0.565 | 0.365 | 0.874 | 0.01038 |
| IGG_IGG | CD27_NTN3 | SCANB | 378 | 46 | 0.717 | 0.541 | 0.949 | 0.01992 |
| | | TCGA | 164 | 23 | 0.51 | 0.316 | 0.822 | 0.00571 |
| IGG_IGG | CD79A_NTN3 | METABRIC | 236 | 147 | 0.819 | 0.692 | 0.969 | 0.02015 |
| | | SCANB | 378 | 46 | 0.679 | 0.515 | 0.893 | 0.00574 |
| | | SHORTHER_DMFS | 434 | 63 | 0.746 | 0.577 | 0.965 | 0.02556 |
| | | SHORTHER_OS | 434 | 87 | 0.782 | 0.628 | 0.975 | 0.02862 |
| | | TCGA | 164 | 23 | 0.476 | 0.311 | 0.728 | 0.00061 |
| IGG_IGG | IGJ_NTN3 | SCANB | 378 | 46 | 0.685 | 0.524 | 0.897 | 0.00587 |
| | | TCGA | 164 | 23 | 0.531 | 0.358 | 0.788 | 0.00167 |
| IGG_IGG | IL2RG_NTN3 | SCANB | 378 | 46 | 0.744 | 0.559 | 0.99 | 0.04235 |
| | | TCGA | 164 | 23 | 0.6 | 0.376 | 0.959 | 0.03276 |
| IGG_IGG | PIM2_NTN3 | METABRIC | 236 | 147 | 0.83 | 0.699 | 0.985 | 0.03343 |
| | | SCANB | 378 | 46 | 0.742 | 0.558 | 0.987 | 0.04032 |
| IGG_IGG | POU2AF1_NTN3 | METABRIC | 236 | 147 | 0.823 | 0.695 | 0.975 | 0.02429 |
| | | SCANB | 378 | 46 | 0.665 | 0.504 | 0.877 | 0.00384 |
| | | SHORTHER_OS | 434 | 87 | 0.8 | 0.64 | 0.999 | 0.04933 |
| | | TCGA | 164 | 23 | 0.605 | 0.405 | 0.904 | 0.0141 |
| IGG_IGG | TNFRSF17_NTN3 | METABRIC | 236 | 147 | 0.842 | 0.71 | 0.998 | 0.0479 |
| | | SCANB | 378 | 46 | 0.648 | 0.491 | 0.853 | 0.00203 |
| | | TCGA | 164 | 23 | 0.532 | 0.342 | 0.828 | 0.00515 |
| IGG_IGG | CD79A_PIM2 | SCANB | 378 | 46 | 0.716 | 0.556 | 0.921 | 0.00938 |
| | | SHORTHER_DMFS | 434 | 63 | 0.72 | 0.573 | 0.905 | 0.00493 |
| | | SHORTHER_OS | 434 | 87 | 0.822 | 0.676 | 0.998 | 0.04802 |
| | | TCGA | 164 | 23 | 0.504 | 0.353 | 0.719 | 0.00016 |
| IGG_IGG | IGJ_PIM2 | SCANB | 378 | 46 | 0.734 | 0.563 | 0.957 | 0.02213 |
| | | TCGA | 164 | 23 | 0.58 | 0.397 | 0.848 | 0.00494 |
| IGG_IGG | POU2AF1_PIM2 | SCANB | 378 | 46 | 0.684 | 0.534 | 0.875 | 0.00256 |
| | | TCGA | 164 | 23 | 0.675 | 0.469 | 0.971 | 0.03418 |
| IGG_IGG | TNFRSF17_PIM2 | SCANB | 378 | 46 | 0.659 | 0.512 | 0.847 | 0.00112 |
| | | TCGA | 164 | 23 | 0.597 | 0.409 | 0.872 | 0.00762 |
| IGG_IGG | CD79A_POU2AF1 | SHORTHER_DMFS | 434 | 63 | 0.787 | 0.626 | 0.989 | 0.03955 |
| | | TCGA | 164 | 23 | 0.602 | 0.458 | 0.791 | 0.00027 |
| IGG_LUM | HLA.C_AGR3 | SCANB | 378 | 46 | 0.746 | 0.569 | 0.977 | 0.03305 |
| | | METABRIC | 236 | 147 | 0.842 | 0.711 | 0.998 | 0.04755 |
| IGG_LUM | CD79A_AGR3 | METABRIC | 236 | 147 | 0.817 | 0.697 | 0.958 | 0.01307 |
| | | SHORTHER_DMFS | 434 | 63 | 0.772 | 0.598 | 0.996 | 0.04676 |
| IGG_LUM | CD79A_BCL2 | SHORTHER_DMFS | 434 | 63 | 0.785 | 0.617 | 0.998 | 0.04796 |
| | | TCGA | 164 | 23 | 0.558 | 0.351 | 0.887 | 0.01372 |
| IGG_LUM | CD79A_DNAJC12 | METABRIC | 236 | 147 | 0.847 | 0.724 | 0.992 | 0.03926 |
| | | SCANB | 378 | 46 | 0.751 | 0.569 | 0.993 | 0.04412 |
| | | TCGA | 164 | 23 | 0.573 | 0.372 | 0.883 | 0.01153 |
| IGG_LUM | IGJ_DNAJC12 | SCANB | 378 | 46 | 0.74 | 0.564 | 0.971 | 0.0297 |
| | | TCGA | 164 | 23 | 0.61 | 0.401 | 0.926 | 0.02045 |
| IGG_LUM | POU2AF1_DNAJC12 | METABRIC | 236 | 147 | 0.853 | 0.728 | 1 | 0.04981 |
| | | SCANB | 378 | 46 | 0.741 | 0.56 | 0.98 | 0.03588 |
| IGG_LUM | TNFRSF17_DNAJC12 | SCANB | 378 | 46 | 0.717 | 0.541 | 0.949 | 0.0199 |
| | | TCGA | 164 | 23 | 0.628 | 0.4 | 0.986 | 0.04318 |
| IGG_LUM | CD79A_ESR1 | METABRIC | 236 | 147 | 0.843 | 0.722 | 0.985 | 0.03135 |
| | | TCGA | 164 | 23 | 0.534 | 0.317 | 0.9 | 0.01848 |
| IGG_LUM | CD27_ASPM | SCANB | 378 | 46 | 0.593 | 0.449 | 0.785 | 0.00025 |
| | | SHORTHER_DMFS | 434 | 63 | 0.78 | 0.612 | 0.995 | 0.0458 |
| | | TCGA | 164 | 23 | 0.4 | 0.24 | 0.668 | 0.00046 |
| IGG_PROLIF | CD79A_ASPM | METABRIC | 236 | 147 | 0.833 | 0.715 | 0.97 | 0.01903 |
| | | SCANB | 378 | 46 | 0.598 | 0.456 | 0.785 | 0.00021 |
| | | SHORTHER_DMFS | 434 | 63 | 0.702 | 0.546 | 0.903 | 0.00583 |
| | | SHORTHER_OS | 434 | 87 | 0.789 | 0.638 | 0.975 | 0.0283 |
| | | TCGA | 164 | 23 | 0.41 | 0.266 | 0.633 | 0.00006 |
| IGG_PROLIF | IGJ_ASPM | SCANB | 378 | 46 | 0.61 | 0.469 | 0.793 | 0.00022 |
| | | SHORTHER_DMFS | 434 | 63 | 0.784 | 0.618 | 0.995 | 0.04582 |
| | | TCGA | 164 | 23 | 0.46 | 0.302 | 0.701 | 0.0003 |
| IGG_PROLIF | IL2RG_ASPM | SCANB | 378 | 46 | 0.627 | 0.479 | 0.82 | 0.00065 |
| | | TCGA | 164 | 23 | 0.453 | 0.278 | 0.738 | 0.0015 |
| IGG_PROLIF | LAX1_ASPM | SCANB | 378 | 46 | 0.535 | 0.397 | 0.721 | 0.00004 |
| | | TCGA | 164 | 23 | 0.495 | 0.311 | 0.787 | 0.003 |
| IGG_PROLIF | PIM2_ASPM | METABRIC | 236 | 147 | 0.83 | 0.709 | 0.972 | 0.02048 |
| | | SCANB | 378 | 46 | 0.554 | 0.408 | 0.752 | 0.00015 |
| | | SHORTHER_DMFS | 434 | 63 | 0.779 | 0.606 | 1 | 0.04998 |
| | | TCGA | 164 | 23 | 0.448 | 0.275 | 0.73 | 0.00127 |
| IGG_PROLIF | POU2AF1_ASPM | METABRIC | 236 | 147 | 0.834 | 0.712 | 0.976 | 0.02366 |
| | | SCANB | 378 | 46 | 0.567 | 0.429 | 0.751 | 0.00007 |
| | | SHORTHER_DMFS | 434 | 63 | 0.762 | 0.6 | 0.967 | 0.0257 |
| | | TCGA | 164 | 23 | 0.51 | 0.337 | 0.772 | 0.00143 |
| IGG_PROLIF | TNFRSF17_ASPM | METABRIC | 236 | 147 | 0.855 | 0.732 | 0.999 | 0.04882 |
| | | SCANB | 378 | 46 | 0.555 | 0.418 | 0.737 | 0.00005 |
| | | SHORTHER_DMFS | 434 | 63 | 0.775 | 0.609 | 0.986 | 0.03818 |
| | | TCGA | 164 | 23 | 0.438 | 0.274 | 0.698 | 0.00053 |
| IGG_PROLIF | CD27_EXO1 | METABRIC | 236 | 147 | 0.856 | 0.733 | 0.998 | 0.04729 |
| | | SCANB | 378 | 46 | 0.564 | 0.423 | 0.753 | 0.0001 |
| | | SHORTHER_DMFS | 434 | 63 | 0.695 | 0.54 | 0.895 | 0.0048 |
| | | SHORTHER_OS | 434 | 87 | 0.79 | 0.64 | 0.976 | 0.02872 |
| | | TCGA | 164 | 23 | 0.315 | 0.184 | 0.539 | 0.00003 |
| IGG_PROLIF | CD79A_EXO1 | METABRIC | 236 | 147 | 0.815 | 0.697 | 0.952 | 0.00994 |
| | | SCANB | 378 | 46 | 0.573 | 0.435 | 0.756 | 0.00008 |
| | | SHORTHER_DMFS | 434 | 63 | 0.666 | 0.518 | 0.856 | 0.00149 |
| | | SHORTHER_OS | 434 | 87 | 0.759 | 0.614 | 0.939 | 0.01108 |
| | | TCGA | 164 | 23 | 0.423 | 0.291 | 0.613 | 0.00001 |
| IGG_PROLIF | HLA.C_EXO1 | SCANB | 378 | 46 | 0.734 | 0.557 | 0.967 | 0.02815 |
| | | TCGA | 164 | 23 | 0.541 | 0.339 | 0.865 | 0.01021 |
| IGG_PROLIF | IGJ_EXO1 | SCANB | 378 | 46 | 0.58 | 0.442 | 0.761 | 0.00008 |
| | | SHORTHER_DMFS | 434 | 63 | 0.753 | 0.594 | 0.955 | 0.01933 |
| | | SHORTHER_OS | 434 | 87 | 0.819 | 0.67 | 1 | 0.0496 |
| | | TCGA | 164 | 23 | 0.423 | 0.279 | 0.641 | 0.00005 |
| | | SHORTHER_DMFS | 434 | 63 | 0.72 | 0.557 | 0.931 | 0.01232 |
| | | SHORTHER_OS | 434 | 87 | 0.798 | 0.645 | 0.988 | 0.03819 |
| IGG_PROLIF | IL2RG_EXO1 | METABRIC | 236 | 147 | 0.841 | 0.716 | 0.988 | 0.03497 |
| | | SCANB | 378 | 46 | 0.597 | 0.453 | 0.788 | 0.00027 |
| | | SHORTHER_DMFS | 434 | 63 | 0.745 | 0.581 | 0.955 | 0.0203 |
| | | TCGA | 164 | 23 | 0.352 | 0.211 | 0.587 | 0.00006 |
| IGG_PROLIF | LAX1_EXO1 | SCANB | 378 | 46 | 0.519 | 0.387 | 0.698 | 0.00001 |
| | | SHORTHER_DMFS | 434 | 63 | 0.722 | 0.567 | 0.92 | 0.00851 |
| | | TCGA | 164 | 23 | 0.42 | 0.26 | 0.678 | 0.00039 |
| IGG_PROLIF | PIM2_EXO1 | METABRIC | 236 | 147 | 0.811 | 0.692 | 0.95 | 0.00931 |
| | | SCANB | 378 | 46 | 0.52 | 0.38 | 0.712 | 0.00004 |
| | | SHORTHER_DMFS | 434 | 63 | 0.729 | 0.571 | 0.93 | 0.01109 |
| | | SHORTHER_OS | 434 | 87 | 0.788 | 0.637 | 0.976 | 0.02904 |
| | | TCGA | 164 | 23 | 0.327 | 0.188 | 0.567 | 0.00007 |
| IGG_PROLIF | POU2AF1_EXO1 | METABRIC | 236 | 147 | 0.813 | 0.692 | 0.955 | 0.01155 |
| | | SCANB | 378 | 46 | 0.542 | 0.408 | 0.721 | 0.00003 |
| | | SHORTHER_DMFS | 434 | 63 | 0.691 | 0.54 | 0.884 | 0.00322 |
| | | SHORTHER_OS | 434 | 87 | 0.778 | 0.631 | 0.958 | 0.01838 |
| | | TCGA | 164 | 23 | 0.485 | 0.331 | 0.711 | 0.00021 |
| IGG_PROLIF | TNFRSF17_EXO1 | METABRIC | 236 | 147 | 0.835 | 0.715 | 0.975 | 0.02271 |
| | | SCANB | 378 | 46 | 0.521 | 0.389 | 0.7 | 0.00001 |
| | | SHORTHER_DMFS | 434 | 63 | 0.691 | 0.54 | 0.884 | 0.00333 |
| | | SHORTHER_OS | 434 | 87 | 0.801 | 0.649 | 0.987 | 0.0377 |
| | | TCGA | 164 | 23 | 0.388 | 0.242 | 0.622 | 0.00008 |
| IGG_PROLIF | CD27_KIF23 | METABRIC | 236 | 147 | 0.839 | 0.715 | 0.983 | 0.02967 |
| | | SCANB | 378 | 46 | 0.607 | 0.458 | 0.804 | 0.0005 |
| | | SHORTHER_DMFS | 434 | 63 | 0.706 | 0.548 | 0.91 | 0.00729 |
| | | SHORTHER_OS | 434 | 87 | 0.792 | 0.638 | 0.983 | 0.03412 |
| | | TCGA | 164 | 23 | 0.359 | 0.21 | 0.612 | 0.00017 |
| IGG_PROLIF | CD79A_KIF23 | METABRIC | 236 | 147 | 0.798 | 0.679 | 0.939 | 0.00634 |
| | | SCANB | 378 | 46 | 0.604 | 0.458 | 0.797 | 0.00036 |
| | | SHORTHER_DMFS | 434 | 63 | 0.659 | 0.507 | 0.856 | 0.00178 |
| | | SHORTHER_OS | 434 | 87 | 0.752 | 0.603 | 0.937 | 0.01124 |
| | | TCGA | 164 | 23 | 0.402 | 0.264 | 0.613 | 0.00002 |
| IGG_PROLIF | IGJ_KIF23 | SCANB | 378 | 46 | 0.608 | 0.465 | 0.796 | 0.00029 |
| | | SHORTHER_DMFS | 434 | 63 | 0.754 | 0.593 | 0.958 | 0.02083 |
| | | SHORTHER_OS | 434 | 87 | 0.818 | 0.669 | 1 | 0.04969 |
| | | TCGA | 164 | 23 | 0.452 | 0.296 | 0.689 | 0.00023 |
| | | SHORTHER_DMFS | 434 | 63 | 0.721 | 0.554 | 0.939 | 0.01511 |
| | | SHORTHER_OS | 434 | 87 | 0.797 | 0.641 | 0.992 | 0.04216 |
| IGG_PROLIF | IL2RG_KIF23 | METABRIC | 236 | 147 | 0.828 | 0.699 | 0.979 | 0.02744 |
| | | SCANB | 378 | 46 | 0.644 | 0.492 | 0.841 | 0.00126 |
| | | SHORTHER_DMFS | 434 | 63 | 0.744 | 0.576 | 0.959 | 0.02258 |
| | | TCGA | 164 | 23 | 0.402 | 0.238 | 0.678 | 0.00063 |
| IGG_PROLIF | LAX1_KIF23 | SCANB | 378 | 46 | 0.555 | 0.414 | 0.745 | 0.00009 |
| | | SHORTHER_DMFS | 434 | 63 | 0.741 | 0.577 | 0.951 | 0.01846 |
| | | TCGA | 164 | 23 | 0.489 | 0.308 | 0.775 | 0.00231 |
| IGG_PROLIF | PIM2_KIF23 | METABRIC | 236 | 147 | 0.786 | 0.668 | 0.925 | 0.00367 |
| | | SCANB | 378 | 46 | 0.57 | 0.42 | 0.773 | 0.00031 |
| | | SHORTHER_DMFS | 434 | 63 | 0.719 | 0.553 | 0.936 | 0.01402 |
| | | SHORTHER_OS | 434 | 87 | 0.784 | 0.627 | 0.98 | 0.03237 |
| | | TCGA | 164 | 23 | 0.335 | 0.183 | 0.614 | 0.0004 |
| IGG_PROLIF | POU2AF1_KIF23 | METABRIC | 236 | 147 | 0.795 | 0.674 | 0.939 | 0.00674 |
| | | SCANB | 378 | 46 | 0.573 | 0.432 | 0.762 | 0.00013 |
| | | SHORTHER_DMFS | 434 | 63 | 0.705 | 0.55 | 0.903 | 0.00569 |
| | | SHORTHER_OS | 434 | 87 | 0.783 | 0.633 | 0.969 | 0.02473 |
| | | TCGA | 164 | 23 | 0.495 | 0.326 | 0.751 | 0.00094 |
| IGG_PROLIF | TNFRSF17_KIF23 | METABRIC | 236 | 147 | 0.809 | 0.69 | 0.949 | 0.00935 |
| | | SCANB | 378 | 46 | 0.553 | 0.413 | 0.739 | 0.00006 |
| | | SHORTHER_DMFS | 434 | 63 | 0.703 | 0.544 | 0.908 | 0.00687 |
| | | TCGA | 164 | 23 | 0.452 | 0.293 | 0.698 | 0.00034 |
| IGG_PROLIF | CD27_NEK2 | SCANB | 378 | 46 | 0.625 | 0.473 | 0.826 | 0.00095 |
| | | TCGA | 164 | 23 | 0.386 | 0.233 | 0.637 | 0.0002 |
| IGG_PROLIF | CD79A_NEK2 | SCANB | 378 | 46 | 0.619 | 0.471 | 0.814 | 0.00059 |
| | | SHORTHER_DMFS | 434 | 63 | 0.717 | 0.557 | 0.924 | 0.01004 |
| | | TCGA | 164 | 23 | 0.42 | 0.279 | 0.634 | 0.00004 |
| IGG_PROLIF | IGJ_NEK2 | SCANB | 378 | 46 | 0.621 | 0.474 | 0.814 | 0.00055 |
| | | TCGA | 164 | 23 | 0.457 | 0.301 | 0.693 | 0.00023 |
| IGG_PROLIF | IL2RG_NEK2 | SCANB | 378 | 46 | 0.66 | 0.504 | 0.865 | 0.00258 |
| | | TCGA | 164 | 23 | 0.437 | 0.271 | 0.704 | 0.00068 |
| IGG_PROLIF | LAX1_NEK2 | SCANB | 378 | 46 | 0.572 | 0.427 | 0.768 | 0.0002 |
| | | TCGA | 164 | 23 | 0.493 | 0.309 | 0.787 | 0.00305 |
| IGG_PROLIF | PIM2_NEK2 | SCANB | 378 | 46 | 0.585 | 0.43 | 0.798 | 0.0007 |
| | | TCGA | 164 | 23 | 0.41 | 0.248 | 0.679 | 0.00053 |
| IGG_PROLIF | POU2AF1_NEK2 | SCANB | 378 | 46 | 0.591 | 0.446 | 0.784 | 0.00026 |
| | | SHORTHER_DMFS | 434 | 63 | 0.771 | 0.606 | 0.981 | 0.03424 |
| | | TCGA | 164 | 23 | 0.512 | 0.34 | 0.77 | 0.00132 |
| IGG_PROLIF | TNFRSF17_NEK2 | SCANB | 378 | 46 | 0.573 | 0.43 | 0.762 | 0.00013 |
| | | SHORTHER_DMFS | 434 | 63 | 0.779 | 0.612 | 0.992 | 0.04325 |
| | | TCGA | 164 | 23 | 0.439 | 0.278 | 0.693 | 0.00042 |
| LUM_PROLIF | BCL2_EXO1 | SCANB | 378 | 46 | 0.675 | 0.499 | 0.911 | 0.01034 |
| | | TCGA | 164 | 23 | 0.599 | 0.381 | 0.943 | 0.02672 |
| LUM_PROLIF | BCL2_KIF23 | METABRIC | 236 | 147 | 0.848 | 0.724 | 0.992 | 0.03902 |
| | | SCANB | 378 | 46 | 0.692 | 0.519 | 0.923 | 0.01218 |
| LUM_PROLIF | BCL2_NEK2 | SCANB | 378 | 46 | 0.698 | 0.518 | 0.94 | 0.01776 |
| | | TCGA | 164 | 23 | 0.633 | 0.402 | 0.998 | 0.04918 |

TABLE 7B
List of 78 combination scores significantly associated with poor survival outcome in 2 or more datasets

| | | | | | | 95% CI | 95% CI | |
|---|---|---|---|---|---|---|---|---|
| | | | | n | Hazard | lower | higher | |
| Gene combination | Signatures | Dataset | n | events | Ratio | limit | limit | p-value |
| CD27_CD79A | IGG_IGG | SHORTHER_OS | 434 | 87 | 1.289 | 1.038 | 1.601 | 0.02148 |
| | | SCANB | 378 | 46 | 1.388 | 1.064 | 1.809 | 0.01548 |
| | | SHORTHER_DMFS | 434 | 63 | 1.404 | 1.1 | 1.792 | 0.00635 |
| | | TCGA | 164 | 23 | 1.684 | 1.195 | 2.373 | 0.0029 |
| CXCL8_CD27 | IGG_IGG | SCANB | 378 | 46 | 1.419 | 1.073 | 1.877 | 0.01399 |
| | | TCGA | 164 | 23 | 1.587 | 1.066 | 2.363 | 0.0229 |
| CXCL8_CD79A | IGG_IGG | SHORTHER_OS | 434 | 87 | 1.253 | 1.007 | 1.559 | 0.04298 |
| | | SHORTHER_DMFS | 434 | 63 | 1.33 | 1.033 | 1.711 | 0.02688 |
| | | SCANB | 378 | 46 | 1.525 | 1.152 | 2.018 | 0.00318 |
| | | TCGA | 164 | 23 | 1.744 | 1.206 | 2.52 | 0.00309 |
| CXCL8_IGJ | IGG_IGG | SCANB | 378 | 46 | 1.506 | 1.156 | 1.962 | 0.00238 |
| | | TCGA | 164 | 23 | 1.702 | 1.145 | 2.529 | 0.00852 |
| CXCL8_POU2AF1 | IGG_IGG | TCGA | 164 | 23 | 1.5 | 1.036 | 2.172 | 0.03173 |
| | | SCANB | 378 | 46 | 1.555 | 1.179 | 2.052 | 0.00179 |
| CXCL8_TNFRSF17 | IGG_IGG | SCANB | 378 | 46 | 1.606 | 1.212 | 2.128 | 0.00096 |
| | | TCGA | 164 | 23 | 1.618 | 1.107 | 2.365 | 0.01287 |
| HLA.C_CD27 | IGG_IGG | SCANB | 378 | 46 | 1.614 | 1.239 | 2.103 | 0.00039 |
| | | TCGA | 164 | 23 | 2.439 | 1.509 | 3.942 | 0.00027 |
| HLA.C_CD79A | IGG_IGG | SHORTHER_DMFS | 434 | 63 | 1.36 | 1.074 | 1.721 | 0.01059 |
| | | SCANB | 378 | 46 | 1.612 | 1.233 | 2.109 | 0.00049 |
| | | TCGA | 164 | 23 | 2.354 | 1.54 | 3.599 | 0.00008 |
| HLA.C_IGJ | IGG_IGG | SCANB | 378 | 46 | 1.547 | 1.198 | 1.998 | 0.00084 |
| | | TCGA | 164 | 23 | 2.08 | 1.342 | 3.224 | 0.00106 |
| HLA.C_IL2RG | IGG_IGG | SCANB | 378 | 46 | 1.548 | 1.19 | 2.015 | 0.00115 |
| | | TCGA | 164 | 23 | 2.04 | 1.253 | 3.323 | 0.00417 |
| HLA.C_PIM2 | IGG_IGG | TCGA | 164 | 23 | 1.654 | 1.053 | 2.597 | 0.02882 |
| | | SCANB | 378 | 46 | 1.676 | 1.23 | 2.282 | 0.00105 |
| HLA.C_POU2AF1 | IGG_IGG | SCANB | 378 | 46 | 1.694 | 1.285 | 2.233 | 0.00019 |
| | | TCGA | 164 | 23 | 1.781 | 1.165 | 2.723 | 0.00767 |
| HLA.C_TNFRSF17 | IGG_IGG | SCANB | 378 | 46 | 1.726 | 1.31 | 2.273 | 0.0001 |
| | | TCGA | 164 | 23 | 1.983 | 1.287 | 3.056 | 0.00192 |
| IGLV3.25_IGL | IGG_IGG | SHORTHER_OS | 434 | 87 | 1.245 | 1.022 | 1.517 | 0.02967 |
| | | SHORTHER_DMFS | 434 | 63 | 1.398 | 1.118 | 1.747 | 0.00325 |
| IL2RG_CD79A | IGG_IGG | SHORTHER_OS | 434 | 87 | 1.325 | 1.056 | 1.664 | 0.01508 |
| | | SCANB | 378 | 46 | 1.439 | 1.099 | 1.885 | 0.00823 |
| | | SHORTHER_DMFS | 434 | 63 | 1.442 | 1.111 | 1.87 | 0.00588 |
| | | TCGA | 164 | 23 | 1.906 | 1.328 | 2.735 | 0.00047 |
| IL2RG_IGJ | IGG_IGG | SCANB | 378 | 46 | 1.338 | 1.011 | 1.77 | 0.0414 |
| | | TCGA | 164 | 23 | 1.691 | 1.101 | 2.598 | 0.01638 |
| IL2RG_TNFRSF17 | IGG_IGG | SCANB | 378 | 46 | 1.488 | 1.125 | 1.967 | 0.0053 |
| | | TCGA | 164 | 23 | 1.585 | 1.045 | 2.404 | 0.03039 |
| LAX1_CD79A | IGG_IGG | SHORTHER_OS | 434 | 87 | 1.302 | 1.07 | 1.586 | 0.00853 |
| | | SHORTHER_DMFS | 434 | 63 | 1.364 | 1.085 | 1.716 | 0.00786 |
| | | TCGA | 164 | 23 | 1.858 | 1.323 | 2.611 | 0.00036 |
| LAX1_POU2AF1 | IGG_IGG | SHORTHER_DMFS | 434 | 63 | 1.296 | 1.02 | 1.647 | 0.03362 |
| | | SHORTHER_OS | 434 | 87 | 1.319 | 1.074 | 1.62 | 0.00817 |
| LAX1_TNFRSF17 | IGG_IGG | SCANB | 378 | 46 | 1.311 | 1.003 | 1.715 | 0.04779 |
| | | TCGA | 164 | 23 | 1.769 | 1.144 | 2.737 | 0.01038 |
| NTN3_CD27 | IGG_IGG | SCANB | 378 | 46 | 1.395 | 1.054 | 1.847 | 0.01992 |
| | | TCGA | 164 | 23 | 1.961 | 1.216 | 3.16 | 0.00571 |
| NTN3_CD79A | IGG_IGG | METABRIC | 236 | 147 | 1.221 | 1.032 | 1.445 | 0.02015 |
| | | SHORTHER_OS | 434 | 87 | 1.278 | 1.026 | 1.592 | 0.02862 |
| | | SHORTHER_DMFS | 434 | 63 | 1.341 | 1.036 | 1.734 | 0.02556 |
| | | SCANB | 378 | 46 | 1.474 | 1.119 | 1.941 | 0.00574 |
| | | TCGA | 164 | 23 | 2.103 | 1.374 | 3.217 | 0.00061 |
| NTN3_IGJ | IGG_IGG | SCANB | 378 | 46 | 1.459 | 1.115 | 1.91 | 0.00587 |
| | | TCGA | 164 | 23 | 1.883 | 1.269 | 2.794 | 0.00167 |
| NTN3_IL2RG | IGG_IGG | SCANB | 378 | 46 | 1.344 | 1.01 | 1.788 | 0.04235 |
| | | TCGA | 164 | 23 | 1.666 | 1.043 | 2.661 | 0.03276 |
| NTN3_PIM2 | IGG_IGG | METABRIC | 236 | 147 | 1.205 | 1.015 | 1.43 | 0.03343 |
| | | SCANB | 378 | 46 | 1.347 | 1.013 | 1.791 | 0.04032 |
| NTN3_POU2AF1 | IGG_IGG | METABRIC | 236 | 147 | 1.215 | 1.026 | 1.439 | 0.02429 |
| | | SHORTHER_OS | 434 | 87 | 1.25 | 1.001 | 1.561 | 0.04933 |
| | | SCANB | 378 | 46 | 1.503 | 1.14 | 1.982 | 0.00384 |
| | | TCGA | 164 | 23 | 1.653 | 1.107 | 2.47 | 0.0141 |
| NTN3_TNFRSF17 | IGG_IGG | METABRIC | 236 | 147 | 1.187 | 1.002 | 1.408 | 0.0479 |
| | | SCANB | 378 | 46 | 1.544 | 1.172 | 2.035 | 0.00203 |
| | | TCGA | 164 | 23 | 1.88 | 1.208 | 2.927 | 0.00515 |
| PIM2_CD79A | IGG_IGG | SHORTHER_OS | 434 | 87 | 1.217 | 1.002 | 1.478 | 0.04802 |
| | | SHORTHER_DMFS | 434 | 63 | 1.389 | 1.105 | 1.746 | 0.00493 |
| | | SCANB | 378 | 46 | 1.397 | 1.086 | 1.798 | 0.00938 |
| | | TCGA | 164 | 23 | 1.986 | 1.392 | 2.834 | 0.00016 |
| PIM2_IGJ | IGG_IGG | SCANB | 378 | 46 | 1.362 | 1.045 | 1.775 | 0.02213 |
| | | TCGA | 164 | 23 | 1.724 | 1.179 | 2.521 | 0.00494 |
| PIM2_POU2AF1 | IGG_IGG | SCANB | 378 | 46 | 1.463 | 1.143 | 1.873 | 0.00256 |
| | | TCGA | 164 | 23 | 1.481 | 1.03 | 2.131 | 0.03418 |
| PIM2_TNFRSF17 | IGG_IGG | SCANB | 378 | 46 | 1.518 | 1.181 | 1.951 | 0.00112 |
| | | TCGA | 164 | 23 | 1.675 | 1.147 | 2.448 | 0.00762 |
| POU2AF1_CD79A | IGG_IGG | SHORTHER_DMFS | 434 | 63 | 1.271 | 1.012 | 1.597 | 0.03955 |
| | | TCGA | 164 | 23 | 1.661 | 1.264 | 2.182 | 0.00027 |
| AGR3_CD79A | LUM_IGG | METABRIC | 236 | 147 | 1.223 | 1.043 | 1.435 | 0.01307 |
| | | SHORTHER_DMFS | 434 | 63 | 1.296 | 1.004 | 1.673 | 0.04676 |
| BCL2_CD79A | LUM_IGG | SHORTHER_DMFS | 434 | 63 | 1.274 | 1.002 | 1.62 | 0.04796 |
| | | TCGA | 164 | 23 | 1.793 | 1.127 | 2.852 | 0.01372 |
| DNAJC12_CD79A | LUM_IGG | METABRIC | 236 | 147 | 1.18 | 1.008 | 1.382 | 0.03926 |
| | | SCANB | 378 | 46 | 1.331 | 1.008 | 1.758 | 0.04412 |
| | | TCGA | 164 | 23 | 1.746 | 1.133 | 2.691 | 0.01153 |
| DNAJC12_IGJ | LUM_IGG | SCANB | 378 | 46 | 1.352 | 1.03 | 1.775 | 0.0297 |
| | | TCGA | 164 | 23 | 1.64 | 1.079 | 2.492 | 0.02045 |
| DNAJC12_POU2AF1 | LUM_IGG | METABRIC | 236 | 147 | 1.172 | 1 | 1.374 | 0.04981 |
| | | SCANB | 378 | 46 | 1.349 | 1.02 | 1.785 | 0.03588 |
| DNAJC12_TNFRSF17 | LUM_IGG | SCANB | 378 | 46 | 1.395 | 1.054 | 1.847 | 0.0199 |
| | | TCGA | 164 | 23 | 1.592 | 1.014 | 2.498 | 0.04318 |
| ESR1_CD79A | LUM_IGG | METABRIC | 236 | 147 | 1.186 | 1.015 | 1.386 | 0.03135 |
| | | TCGA | 164 | 23 | 1.872 | 1.111 | 3.155 | 0.01848 |
| AGR3_HLA.C | LUM_IGG | METABRIC | 236 | 147 | 1.187 | 1.002 | 1.407 | 0.04755 |
| | | SCANB | 378 | 46 | 1.341 | 1.024 | 1.756 | 0.03305 |
| ASPM_CD27 | PROLIF_IGG | SHORTHER_DMFS | 434 | 63 | 1.281 | 1.005 | 1.634 | 0.0458 |
| | | SCANB | 378 | 46 | 1.685 | 1.274 | 2.229 | 0.00025 |
| | | TCGA | 164 | 23 | 2.499 | 1.497 | 4.172 | 0.00046 |
| ASPM_CD79A | PROLIF_IGG | METABRIC | 236 | 147 | 1.201 | 1.03 | 1.399 | 0.01903 |
| | | SHORTHER_OS | 434 | 87 | 1.268 | 1.026 | 1.567 | 0.0283 |
| | | SHORTHER_DMFS | 434 | 63 | 1.424 | 1.108 | 1.831 | 0.00583 |
| | | SCANB | 378 | 46 | 1.672 | 1.274 | 2.194 | 0.00021 |
| | | TCGA | 164 | 23 | 2.439 | 1.581 | 3.762 | 0.00006 |
| ASPM_IGJ | PROLIF_IGG | SHORTHER_DMFS | 434 | 63 | 1.275 | 1.005 | 1.619 | 0.04582 |
| | | SCANB | 378 | 46 | 1.641 | 1.262 | 2.133 | 0.00022 |
| | | TCGA | 164 | 23 | 2.172 | 1.427 | 3.306 | 0.0003 |
| ASPM_IL2RG | PROLIF_IGG | SCANB | 378 | 46 | 1.596 | 1.22 | 2.088 | 0.00065 |
| | | TCGA | 164 | 23 | 2.208 | 1.354 | 3.6 | 0.0015 |
| ASPM_LAX1 | PROLIF_IGG | SCANB | 378 | 46 | 1.869 | 1.388 | 2.517 | 0.00004 |
| | | TCGA | 164 | 23 | 2.021 | 1.27 | 3.216 | 0.003 |
| ASPM_PIM2 | PROLIF_IGG | METABRIC | 236 | 147 | 1.205 | 1.029 | 1.41 | 0.02048 |
| | | SHORTHER_DMFS | 434 | 63 | 1.284 | 1 | 1.649 | 0.04998 |
| | | SCANB | 378 | 46 | 1.806 | 1.33 | 2.451 | 0.00015 |
| | | TCGA | 164 | 23 | 2.233 | 1.37 | 3.639 | 0.00127 |
| ASPM_POU2AF1 | PROLIF_IGG | METABRIC | 236 | 147 | 1.199 | 1.025 | 1.404 | 0.02366 |
| | | SHORTHER_DMFS | 434 | 63 | 1.313 | 1.034 | 1.668 | 0.0257 |
| | | SCANB | 378 | 46 | 1.763 | 1.332 | 2.334 | 0.00007 |
| | | TCGA | 164 | 23 | 1.96 | 1.296 | 2.963 | 0.00143 |
| ASPM_TNFRSF17 | PROLIF_IGG | METABRIC | 236 | 147 | 1.169 | 1.001 | 1.366 | 0.04882 |
| | | SHORTHER_DMFS | 434 | 63 | 1.291 | 1.014 | 1.643 | 0.03818 |
| | | SCANB | 378 | 46 | 1.802 | 1.357 | 2.393 | 0.00005 |
| | | TCGA | 164 | 23 | 2.285 | 1.432 | 3.645 | 0.00053 |
| EXO1_CD27 | PROLIF_IGG | METABRIC | 236 | 147 | 1.169 | 1.002 | 1.364 | 0.04729 |
| | | SHORTHER_OS | 434 | 87 | 1.265 | 1.025 | 1.562 | 0.02872 |
| | | SHORTHER_DMFS | 434 | 63 | 1.438 | 1.117 | 1.851 | 0.0048 |
| | | SCANB | 378 | 46 | 1.773 | 1.328 | 2.366 | 0.0001 |
| | | TCGA | 164 | 23 | 3.176 | 1.855 | 5.437 | 0.00003 |
| EXO1_CD79A | PROLIF_IGG | METABRIC | 236 | 147 | 1.228 | 1.05 | 1.435 | 0.00994 |
| | | SHORTHER_OS | 434 | 87 | 1.317 | 1.065 | 1.629 | 0.01108 |
| | | SHORTHER_DMFS | 434 | 63 | 1.502 | 1.168 | 1.93 | 0.00149 |
| | | SCANB | 378 | 46 | 1.745 | 1.324 | 2.3 | 0.00008 |
| | | TCGA | 164 | 23 | 2.366 | 1.63 | 3.434 | 0.00001 |
| EXO1_HLA.C | PROLIF_IGG | SCANB | 378 | 46 | 1.363 | 1.034 | 1.797 | 0.02815 |
| | | TCGA | 164 | 23 | 1.848 | 1.157 | 2.953 | 0.01021 |
| EXO1_IGJ | PROLIF_IGG | SHORTHER_OS | 434 | 87 | 1.221 | 1 | 1.492 | 0.0496 |
| | | SHORTHER_DMFS | 434 | 63 | 1.327 | 1.047 | 1.683 | 0.01933 |
| | | SCANB | 378 | 46 | 1.724 | 1.314 | 2.262 | 0.00008 |
| | | TCGA | 164 | 23 | 2.366 | 1.561 | 3.586 | 0.00005 |
| EXO1_IGL | PROLIF_IGG | SHORTHER_OS | 434 | 87 | 1.253 | 1.012 | 1.551 | 0.03819 |
| | | SHORTHER_DMFS | 434 | 63 | 1.389 | 1.074 | 1.796 | 0.01232 |
| EXO1_IL2RG | PROLIF_IGG | METABRIC | 236 | 147 | 1.189 | 1.012 | 1.397 | 0.03497 |
| | | SHORTHER_DMFS | 434 | 63 | 1.343 | 1.047 | 1.722 | 0.0203 |
| | | SCANB | 378 | 46 | 1.674 | 1.268 | 2.209 | 0.00027 |
| | | TCGA | 164 | 23 | 2.84 | 1.704 | 4.735 | 0.00006 |
| EXO1_LAX1 | PROLIF_IGG | SHORTHER_DMFS | 434 | 63 | 1.385 | 1.087 | 1.765 | 0.00851 |
| | | SCANB | 378 | 46 | 1.925 | 1.433 | 2.587 | 0.00001 |
| | | TCGA | 164 | 23 | 2.384 | 1.476 | 3.851 | 0.00039 |
| EXO1_PIM2 | PROLIF_IGG | METABRIC | 236 | 147 | 1.234 | 1.053 | 1.445 | 0.00931 |
| | | SHORTHER_OS | 434 | 87 | 1.269 | 1.025 | 1.571 | 0.02904 |
| | | SHORTHER_DMFS | 434 | 63 | 1.372 | 1.075 | 1.75 | 0.01109 |
| | | SCANB | 378 | 46 | 1.922 | 1.405 | 2.629 | 0.00004 |
| | | TCGA | 164 | 23 | 3.062 | 1.764 | 5.316 | 0.00007 |
| EXO1_POU2AF1 | PROLIF_IGG | METABRIC | 236 | 147 | 1.231 | 1.048 | 1.446 | 0.01155 |
| | | SHORTHER_OS | 434 | 87 | 1.286 | 1.043 | 1.585 | 0.01838 |
| | | SHORTHER_DMFS | 434 | 63 | 1.447 | 1.132 | 1.851 | 0.00322 |
| | | SCANB | 378 | 46 | 1.845 | 1.387 | 2.453 | 0.00003 |
| | | TCGA | 164 | 23 | 2.063 | 1.407 | 3.024 | 0.00021 |
| EXO1_TNFRSF17 | PROLIF_IGG | METABRIC | 236 | 147 | 1.198 | 1.026 | 1.399 | 0.02271 |
| | | SHORTHER_OS | 434 | 87 | 1.249 | 1.013 | 1.541 | 0.0377 |
| | | SHORTHER_DMFS | 434 | 63 | 1.447 | 1.131 | 1.851 | 0.00333 |
| | | SCANB | 378 | 46 | 1.918 | 1.429 | 2.574 | 0.00001 |
| | | TCGA | 164 | 23 | 2.575 | 1.608 | 4.124 | 0.00008 |
| KIF23_CD27 | PROLIF_IGG | METABRIC | 236 | 147 | 1.193 | 1.018 | 1.398 | 0.02967 |
| | | SHORTHER_OS | 434 | 87 | 1.263 | 1.018 | 1.568 | 0.03412 |
| | | SHORTHER_DMFS | 434 | 63 | 1.416 | 1.098 | 1.826 | 0.00729 |
| | | SCANB | 378 | 46 | 1.647 | 1.243 | 2.182 | 0.0005 |
| | | TCGA | 164 | 23 | 2.786 | 1.634 | 4.752 | 0.00017 |
| KIF23_CD79A | PROLIF_IGG | METABRIC | 236 | 147 | 1.252 | 1.066 | 1.472 | 0.00634 |
| | | SHORTHER_OS | 434 | 87 | 1.33 | 1.067 | 1.657 | 0.01124 |
| | | SHORTHER_DMFS | 434 | 63 | 1.519 | 1.169 | 1.974 | 0.00178 |
| | | SCANB | 378 | 46 | 1.655 | 1.255 | 2.183 | 0.00036 |
| | | TCGA | 164 | 23 | 2.486 | 1.632 | 3.787 | 0.00002 |
| KIF23_IGJ | PROLIF_IGG | SHORTHER_OS | 434 | 87 | 1.223 | 1 | 1.495 | 0.04969 |
| | | SHORTHER_DMFS | 434 | 63 | 1.326 | 1.044 | 1.686 | 0.02083 |
| | | SCANB | 378 | 46 | 1.644 | 1.257 | 2.152 | 0.00029 |
| | | TCGA | 164 | 23 | 2.213 | 1.451 | 3.376 | 0.00023 |
| KIF23_IGL | PROLIF_IGG | SHORTHER_OS | 434 | 87 | 1.254 | 1.008 | 1.561 | 0.04216 |
| | | SHORTHER_DMFS | 434 | 63 | 1.387 | 1.065 | 1.805 | 0.01511 |
| KIF23_IL2RG | PROLIF_IGG | METABRIC | 236 | 147 | 1.208 | 1.021 | 1.43 | 0.02744 |
| | | SHORTHER_DMFS | 434 | 63 | 1.345 | 1.043 | 1.735 | 0.02258 |
| | | SCANB | 378 | 46 | 1.554 | 1.189 | 2.031 | 0.00126 |
| | | TCGA | 164 | 23 | 2.487 | 1.475 | 4.194 | 0.00063 |
| KIF23_LAX1 | PROLIF_IGG | SHORTHER_DMFS | 434 | 63 | 1.35 | 1.052 | 1.732 | 0.01846 |
| | | SCANB | 378 | 46 | 1.802 | 1.343 | 2.417 | 0.00009 |
| | | TCGA | 164 | 23 | 2.046 | 1.291 | 3.244 | 0.00231 |
| KIF23_PIM2 | PROLIF_IGG | METABRIC | 236 | 147 | 1.273 | 1.082 | 1.497 | 0.00367 |
| | | SHORTHER_OS | 434 | 87 | 1.276 | 1.021 | 1.596 | 0.03237 |
| | | SHORTHER_DMFS | 434 | 63 | 1.39 | 1.069 | 1.808 | 0.01402 |
| | | SCANB | 378 | 46 | 1.755 | 1.293 | 2.381 | 0.00031 |
| | | TCGA | 164 | 23 | 2.984 | 1.63 | 5.462 | 0.0004 |
| KIF23_POU2AF1 | PROLIF_IGG | METABRIC | 236 | 147 | 1.257 | 1.065 | 1.484 | 0.00674 |
| | | SHORTHER_OS | 434 | 87 | 1.276 | 1.032 | 1.58 | 0.02473 |
| | | SHORTHER_DMFS | 434 | 63 | 1.418 | 1.107 | 1.817 | 0.00569 |
| | | SCANB | 378 | 46 | 1.744 | 1.312 | 2.317 | 0.00013 |
| | | TCGA | 164 | 23 | 2.021 | 1.332 | 3.066 | 0.00094 |
| KIF23_TNFRSF17 | PROLIF_IGG | METABRIC | 236 | 147 | 1.235 | 1.053 | 1.449 | 0.00935 |
| | | SHORTHER_DMFS | 434 | 63 | 1.423 | 1.102 | 1.838 | 0.00687 |
| | | SCANB | 378 | 46 | 1.809 | 1.353 | 2.419 | 0.00006 |
| | | TCGA | 164 | 23 | 2.213 | 1.433 | 3.417 | 0.00034 |
| NEK2_CD27 | PROLIF_IGG | SCANB | 378 | 46 | 1.599 | 1.21 | 2.112 | 0.00095 |
| | | TCGA | 164 | 23 | 2.593 | 1.57 | 4.284 | 0.0002 |
| NEK2_CD79A | PROLIF_IGG | SHORTHER_DMFS | 434 | 63 | 1.394 | 1.083 | 1.796 | 0.01004 |
| | | SCANB | 378 | 46 | 1.616 | 1.229 | 2.124 | 0.00059 |
| | | TCGA | 164 | 23 | 2.378 | 1.578 | 3.585 | 0.00004 |
| NEK2_IGJ | PROLIF_IGG | SCANB | 378 | 46 | 1.61 | 1.229 | 2.11 | 0.00055 |
| | | TCGA | 164 | 23 | 2.19 | 1.442 | 3.325 | 0.00023 |
| NEK2_IL2RG | PROLIF_IGG | SCANB | 378 | 46 | 1.514 | 1.156 | 1.983 | 0.00258 |
| | | TCGA | 164 | 23 | 2.288 | 1.42 | 3.688 | 0.00068 |
| NEK2_LAX1 | PROLIF_IGG | SCANB | 378 | 46 | 1.747 | 1.302 | 2.344 | 0.0002 |
| | | TCGA | 164 | 23 | 2.026 | 1.27 | 3.234 | 0.00305 |
| NEK2_PIM2 | PROLIF_IGG | SCANB | 378 | 46 | 1.708 | 1.253 | 2.328 | 0.0007 |
| | | TCGA | 164 | 23 | 2.437 | 1.473 | 4.033 | 0.00053 |
| NEK2_POU2AF1 | PROLIF_IGG | SHORTHER_DMFS | 434 | 63 | 1.298 | 1.02 | 1.651 | 0.03424 |
| | | SCANB | 378 | 46 | 1.691 | 1.276 | 2.241 | 0.00026 |
| | | TCGA | 164 | 23 | 1.954 | 1.298 | 2.941 | 0.00132 |
| NEK2_TNFRSF17 | PROLIF_IGG | SHORTHER_DMFS | 434 | 63 | 1.283 | 1.008 | 1.634 | 0.04325 |
| | | SCANB | 378 | 46 | 1.746 | 1.312 | 2.325 | 0.00013 |
| | | TCGA | 164 | 23 | 2.28 | 1.442 | 3.604 | 0.00042 |
| EXO1_BCL2 | PROLIF_LUM | SCANB | 378 | 46 | 1.482 | 1.097 | 2.002 | 0.01034 |
| | | TCGA | 164 | 23 | 1.669 | 1.061 | 2.624 | 0.02672 |
| KIF23_BCL2 | PROLIF_LUM | METABRIC | 236 | 147 | 1.18 | 1.008 | 1.38 | 0.03902 |
| | | SCANB | 378 | 46 | 1.446 | 1.084 | 1.928 | 0.01218 |
| NEK2_BCL2 | PROLIF_LUM | SCANB | 378 | 46 | 1.433 | 1.064 | 1.929 | 0.01776 |
| | | TCGA | 164 | 23 | 1.579 | 1.002 | 2.489 | 0.04918 |

The combination scores indicative of good prognosis represent different combinations of the 3 signatures (i.e., immune-proliferation, immune-luminal, luminal-proliferation and immune-immune). Specifically, 45% (n=35) of the combination scores are pairs composed of genes from the immune-proliferation signatures, 10% (n=8) are pairs composed of genes coming from the immune-luminal signatures, and 4% (n=3) are pairs composed of genes from the luminal-proliferation signatures (Table 8). The combination scores indicative of poor prognosis represent different combinations of the 3 signatures (i.e., proliferation-immune, luminal-immune, proliferation-luminal and immune-immune). Specifically, 45% (n=35) of the combination scores are pairs composed of genes from the proliferation-immune signatures, 10% (n=8) are pairs composed of genes coming from the luminal-immune signatures, and 4% (n=3) are pairs composed of genes from the proliferation-luminal signatures (Table 8).

TABLE 8

| | | Gene 2 | | |
|---|---|---|---|---|
| | | IGG | LUM | PROLIF |
| Gene | IGG | 32 | 8 | 35 |
| 1 | LUM | 8 | 0 | 3 |
| | PROLIF | 35 | 3 | 0 |

*IGG: Immune signature, LUM: luminal signature, PROLIF: proliferation signature

### Example 2.7. Combination of 2 Genes Tracking the Luminal, HER2 Amplicon, Proliferation and Immune Signatures is Predictive of Pathological Complete Response (pCR)

The HER2DX pCR score of the HER2DX assay consists of 27 genes [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 and TCAP] and predicts pCR in patients with HER2-positive (HER2+) breast cancer following neoadjuvant systemic anti-HER2-based therapy. The 27 genes are part of one of the following 4 gene expression signatures: Luminal differentiation signature (n=5 genes), HER2 amplicon signature (n=4), tumor cell proliferation signature (n=4) and immune signature (n=14).

We evaluated the association of gene pairs (i.e., combination of 2 genes) included in the 4 gene expression signatures across 3 different datasets of patients with early-stage HER2+ breast cancer treated with neoadjuvant systemic anti-HER2-based therapy, including: 1) Cohort 1, 117 patients with HER2+ breast cancer treated with neoadjuvant anti-HER2-based chemotherapy at Hospital Clinic Barcelona. 2) Cohort 2, 88 patients with neoadjuvant trastuzumab and lapatinib without chemotherapy in the context of the PAMELA phase II clinical trial. 3) Cohort 3, 67 patients with HER2+ breast cancer treated with neoadjuvant anti-HER2-based chemotherapy at Hospital Clinic Barcelona (n=30) and Padova University (n=37).

For each pair of genes, a combination score was determined by calculating the ratio of the expression of the 2 genes, as follows:

Combination score=gene 1 mRNA level(log 2 value)−gene 2 mRNA level(log 2 value)

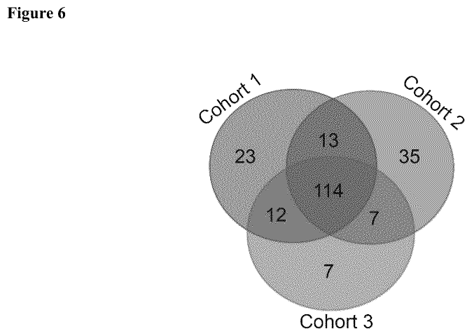

Univariate logistic regression models for pCR were used to test the ability of each combination score to predict pCR. As proof of concept, we identified several pairs significantly associated with prediction of response (pCR) in 2 or more datasets (FIG. 6 and Table 9).

TABLE 9A
List of 146 combination scores significantly associated with pCR across the 3 datasets.

| | | | | 95% CI | 95% CI | |
|---|---|---|---|---|---|---|
| | | | Odds | lower | higher | |
| Gene combination | signatures | Dataset | ratio | limit | limit | p-value |
| ERBB2_HLA.C | HER2_IGG | Cohort 2 | 1.740 | 1.052 | 3.063 | 0.04058 |
| | | Cohort 1 | 1.860 | 1.265 | 2.827 | 0.00234 |
| | | Cohort 3 | 1.911 | 1.146 | 3.364 | 0.01730 |
| ERBB2_NTN3 | HER2_IGG | Cohort 1 | 2.164 | 1.450 | 3.363 | 0.00030 |
| | | Cohort 3 | 2.369 | 1.352 | 4.566 | 0.00504 |
| GRB7_CXCL8 | HER2_IGG | Cohort 1 | 1.500 | 1.034 | 2.239 | 0.03802 |
| | | Cohort 3 | 1.698 | 1.027 | 2.954 | 0.04697 |
| GRB7_HLA.C | HER2_IGG | Cohort 3 | 1.839 | 1.106 | 3.224 | 0.02412 |
| | | Cohort 1 | 1.991 | 1.348 | 3.046 | 0.00087 |
| GRB7_NTN3 | HER2_IGG | Cohort 3 | 2.144 | 1.250 | 3.991 | 0.00932 |
| | | Cohort 1 | 2.310 | 1.538 | 3.625 | 0.00012 |
| STARD3_NTN3 | HER2_IGG | Cohort 3 | 1.878 | 1.108 | 3.458 | 0.02818 |
| | | Cohort 1 | 2.092 | 1.401 | 3.267 | 0.00058 |
| TCAP_HLA.C | HER2_IGG | Cohort 2 | 1.661 | 1.033 | 2.840 | 0.04539 |
| | | Cohort 3 | 1.861 | 1.121 | 3.238 | 0.02046 |
| TCAP_NTN3 | HER2_IGG | Cohort 1 | 1.500 | 1.037 | 2.219 | 0.03543 |
| | | Cohort 2 | 1.780 | 1.098 | 3.071 | 0.02597 |
| | | Cohort 3 | 2.247 | 1.309 | 4.216 | 0.00595 |
| ERBB2_AFF3 | HER2_LUM | Cohort 1 | 2.243 | 1.498 | 3.508 | 0.00018 |
| | | Cohort 3 | 2.791 | 1.574 | 5.503 | 0.00112 |
| | | Cohort 2 | 3.861 | 2.044 | 8.395 | 0.00015 |
| ERBB2_AGR3 | HER2_LUM | Cohort 1 | 2.274 | 1.518 | 3.554 | 0.00014 |
| | | Cohort 2 | 3.037 | 1.765 | 5.666 | 0.00016 |
| | | Cohort 3 | 3.440 | 1.867 | 7.184 | 0.00027 |
| ERBB2_BCL2 | HER2_LUM | Cohort 1 | 2.517 | 1.661 | 4.000 | 0.00003 |
| | | Cohort 3 | 2.913 | 1.631 | 5.770 | 0.00080 |
| | | Cohort 2 | 3.450 | 1.736 | 8.188 | 0.00162 |
| ERBB2_DNAJC12 | HER2_LUM | Cohort 3 | 2.294 | 1.345 | 4.204 | 0.00390 |
| | | Cohort 2 | 2.883 | 1.542 | 6.225 | 0.00266 |
| | | Cohort 1 | 2.991 | 1.907 | 5.016 | 0.00001 |
| ERBB2_ESR1 | HER2_LUM | Cohort 1 | 2.464 | 1.639 | 3.862 | 0.00003 |
| | | Cohort 3 | 3.071 | 1.726 | 6.026 | 0.00037 |
| | | Cohort 2 | 4.453 | 2.273 | 10.439 | 0.00010 |
| GRB7_AFF3 | HER2_LUM | Cohort 1 | 2.357 | 1.565 | 3.718 | 0.00009 |
| | | Cohort 3 | 2.577 | 1.468 | 5.001 | 0.00217 |
| | | Cohort 2 | 3.003 | 1.698 | 5.893 | 0.00046 |
| GRB7_AGR3 | HER2_LUM | Cohort 1 | 2.383 | 1.582 | 3.753 | 0.00007 |
| | | Cohort 2 | 2.755 | 1.621 | 5.046 | 0.00041 |
| | | Cohort 3 | 3.238 | 1.779 | 6.633 | 0.00039 |
| GRB7_BCL2 | HER2_LUM | Cohort 2 | 2.467 | 1.383 | 4.921 | 0.00492 |
| | | Cohort 1 | 2.662 | 1.748 | 4.255 | 0.00001 |
| | | Cohort 3 | 2.678 | 1.518 | 5.220 | 0.00156 |
| GRB7_DNAJC12 | HER2_LUM | Cohort 2 | 2.165 | 1.261 | 4.078 | 0.00913 |
| | | Cohort 3 | 2.201 | 1.296 | 4.002 | 0.00558 |
| | | Cohort 1 | 3.136 | 1.990 | 5.280 | 0.00000 |
| GRB7_ESR1 | HER2_LUM | Cohort 1 | 2.553 | 1.691 | 4.025 | 0.00002 |
| | | Cohort 3 | 2.902 | 1.641 | 5.642 | 0.00062 |
| | | Cohort 2 | 3.452 | 1.894 | 7.148 | 0.00021 |
| STARD3_AFF3 | HER2_LUM | Cohort 1 | 2.305 | 1.530 | 3.643 | 0.00014 |
| | | Cohort 3 | 2.635 | 1.491 | 5.177 | 0.00199 |
| | | Cohort 2 | 3.248 | 1.836 | 6.356 | 0.00017 |
| STARD3_AGR3 | HER2_LUM | Cohort 1 | 2.218 | 1.486 | 3.445 | 0.00019 |
| | | Cohort 2 | 2.697 | 1.596 | 4.905 | 0.00047 |
| | | Cohort 3 | 3.424 | 1.854 | 7.189 | 0.00031 |
| STARD3_BCL2 | HER2_LUM | Cohort 2 | 2.536 | 1.448 | 4.896 | 0.00250 |
| | | Cohort 3 | 2.715 | 1.542 | 5.254 | 0.00124 |
| | | Cohort 1 | 2.767 | 1.786 | 4.550 | 0.00002 |
| STARD3_DNAJC12 | HER2_LUM | Cohort 3 | 2.115 | 1.250 | 3.829 | 0.00798 |
| | | Cohort 2 | 2.230 | 1.292 | 4.198 | 0.00715 |
| | | Cohort 1 | 3.150 | 1.979 | 5.402 | 0.00001 |
| STARD3_ESR1 | HER2_LUM | Cohort 1 | 2.512 | 1.666 | 3.952 | 0.00003 |
| | | Cohort 3 | 2.998 | 1.690 | 5.859 | 0.00046 |
| | | Cohort 2 | 3.960 | 2.120 | 8.458 | 0.00008 |
| TCAP_AFF3 | HER2_LUM | Cohort 1 | 1.897 | 1.287 | 2.893 | 0.00182 |
| | | Cohort 3 | 2.639 | 1.500 | 5.125 | 0.00173 |
| | | Cohort 2 | 4.568 | 2.379 | 10.189 | 0.00003 |
| TCAP_AGR3 | HER2_LUM | Cohort 1 | 1.920 | 1.305 | 2.916 | 0.00137 |
| | | Cohort 2 | 3.129 | 1.805 | 5.942 | 0.00015 |
| | | Cohort 3 | 3.245 | 1.781 | 6.661 | 0.00040 |
| TCAP_BCL2 | HER2_LUM | Cohort 1 | 2.111 | 1.411 | 3.303 | 0.00053 |
| | | Cohort 3 | 2.939 | 1.645 | 5.824 | 0.00073 |
| | | Cohort 2 | 3.556 | 1.896 | 7.675 | 0.00033 |
| TCAP_DNAJC12 | HER2_LUM | Cohort 3 | 2.271 | 1.325 | 4.207 | 0.00488 |
| | | Cohort 1 | 2.395 | 1.574 | 3.850 | 0.00012 |
| | | Cohort 2 | 3.017 | 1.647 | 6.244 | 0.00105 |
| TCAP_ESR1 | HER2_LUM | Cohort 1 | 2.145 | 1.446 | 3.294 | 0.00026 |
| | | Cohort 3 | 2.990 | 1.687 | 5.839 | 0.00047 |
| | | Cohort 2 | 5.296 | 2.573 | 13.484 | 0.00006 |
| ERBB2_ASPM | HER2_PROLIF | Cohort 1 | 1.507 | 1.040 | 2.245 | 0.03519 |
| | | Cohort 3 | 1.792 | 1.078 | 3.147 | 0.03096 |
| ERBB2_EXO1 | HER2_PROLIF | Cohort 1 | 1.598 | 1.100 | 2.385 | 0.01676 |
| | | Cohort 3 | 1.680 | 1.019 | 2.892 | 0.04878 |
| | | Cohort 2 | 1.943 | 1.142 | 3.627 | 0.02271 |
| ERBB2_KIF23 | HER2_PROLIF | Cohort 1 | 1.627 | 1.117 | 2.442 | 0.01399 |
| | | Cohort 2 | 1.871 | 1.097 | 3.524 | 0.03332 |
| | | Cohort 3 | 2.080 | 1.235 | 3.727 | 0.00867 |
| ERBB2_NEK2 | HER2_PROLIF | Cohort 1 | 1.632 | 1.124 | 2.434 | 0.01235 |
| | | Cohort 2 | 1.973 | 1.143 | 3.788 | 0.02449 |
| | | Cohort 3 | 2.179 | 1.282 | 3.976 | 0.00638 |
| GRB7_ASPM | HER2_PROLIF | Cohort 1 | 1.647 | 1.129 | 2.484 | 0.01241 |
| | | Cohort 3 | 1.712 | 1.035 | 2.974 | 0.04338 |
| GRB7_KIF23 | HER2_PROLIF | Cohort 1 | 1.781 | 1.215 | 2.698 | 0.00428 |
| | | Cohort 3 | 1.954 | 1.165 | 3.476 | 0.01525 |
| GRB7_NEK2 | HER2_PROLIF | Cohort 1 | 1.770 | 1.211 | 2.668 | 0.00434 |
| | | Cohort 3 | 2.050 | 1.217 | 3.676 | 0.01010 |
| STARD3_KIF23 | HER2_PROLIF | Cohort 1 | 1.474 | 1.021 | 2.171 | 0.04271 |
| | | Cohort 3 | 1.779 | 1.071 | 3.121 | 0.03285 |
| STARD3_NEK2 | HER2_PROLIF | Cohort 1 | 1.494 | 1.034 | 2.202 | 0.03629 |
| | | Cohort 3 | 1.898 | 1.130 | 3.409 | 0.02135 |
| TCAP_EXO1 | HER2_PROLIF | Cohort 3 | 1.669 | 1.015 | 2.848 | 0.04950 |
| | | Cohort 2 | 1.952 | 1.185 | 3.459 | 0.01328 |
| TCAP_KIF23 | HER2_PROLIF | Cohort 2 | 1.815 | 1.117 | 3.160 | 0.02283 |
| | | Cohort 3 | 1.996 | 1.190 | 3.557 | 0.01242 |
| TCAP_NEK2 | HER2_PROLIF | Cohort 2 | 1.915 | 1.160 | 3.440 | 0.01771 |
| | | Cohort 3 | 2.071 | 1.230 | 3.709 | 0.00896 |
| IGKC_HLA.C | IGG_IGG | Cohort 1 | 1.519 | 1.048 | 2.262 | 0.03179 |
| | | Cohort 2 | 1.676 | 1.029 | 2.865 | 0.04592 |
| IGL_CD27 | IGG_IGG | Cohort 1 | 1.510 | 1.040 | 2.253 | 0.03545 |
| | | Cohort 2 | 2.709 | 1.569 | 5.197 | 0.00099 |
| IGL_HLA.C | IGG_IGG | Cohort 1 | 1.631 | 1.117 | 2.458 | 0.01426 |
| | | Cohort 2 | 2.649 | 1.534 | 5.009 | 0.00112 |
| IGL_IGJ | IGG_IGG | Cohort 1 | 1.544 | 1.062 | 2.311 | 0.02744 |
| | | Cohort 2 | 3.013 | 1.695 | 6.058 | 0.00062 |
| IGL_LAX1 | IGG_IGG | Cohort 1 | 1.479 | 1.021 | 2.196 | 0.04370 |
| | | Cohort 2 | 2.249 | 1.334 | 4.123 | 0.00441 |
| IGL_NTN3 | IGG_IGG | Cohort 1 | 1.458 | 1.010 | 2.149 | 0.04886 |
| | | Cohort 2 | 2.185 | 1.313 | 3.885 | 0.00433 |
| IGL_PIM2 | IGG_IGG | Cohort 1 | 1.600 | 1.095 | 2.415 | 0.01903 |
| | | Cohort 2 | 2.746 | 1.561 | 5.440 | 0.00134 |
| IGL_POU2AF1 | IGG_IGG | Cohort 1 | 1.585 | 1.086 | 2.391 | 0.02127 |
| | | Cohort 2 | 2.072 | 1.242 | 3.749 | 0.00889 |
| IGL_TNFRSF17 | IGG_IGG | Cohort 1 | 1.519 | 1.046 | 2.266 | 0.03307 |
| | | Cohort 2 | 2.082 | 1.243 | 3.780 | 0.00893 |
| LAX1_HLA.C | IGG_IGG | Cohort 1 | 1.487 | 1.026 | 2.211 | 0.04140 |
| | | Cohort 2 | 1.680 | 1.048 | 2.833 | 0.03831 |
| | | Cohort 3 | 1.721 | 1.031 | 3.084 | 0.04877 |
| CD27_AFF3 | IGG_LUM | Cohort 1 | 1.982 | 1.338 | 3.050 | 0.00105 |
| | | Cohort 3 | 2.389 | 1.378 | 4.501 | 0.00358 |
| | | Cohort 2 | 3.918 | 2.009 | 9.040 | 0.00031 |
| CD27_AGR3 | IGG_LUM | Cohort 1 | 1.874 | 1.276 | 2.836 | 0.00192 |
| | | Cohort 2 | 2.461 | 1.472 | 4.387 | 0.00110 |
| | | Cohort 3 | 3.046 | 1.712 | 5.965 | 0.00041 |
| CD27_BCL2 | IGG_LUM | Cohort 1 | 2.768 | 1.761 | 4.630 | 0.00003 |
| | | Cohort 3 | 3.303 | 1.765 | 7.165 | 0.00070 |
| | | Cohort 2 | 3.563 | 1.800 | 8.080 | 0.00089 |
| CD27_DNAJC12 | IGG_LUM | Cohort 2 | 1.959 | 1.155 | 3.616 | 0.01974 |
| | | Cohort 3 | 2.010 | 1.188 | 3.655 | 0.01386 |
| | | Cohort 1 | 2.609 | 1.684 | 4.304 | 0.00006 |
| CD27_ESR1 | IGG_LUM | Cohort 1 | 2.380 | 1.582 | 3.735 | 0.00007 |
| | | Cohort 3 | 2.895 | 1.645 | 5.565 | 0.00055 |
| | | Cohort 2 | 4.108 | 2.110 | 9.599 | 0.00020 |
| CD79A_AFF3 | IGG_LUM | Cohort 1 | 1.929 | 1.304 | 2.969 | 0.00162 |
| | | Cohort 3 | 2.140 | 1.254 | 3.937 | 0.00848 |
| | | Cohort 2 | 3.394 | 1.835 | 7.157 | 0.00037 |
| CD79A_AGR3 | IGG_LUM | Cohort 1 | 1.929 | 1.306 | 2.955 | 0.00150 |
| | | Cohort 2 | 2.504 | 1.487 | 4.528 | 0.00110 |
| | | Cohort 3 | 2.701 | 1.546 | 5.126 | 0.00104 |
| CD79A_BCL2 | IGG_LUM | Cohort 1 | 2.211 | 1.471 | 3.469 | 0.00027 |
| | | Cohort 3 | 2.398 | 1.380 | 4.567 | 0.00375 |
| | | Cohort 2 | 2.502 | 1.461 | 4.649 | 0.00171 |
| CD79A_DNAJC12 | IGG_LUM | Cohort 3 | 1.824 | 1.093 | 3.235 | 0.02819 |
| | | Cohort 2 | 1.989 | 1.195 | 3.548 | 0.01231 |
| | | Cohort 1 | 2.287 | 1.513 | 3.627 | 0.00019 |
| CD79A_ESR1 | IGG_LUM | Cohort 1 | 2.365 | 1.562 | 3.758 | 0.00011 |
| | | Cohort 3 | 2.596 | 1.497 | 4.878 | 0.00139 |
| | | Cohort 2 | 3.509 | 1.922 | 7.250 | 0.00017 |
| CXCL8_AFF3 | IGG_LUM | Cohort 1 | 1.716 | 1.174 | 2.587 | 0.00697 |
| | | Cohort 3 | 2.027 | 1.198 | 3.689 | 0.01271 |
| | | Cohort 2 | 3.687 | 1.991 | 7.794 | 0.00015 |
| CXCL8_AGR3 | IGG_LUM | Cohort 1 | 1.732 | 1.187 | 2.593 | 0.00561 |
| | | Cohort 3 | 2.620 | 1.509 | 4.936 | 0.00129 |
| | | Cohort 2 | 2.813 | 1.661 | 5.155 | 0.00030 |
| CXCL8_BCL2 | IGG_LUM | Cohort 1 | 1.878 | 1.269 | 2.895 | 0.00256 |
| | | Cohort 3 | 2.088 | 1.215 | 3.938 | 0.01293 |
| | | Cohort 2 | 3.122 | 1.751 | 6.217 | 0.00037 |
| CXCL8_DNAJC12 | IGG_LUM | Cohort 3 | 1.724 | 1.041 | 3.006 | 0.04178 |
| | | Cohort 1 | 2.149 | 1.427 | 3.401 | 0.00051 |
| | | Cohort 2 | 3.273 | 1.705 | 7.230 | 0.00120 |
| CXCL8_ESR1 | IGG_LUM | Cohort 1 | 1.986 | 1.347 | 3.023 | 0.00082 |
| | | Cohort 3 | 2.420 | 1.414 | 4.440 | 0.00224 |
| | | Cohort 2 | 4.156 | 2.161 | 9.413 | 0.00012 |
| HLA.C_AFF3 | IGG_LUM | Cohort 1 | 1.707 | 1.171 | 2.554 | 0.00688 |
| | | Cohort 3 | 1.984 | 1.175 | 3.596 | 0.01516 |
| | | Cohort 2 | 3.324 | 1.817 | 6.937 | 0.00038 |
| HLA.C_AGR3 | IGG_LUM | Cohort 1 | 1.739 | 1.194 | 2.597 | 0.00499 |
| | | Cohort 2 | 2.457 | 1.467 | 4.411 | 0.00122 |
| | | Cohort 3 | 2.755 | 1.574 | 5.244 | 0.00086 |
| HLA.C_BCL2 | IGG_LUM | Cohort 1 | 2.014 | 1.361 | 3.087 | 0.00075 |
| | | Cohort 3 | 2.151 | 1.246 | 4.032 | 0.00990 |
| | | Cohort 2 | 2.951 | 1.560 | 6.587 | 0.00290 |
| HLA.C_DNAJC12 | IGG_LUM | Cohort 2 | 1.931 | 1.137 | 3.591 | 0.02339 |
| | | Cohort 1 | 2.301 | 1.520 | 3.672 | 0.00019 |
| HLA.C_ESR1 | IGG_LUM | Cohort 1 | 2.142 | 1.447 | 3.278 | 0.00024 |
| | | Cohort 3 | 2.505 | 1.461 | 4.605 | 0.00154 |
| | | Cohort 2 | 4.239 | 2.176 | 9.843 | 0.00014 |
| IGJ_AFF3 | IGG_LUM | Cohort 1 | 1.792 | 1.221 | 2.720 | 0.00402 |
| | | Cohort 3 | 2.191 | 1.242 | 4.273 | 0.01223 |
| | | Cohort 2 | 3.314 | 1.762 | 7.130 | 0.00070 |
| IGJ_AGR3 | IGG_LUM | Cohort 1 | 1.818 | 1.239 | 2.754 | 0.00315 |
| | | Cohort 2 | 2.453 | 1.448 | 4.468 | 0.00164 |
| | | Cohort 3 | 2.948 | 1.641 | 5.821 | 0.00074 |
| IGJ_BCL2 | IGG_LUM | Cohort 1 | 2.155 | 1.428 | 3.410 | 0.00051 |
| | | Cohort 3 | 2.270 | 1.266 | 4.655 | 0.01258 |
| | | Cohort 2 | 2.307 | 1.324 | 4.398 | 0.00597 |
| IGJ_DNAJC12 | IGG_LUM | Cohort 3 | 1.776 | 1.055 | 3.225 | 0.04151 |
| | | Cohort 2 | 1.846 | 1.106 | 3.307 | 0.02674 |
| | | Cohort 1 | 2.371 | 1.563 | 3.782 | 0.00012 |
| IGJ_ESR1 | IGG_LUM | Cohort 1 | 2.253 | 1.500 | 3.532 | 0.00018 |
| | | Cohort 3 | 2.721 | 1.522 | 5.364 | 0.00167 |
| | | Cohort 2 | 3.352 | 1.821 | 6.962 | 0.00035 |
| IGKC_AFF3 | IGG_LUM | Cohort 1 | 1.921 | 1.297 | 2.970 | 0.00186 |
| | | Cohort 3 | 2.099 | 1.198 | 4.055 | 0.01639 |
| | | Cohort 2 | 3.476 | 1.858 | 7.443 | 0.00037 |
| IGKC_AGR3 | IGG_LUM | Cohort 1 | 1.937 | 1.311 | 2.972 | 0.00143 |
| | | Cohort 2 | 2.636 | 1.542 | 4.868 | 0.00085 |
| | | Cohort 3 | 2.812 | 1.567 | 5.536 | 0.00121 |
| IGKC_BCL2 | IGG_LUM | Cohort 3 | 2.215 | 1.248 | 4.379 | 0.01247 |
| | | Cohort 1 | 2.218 | 1.469 | 3.511 | 0.00031 |
| | | Cohort 2 | 2.800 | 1.566 | 5.508 | 0.00124 |
| IGKC_DNAJC12 | IGG_LUM | Cohort 3 | 1.790 | 1.059 | 3.271 | 0.04102 |
| | | Cohort 2 | 2.203 | 1.288 | 4.103 | 0.00697 |
| | | Cohort 1 | 2.398 | 1.565 | 3.896 | 0.00015 |
| IGKC_ESR1 | IGG_LUM | Cohort 1 | 2.394 | 1.573 | 3.843 | 0.00011 |
| | | Cohort 3 | 2.557 | 1.448 | 4.934 | 0.00248 |
| | | Cohort 2 | 3.772 | 2.012 | 8.113 | 0.00016 |
| IGL_AFF3 | IGG_LUM | Cohort 1 | 2.011 | 1.352 | 3.122 | 0.00098 |
| | | Cohort 3 | 2.092 | 1.223 | 3.877 | 0.01130 |
| | | Cohort 2 | 4.654 | 2.373 | 10.857 | 0.00006 |
| IGL_AGR3 | IGG_LUM | Cohort 1 | 2.045 | 1.373 | 3.175 | 0.00076 |
| | | Cohort 3 | 2.817 | 1.590 | 5.474 | 0.00092 |
| | | Cohort 2 | 3.319 | 1.877 | 6.508 | 0.00013 |
| IGL_BCL2 | IGG_LUM | Cohort 3 | 2.215 | 1.283 | 4.205 | 0.00781 |
| | | Cohort 1 | 2.260 | 1.496 | 3.580 | 0.00023 |
| | | Cohort 2 | 3.928 | 2.087 | 8.538 | 0.00012 |
| IGL_DNAJC12 | IGG_LUM | Cohort 3 | 1.825 | 1.090 | 3.269 | 0.02987 |
| | | Cohort 1 | 2.446 | 1.593 | 3.988 | 0.00012 |
| | | Cohort 2 | 2.984 | 1.675 | 5.963 | 0.00064 |
| IGL_ESR1 | IGG_LUM | Cohort 1 | 2.426 | 1.594 | 3.888 | 0.00009 |
| | | Cohort 3 | 2.612 | 1.495 | 4.981 | 0.00158 |
| | | Cohort 2 | 5.184 | 2.580 | 12.565 | 0.00004 |
| IGLV3.25_AFF3 | IGG_LUM | Cohort 1 | 1.784 | 1.217 | 2.703 | 0.00422 |
| | | Cohort 3 | 1.846 | 1.104 | 3.286 | 0.02589 |
| | | Cohort 2 | 2.976 | 1.691 | 5.866 | 0.00050 |
| IGLV3.25_AGR3 | IGG_LUM | Cohort 1 | 1.850 | 1.258 | 2.813 | 0.00255 |
| | | Cohort 2 | 2.483 | 1.490 | 4.477 | 0.00103 |
| | | Cohort 3 | 2.510 | 1.444 | 4.753 | 0.00222 |
| IGLV3.25_BCL2 | IGG_LUM | Cohort 3 | 1.826 | 1.093 | 3.254 | 0.02842 |
| | | Cohort 1 | 1.837 | 1.250 | 2.789 | 0.00281 |
| | | Cohort 2 | 2.195 | 1.335 | 3.860 | 0.00335 |
| IGLV3.25_DNAJC12 | IGG_LUM | Cohort 2 | 2.003 | 1.228 | 3.467 | 0.00793 |
| | | Cohort 1 | 2.054 | 1.380 | 3.180 | 0.00067 |
| IGLV3.25_ESR1 | IGG_LUM | Cohort 1 | 2.137 | 1.431 | 3.330 | 0.00039 |
| | | Cohort 3 | 2.250 | 1.325 | 4.084 | 0.00432 |
| | | Cohort 2 | 3.300 | 1.837 | 6.738 | 0.00026 |
| IL2RG_AFF3 | IGG_LUM | Cohort 1 | 2.057 | 1.377 | 3.216 | 0.00079 |
| | | Cohort 3 | 2.301 | 1.325 | 4.373 | 0.00569 |
| | | Cohort 2 | 3.172 | 1.728 | 6.632 | 0.00066 |
| IL2RG_AGR3 | IGG_LUM | Cohort 1 | 1.954 | 1.322 | 2.990 | 0.00121 |
| | | Cohort 2 | 2.286 | 1.382 | 4.005 | 0.00211 |
| | | Cohort 3 | 3.141 | 1.747 | 6.271 | 0.00038 |
| IL2RG_BCL2 | IGG_LUM | Cohort 3 | 2.590 | 1.472 | 5.019 | 0.00210 |
| | | Cohort 2 | 2.625 | 1.437 | 5.445 | 0.00423 |
| | | Cohort 1 | 2.741 | 1.741 | 4.606 | 0.00004 |
| IL2RG_DNAJC12 | IGG_LUM | Cohort 2 | 1.839 | 1.095 | 3.351 | 0.03102 |
| | | Cohort 3 | 1.900 | 1.128 | 3.443 | 0.02250 |
| | | Cohort 1 | 2.615 | 1.679 | 4.349 | 0.00007 |
| IL2RG_ESR1 | IGG_LUM | Cohort 1 | 2.458 | 1.621 | 3.907 | 0.00006 |
| | | Cohort 3 | 2.909 | 1.641 | 5.671 | 0.00065 |
| | | Cohort 2 | 3.363 | 1.836 | 7.010 | 0.00032 |
| LAX1_AFF3 | IGG_LUM | Cohort 1 | 1.943 | 1.312 | 2.988 | 0.00145 |
| | | Cohort 3 | 2.283 | 1.320 | 4.296 | 0.00562 |
| | | Cohort 2 | 4.253 | 2.166 | 9.911 | 0.00016 |
| LAX1_AGR3 | IGG_LUM | Cohort 1 | 1.892 | 1.287 | 2.869 | 0.00171 |
| | | Cohort 2 | 2.735 | 1.615 | 4.991 | 0.00041 |
| | | Cohort 3 | 2.993 | 1.685 | 5.837 | 0.00047 |
| LAX1_BCL2 | IGG_LUM | Cohort 1 | 2.526 | 1.637 | 4.119 | 0.00008 |
| | | Cohort 3 | 2.836 | 1.564 | 5.852 | 0.00169 |
| | | Cohort 2 | 3.777 | 2.001 | 8.114 | 0.00017 |
| LAX1_DNAJC12 | IGG_LUM | Cohort 3 | 1.918 | 1.136 | 3.491 | 0.02124 |
| | | Cohort 2 | 2.440 | 1.395 | 4.711 | 0.00371 |
| | | Cohort 1 | 2.581 | 1.667 | 4.245 | 0.00006 |
| LAX1_ESR1 | IGG_LUM | Cohort 1 | 2.341 | 1.557 | 3.671 | 0.00009 |
| | | Cohort 3 | 2.788 | 1.590 | 5.331 | 0.00079 |
| | | Cohort 2 | 4.615 | 2.314 | 11.252 | 0.00011 |
| NTN3_AFF3 | IGG_LUM | Cohort 1 | 1.684 | 1.157 | 2.518 | 0.00822 |
| | | Cohort 3 | 2.018 | 1.194 | 3.658 | 0.01287 |
| | | Cohort 2 | 3.202 | 1.774 | 6.473 | 0.00037 |
| NTN3_AGR3 | IGG_LUM | Cohort 1 | 1.701 | 1.171 | 2.526 | 0.00647 |
| | | Cohort 2 | 2.504 | 1.494 | 4.483 | 0.00095 |
| | | Cohort 3 | 2.691 | 1.541 | 5.129 | 0.00110 |
| NTN3_BCL2 | IGG_LUM | Cohort 1 | 1.994 | 1.346 | 3.071 | 0.00096 |
| | | Cohort 3 | 2.261 | 1.302 | 4.349 | 0.00718 |
| | | Cohort 2 | 3.061 | 1.692 | 6.149 | 0.00061 |
| NTN3_DNAJC12 | IGG_LUM | Cohort 2 | 2.100 | 1.212 | 4.039 | 0.01468 |
| | | Cohort 1 | 2.362 | 1.552 | 3.801 | 0.00015 |
| NTN3_ESR1 | IGG_LUM | Cohort 1 | 2.065 | 1.402 | 3.136 | 0.00039 |
| | | Cohort 3 | 2.493 | 1.451 | 4.608 | 0.00175 |
| | | Cohort 2 | 3.802 | 2.007 | 8.443 | 0.00021 |
| PIM2_AFF3 | IGG_LUM | Cohort 1 | 1.908 | 1.295 | 2.908 | 0.00162 |
| | | Cohort 3 | 2.115 | 1.247 | 3.843 | 0.00836 |
| | | Cohort 2 | 3.874 | 2.061 | 8.385 | 0.00013 |
| PIM2_AGR3 | IGG_LUM | Cohort 1 | 1.861 | 1.267 | 2.819 | 0.00219 |
| | | Cohort 2 | 2.691 | 1.596 | 4.869 | 0.00045 |
| | | Cohort 3 | 2.807 | 1.599 | 5.379 | 0.00075 |
| PIM2_BCL2 | IGG_LUM | Cohort 1 | 2.348 | 1.559 | 3.696 | 0.00010 |
| | | Cohort 3 | 2.452 | 1.406 | 4.712 | 0.00324 |
| | | Cohort 2 | 3.217 | 1.811 | 6.327 | 0.00022 |
| PIM2_DNAJC12 | IGG_LUM | Cohort 3 | 1.792 | 1.078 | 3.149 | 0.03101 |
| | | Cohort 2 | 2.285 | 1.342 | 4.242 | 0.00442 |
| | | Cohort 1 | 2.453 | 1.610 | 3.941 | 0.00008 |
| PIM2_ESR1 | IGG_LUM | Cohort 1 | 2.332 | 1.555 | 3.645 | 0.00009 |
| | | Cohort 3 | 2.634 | 1.521 | 4.934 | 0.00112 |
| | | Cohort 2 | 4.345 | 2.260 | 9.840 | 0.00007 |
| POU2AF1_AFF3 | IGG_LUM | Cohort 1 | 1.819 | 1.237 | 2.775 | 0.00347 |
| | | Cohort 3 | 2.138 | 1.256 | 3.912 | 0.00814 |
| | | Cohort 2 | 4.304 | 2.196 | 10.007 | 0.00013 |
| POU2AF1_AGR3 | IGG_LUM | Cohort 1 | 1.809 | 1.234 | 2.734 | 0.00328 |
| | | Cohort 3 | 2.693 | 1.545 | 5.101 | 0.00102 |
| | | Cohort 2 | 2.801 | 1.641 | 5.169 | 0.00038 |
| POU2AF1_BCL2 | IGG_LUM | Cohort 1 | 2.126 | 1.417 | 3.337 | 0.00051 |
| | | Cohort 3 | 2.461 | 1.409 | 4.721 | 0.00316 |
| | | Cohort 2 | 3.388 | 1.882 | 6.817 | 0.00017 |
| POU2AF1_DNAJC12 | IGG_LUM | Cohort 3 | 1.834 | 1.099 | 3.253 | 0.02661 |
| | | Cohort 1 | 2.246 | 1.486 | 3.567 | 0.00027 |
| | | Cohort 2 | 2.373 | 1.388 | 4.423 | 0.00312 |
| POU2AF1_ESR1 | IGG_LUM | Cohort 1 | 2.220 | 1.484 | 3.462 | 0.00021 |
| | | Cohort 3 | 2.557 | 1.483 | 4.754 | 0.00142 |
| | | Cohort 2 | 4.491 | 2.305 | 10.380 | 0.00007 |
| TNFRSF17_AFF3 | IGG_LUM | Cohort 1 | 1.929 | 1.305 | 2.960 | 0.00156 |
| | | Cohort 3 | 2.044 | 1.207 | 3.715 | 0.01170 |
| | | Cohort 2 | 4.519 | 2.287 | 10.661 | 0.00010 |
| TNFRSF17_AGR3 | IGG_LUM | Cohort 1 | 1.878 | 1.279 | 2.843 | 0.00187 |
| | | Cohort 3 | 2.666 | 1.535 | 5.013 | 0.00103 |
| | | Cohort 2 | 2.832 | 1.661 | 5.223 | 0.00032 |
| TNFRSF17_BCL2 | IGG_LUM | Cohort 3 | 2.336 | 1.333 | 4.549 | 0.00608 |
| | | Cohort 1 | 2.435 | 1.595 | 3.916 | 0.00009 |
| | | Cohort 2 | 3.504 | 1.933 | 7.108 | 0.00014 |
| TNFRSF17_DNAJC12 | IGG_LUM | Cohort 3 | 1.724 | 1.039 | 3.032 | 0.04356 |
| | | Cohort 2 | 2.458 | 1.429 | 4.623 | 0.00241 |
| | | Cohort 1 | 2.573 | 1.666 | 4.225 | 0.00006 |
| TNFRSF17_ESR1 | IGG_LUM | Cohort 1 | 2.338 | 1.558 | 3.658 | 0.00009 |
| | | Cohort 3 | 2.492 | 1.450 | 4.610 | 0.00178 |
| | | Cohort 2 | 4.715 | 2.373 | 11.338 | 0.00008 |
| AFF3_ESR1 | LUM_LUM | Cohort 1 | 1.673 | 1.141 | 2.547 | 0.01135 |
| | | Cohort 3 | 1.733 | 1.041 | 3.071 | 0.04385 |
| BCL2_AGR3 | LUM_LUM | Cohort 2 | 1.820 | 1.116 | 3.103 | 0.02058 |
| | | Cohort 3 | 2.251 | 1.323 | 4.107 | 0.00454 |
| BCL2_ESR1 | LUM_LUM | Cohort 1 | 1.738 | 1.192 | 2.599 | 0.00520 |
| | | Cohort 3 | 2.123 | 1.262 | 3.778 | 0.00659 |
| | | Cohort 2 | 2.927 | 1.601 | 6.082 | 0.00142 |
| DNAJC12_AGR3 | LUM_LUM | Cohort 2 | 1.811 | 1.115 | 3.087 | 0.02080 |
| | | Cohort 3 | 2.580 | 1.471 | 4.966 | 0.00202 |
| DNAJC12_ESR1 | LUM_LUM | Cohort 2 | 2.253 | 1.312 | 4.235 | 0.00603 |
| | | Cohort 3 | 2.288 | 1.328 | 4.299 | 0.00519 |
| ASPM_AFF3 | PROLIF_LUM | Cohort 1 | 2.147 | 1.435 | 3.356 | 0.00039 |
| | | Cohort 3 | 2.330 | 1.350 | 4.391 | 0.00442 |
| | | Cohort 2 | 3.625 | 1.944 | 7.673 | 0.00020 |
| ASPM_AGR3 | PROLIF_LUM | Cohort 1 | 2.036 | 1.377 | 3.113 | 0.00059 |
| | | Cohort 2 | 2.597 | 1.552 | 4.637 | 0.00056 |
| | | Cohort 3 | 3.458 | 1.856 | 7.424 | 0.00037 |
| ASPM_BCL2 | PROLIF_LUM | Cohort 3 | 2.399 | 1.383 | 4.554 | 0.00358 |
| | | Cohort 1 | 2.671 | 1.726 | 4.396 | 0.00003 |
| | | Cohort 2 | 3.367 | 1.754 | 7.408 | 0.00087 |
| ASPM_DNAJC12 | PROLIF_LUM | Cohort 3 | 1.934 | 1.151 | 3.472 | 0.01792 |
| | | Cohort 2 | 2.346 | 1.292 | 4.837 | 0.01088 |
| | | Cohort 1 | 2.906 | 1.848 | 4.903 | 0.00002 |
| ASPM_ESR1 | PROLIF_LUM | Cohort 1 | 2.439 | 1.621 | 3.829 | 0.00004 |
| | | Cohort 3 | 3.227 | 1.782 | 6.512 | 0.00034 |
| | | Cohort 2 | 5.082 | 2.447 | 13.141 | 0.00011 |
| EXO1_AFF3 | PROLIF_LUM | Cohort 1 | 2.010 | 1.358 | 3.084 | 0.00079 |
| | | Cohort 3 | 2.453 | 1.409 | 4.694 | 0.00306 |
| | | Cohort 2 | 3.079 | 1.725 | 6.113 | 0.00043 |
| EXO1_AGR3 | PROLIF_LUM | Cohort 1 | 1.938 | 1.319 | 2.932 | 0.00109 |
| | | Cohort 2 | 2.408 | 1.453 | 4.230 | 0.00114 |
| | | Cohort 3 | 3.598 | 1.920 | 7.777 | 0.00027 |
| EXO1_BCL2 | PROLIF_LUM | Cohort 2 | 2.348 | 1.351 | 4.472 | 0.00481 |
| | | Cohort 1 | 2.460 | 1.618 | 3.938 | 0.00007 |
| | | Cohort 3 | 2.790 | 1.568 | 5.507 | 0.00120 |
| EXO1_DNAJC12 | PROLIF_LUM | Cohort 2 | 1.958 | 1.137 | 3.727 | 0.02520 |
| | | Cohort 3 | 2.078 | 1.223 | 3.810 | 0.01072 |
| | | Cohort 1 | 2.784 | 1.788 | 4.624 | 0.00002 |
| EXO1_ESR1 | PROLIF_LUM | Cohort 1 | 2.347 | 1.569 | 3.649 | 0.00007 |
| | | Cohort 3 | 3.373 | 1.853 | 6.861 | 0.00023 |
| | | Cohort 2 | 3.903 | 2.040 | 8.796 | 0.00021 |
| KIF23_AFF3 | PROLIF_LUM | Cohort 1 | 2.105 | 1.413 | 3.265 | 0.00046 |
| | | Cohort 3 | 2.198 | 1.286 | 4.063 | 0.00663 |
| | | Cohort 2 | 3.347 | 1.860 | 6.711 | 0.00019 |
| KIF23_AGR3 | PROLIF_LUM | Cohort 1 | 1.979 | 1.343 | 3.007 | 0.00084 |
| | | Cohort 2 | 2.595 | 1.553 | 4.610 | 0.00053 |
| | | Cohort 3 | 3.156 | 1.743 | 6.407 | 0.00046 |
| KIF23_BCL2 | PROLIF_LUM | Cohort 3 | 2.299 | 1.336 | 4.299 | 0.00476 |
| | | Cohort 1 | 2.740 | 1.760 | 4.552 | 0.00003 |
| | | Cohort 2 | 3.016 | 1.649 | 6.179 | 0.00096 |
| KIF23_DNAJC12 | PROLIF_LUM | Cohort 3 | 1.804 | 1.086 | 3.158 | 0.02861 |
| | | Cohort 2 | 2.160 | 1.235 | 4.191 | 0.01279 |
| | | Cohort 1 | 2.935 | 1.860 | 4.977 | 0.00002 |
| KIF23_ESR1 | PROLIF_LUM | Cohort 1 | 2.422 | 1.612 | 3.793 | 0.00005 |
| | | Cohort 3 | 2.956 | 1.669 | 5.749 | 0.00052 |
| | | Cohort 2 | 4.577 | 2.298 | 11.098 | 0.00011 |
| NEK2_AFF3 | PROLIF_LUM | Cohort 1 | 1.983 | 1.340 | 3.043 | 0.00099 |
| | | Cohort 3 | 2.071 | 1.221 | 3.788 | 0.01072 |
| | | Cohort 2 | 3.116 | 1.749 | 6.182 | 0.00036 |
| NEK2_AGR3 | PROLIF_LUM | Cohort 1 | 1.931 | 1.314 | 2.922 | 0.00116 |
| | | Cohort 2 | 2.503 | 1.502 | 4.443 | 0.00082 |
| | | Cohort 3 | 3.200 | 1.752 | 6.614 | 0.00050 |
| NEK2_BCL2 | PROLIF_LUM | Cohort 3 | 1.945 | 1.155 | 3.517 | 0.01777 |
| | | Cohort 1 | 2.422 | 1.587 | 3.900 | 0.00010 |
| | | Cohort 2 | 2.602 | 1.464 | 5.129 | 0.00257 |
| NEK2_DNAJC12 | PROLIF_LUM | Cohort 2 | 2.006 | 1.164 | 3.815 | 0.02033 |
| | | Cohort 1 | 2.769 | 1.772 | 4.633 | 0.00003 |
| NEK2_ESR1 | PROLIF_LUM | Cohort 1 | 2.368 | 1.577 | 3.706 | 0.00007 |
| | | Cohort 3 | 2.986 | 1.673 | 5.889 | 0.00057 |
| | | Cohort 2 | 4.569 | 2.282 | 11.042 | 0.00012 |
| ASPM_NEK2 | PROLIF_PROLIF | Cohort 1 | 1.514 | 1.036 | 2.298 | 0.03967 |
| | | Cohort 2 | 1.685 | 1.038 | 2.870 | 0.04229 |
| | | Cohort 3 | 2.071 | 1.210 | 3.860 | 0.01283 |

TABLE 9B
List of 146 combination scores significantly associated
with lack of pCR across the 3 datasets.

| | | | | 95% CI | 95% CI | |
|---|---|---|---|---|---|---|
| | | | Odds | lower | higher | |
| Gene combination | signatures | Dataset | ratio | limit | limit | p-value |
| CXCL8_GRB7 | IGG_HER2 | Cohort 1 | 0.667 | 0.447 | 0.967 | 0.03802 |
| | | Cohort 3 | 0.589 | 0.338 | 0.974 | 0.04697 |
| HLA.C_ERBB2 | IGG_HER2 | Cohort 1 | 0.538 | 0.354 | 0.791 | 0.00234 |
| | | Cohort 2 | 0.575 | 0.326 | 0.951 | 0.04058 |
| | | Cohort 3 | 0.523 | 0.297 | 0.873 | 0.01730 |
| HLA.C_GRB7 | IGG_HER2 | Cohort 1 | 0.502 | 0.328 | 0.742 | 0.00087 |
| | | Cohort 3 | 0.544 | 0.310 | 0.904 | 0.02412 |
| HLA.C_TCAP | IGG_HER2 | Cohort 2 | 0.602 | 0.352 | 0.968 | 0.04539 |
| | | Cohort 3 | 0.537 | 0.309 | 0.892 | 0.02046 |
| NTN3_ERBB2 | IGG_HER2 | Cohort 1 | 0.462 | 0.297 | 0.690 | 0.00030 |
| | | Cohort 3 | 0.422 | 0.219 | 0.740 | 0.00504 |
| NTN3_GRB7 | IGG_HER2 | Cohort 1 | 0.433 | 0.276 | 0.650 | 0.00012 |
| | | Cohort 3 | 0.466 | 0.251 | 0.800 | 0.00932 |
| NTN3_STARD3 | IGG_HER2 | Cohort 1 | 0.478 | 0.306 | 0.714 | 0.00058 |
| | | Cohort 3 | 0.532 | 0.289 | 0.903 | 0.02818 |
| NTN3_TCAP | IGG_HER2 | Cohort 1 | 0.667 | 0.451 | 0.964 | 0.03543 |
| | | Cohort 2 | 0.562 | 0.326 | 0.911 | 0.02597 |
| | | Cohort 3 | 0.445 | 0.237 | 0.764 | 0.00595 |
| CD27_IGL | IGG_IGG | Cohort 1 | 0.662 | 0.444 | 0.962 | 0.03545 |
| | | Cohort 2 | 0.369 | 0.192 | 0.637 | 0.00099 |
| HLA.C_IGKC | IGG_IGG | Cohort 1 | 0.658 | 0.442 | 0.954 | 0.03179 |
| | | Cohort 2 | 0.597 | 0.349 | 0.972 | 0.04592 |
| HLA.C_IGL | IGG_IGG | Cohort 1 | 0.613 | 0.407 | 0.895 | 0.01426 |
| | | Cohort 2 | 0.378 | 0.200 | 0.652 | 0.00112 |
| HLA.C_LAX1 | IGG_IGG | Cohort 1 | 0.673 | 0.452 | 0.975 | 0.04140 |
| | | Cohort 2 | 0.595 | 0.353 | 0.954 | 0.03831 |
| | | Cohort 3 | 0.581 | 0.324 | 0.970 | 0.04877 |
| IGJ_IGL | IGG_IGG | Cohort 1 | 0.648 | 0.433 | 0.942 | 0.02744 |
| | | Cohort 2 | 0.332 | 0.165 | 0.590 | 0.00062 |
| LAX1_IGL | IGG_IGG | Cohort 1 | 0.676 | 0.455 | 0.979 | 0.04370 |
| | | Cohort 2 | 0.445 | 0.243 | 0.749 | 0.00441 |
| NTN3_IGL | IGG_IGG | Cohort 1 | 0.686 | 0.465 | 0.991 | 0.04886 |
| | | Cohort 2 | 0.458 | 0.257 | 0.762 | 0.00433 |
| PIM2_IGL | IGG_IGG | Cohort 1 | 0.625 | 0.414 | 0.913 | 0.01903 |
| | | Cohort 2 | 0.364 | 0.184 | 0.641 | 0.00134 |
| POU2AF1_IGL | IGG_IGG | Cohort 1 | 0.631 | 0.418 | 0.921 | 0.02127 |
| | | Cohort 2 | 0.483 | 0.267 | 0.805 | 0.00889 |
| TNFRSF17_IGL | IGG_IGG | Cohort 1 | 0.658 | 0.441 | 0.956 | 0.03307 |
| | | Cohort 2 | 0.480 | 0.265 | 0.805 | 0.00893 |
| AFF3_ERBB2 | LUM_HER2 | Cohort 1 | 0.446 | 0.285 | 0.668 | 0.00018 |
| | | Cohort 2 | 0.259 | 0.119 | 0.489 | 0.00015 |
| | | Cohort 3 | 0.358 | 0.182 | 0.635 | 0.00112 |
| AFF3_GRB7 | LUM_HER2 | Cohort 1 | 0.424 | 0.269 | 0.639 | 0.00009 |
| | | Cohort 2 | 0.333 | 0.170 | 0.589 | 0.00046 |
| | | Cohort 3 | 0.388 | 0.200 | 0.681 | 0.00217 |
| AFF3_STARD3 | LUM_HER2 | Cohort 1 | 0.434 | 0.275 | 0.654 | 0.00014 |
| | | Cohort 2 | 0.308 | 0.157 | 0.545 | 0.00017 |
| | | Cohort 3 | 0.380 | 0.193 | 0.671 | 0.00199 |
| AFF3_TCAP | LUM_HER2 | Cohort 1 | 0.527 | 0.346 | 0.777 | 0.00182 |
| | | Cohort 2 | 0.219 | 0.098 | 0.420 | 0.00003 |
| | | Cohort 3 | 0.379 | 0.195 | 0.667 | 0.00173 |
| AGR3_ERBB2 | LUM_HER2 | Cohort 1 | 0.440 | 0.281 | 0.659 | 0.00014 |
| | | Cohort 2 | 0.329 | 0.176 | 0.567 | 0.00016 |
| | | Cohort 3 | 0.291 | 0.139 | 0.536 | 0.00027 |
| AGR3_GRB7 | LUM_HER2 | Cohort 1 | 0.420 | 0.266 | 0.632 | 0.00007 |
| | | Cohort 2 | 0.363 | 0.198 | 0.617 | 0.00041 |
| | | Cohort 3 | 0.309 | 0.151 | 0.562 | 0.00039 |
| AGR3_STARD3 | LUM_HER2 | Cohort 1 | 0.451 | 0.290 | 0.673 | 0.00019 |
| | | Cohort 2 | 0.371 | 0.204 | 0.627 | 0.00047 |
| | | Cohort 3 | 0.292 | 0.139 | 0.539 | 0.00031 |
| AGR3_TCAP | LUM_HER2 | Cohort 1 | 0.521 | 0.343 | 0.766 | 0.00137 |
| | | Cohort 2 | 0.320 | 0.168 | 0.554 | 0.00015 |
| | | Cohort 3 | 0.308 | 0.150 | 0.562 | 0.00040 |
| BCL2_ERBB2 | LUM_HER2 | Cohort 1 | 0.397 | 0.250 | 0.602 | 0.00003 |
| | | Cohort 2 | 0.290 | 0.122 | 0.576 | 0.00162 |
| | | Cohort 3 | 0.343 | 0.173 | 0.613 | 0.00080 |
| BCL2_GRB7 | LUM_HER2 | Cohort 1 | 0.376 | 0.235 | 0.572 | 0.00001 |
| | | Cohort 2 | 0.405 | 0.203 | 0.723 | 0.00492 |
| | | Cohort 3 | 0.373 | 0.192 | 0.659 | 0.00156 |
| BCL2_STARD3 | LUM_HER2 | Cohort 1 | 0.361 | 0.220 | 0.560 | 0.00002 |
| | | Cohort 2 | 0.394 | 0.204 | 0.691 | 0.00250 |
| | | Cohort 3 | 0.368 | 0.190 | 0.649 | 0.00124 |
| BCL2_TCAP | LUM_HER2 | Cohort 1 | 0.474 | 0.303 | 0.709 | 0.00053 |
| | | Cohort 2 | 0.281 | 0.130 | 0.527 | 0.00033 |
| | | Cohort 3 | 0.340 | 0.172 | 0.608 | 0.00073 |
| DNAJC12_ERBB2 | LUM_HER2 | Cohort 1 | 0.334 | 0.199 | 0.524 | 0.00001 |
| | | Cohort 2 | 0.347 | 0.161 | 0.649 | 0.00266 |
| | | Cohort 3 | 0.436 | 0.238 | 0.744 | 0.00390 |
| DNAJC12_GRB7 | LUM_HER2 | Cohort 1 | 0.319 | 0.189 | 0.503 | 0.00000 |
| | | Cohort 2 | 0.462 | 0.245 | 0.793 | 0.00913 |
| | | Cohort 3 | 0.454 | 0.250 | 0.772 | 0.00558 |
| DNAJC12_STARD3 | LUM_HER2 | Cohort 1 | 0.318 | 0.185 | 0.505 | 0.00001 |
| | | Cohort 2 | 0.448 | 0.238 | 0.774 | 0.00715 |
| | | Cohort 3 | 0.473 | 0.261 | 0.800 | 0.00798 |
| DNAJC12_TCAP | LUM_HER2 | Cohort 1 | 0.418 | 0.260 | 0.635 | 0.00012 |
| | | Cohort 2 | 0.332 | 0.160 | 0.607 | 0.00105 |
| | | Cohort 3 | 0.440 | 0.238 | 0.754 | 0.00488 |
| ESR1_ERBB2 | LUM_HER2 | Cohort 1 | 0.406 | 0.259 | 0.610 | 0.00003 |
| | | Cohort 2 | 0.225 | 0.096 | 0.440 | 0.00010 |
| | | Cohort 3 | 0.326 | 0.166 | 0.579 | 0.00037 |
| ESR1_GRB7 | LUM_HER2 | Cohort 1 | 0.392 | 0.248 | 0.591 | 0.00002 |
| | | Cohort 2 | 0.290 | 0.140 | 0.528 | 0.00021 |
| | | Cohort 3 | 0.345 | 0.177 | 0.609 | 0.00062 |
| ESR1_STARD3 | LUM_HER2 | Cohort 1 | 0.398 | 0.253 | 0.600 | 0.00003 |
| | | Cohort 2 | 0.253 | 0.118 | 0.472 | 0.00008 |
| | | Cohort 3 | 0.334 | 0.171 | 0.592 | 0.00046 |
| ESR1_TCAP | LUM_HER2 | Cohort 1 | 0.466 | 0.304 | 0.692 | 0.00026 |
| | | Cohort 2 | 0.189 | 0.074 | 0.389 | 0.00006 |
| | | Cohort 3 | 0.334 | 0.171 | 0.593 | 0.00047 |
| AFF3_CD27 | LUM_IGG | Cohort 1 | 0.505 | 0.328 | 0.747 | 0.00105 |
| | | Cohort 2 | 0.255 | 0.111 | 0.498 | 0.00031 |
| | | Cohort 3 | 0.419 | 0.222 | 0.726 | 0.00358 |
| AFF3_CD79A | LUM_IGG | Cohort 1 | 0.518 | 0.337 | 0.767 | 0.00162 |
| | | Cohort 2 | 0.295 | 0.140 | 0.545 | 0.00037 |
| | | Cohort 3 | 0.467 | 0.254 | 0.798 | 0.00848 |
| AFF3_CXCL8 | LUM_IGG | Cohort 1 | 0.583 | 0.387 | 0.852 | 0.00697 |
| | | Cohort 2 | 0.271 | 0.128 | 0.502 | 0.00015 |
| | | Cohort 3 | 0.493 | 0.271 | 0.835 | 0.01271 |
| AFF3_HLA.C | LUM_IGG | Cohort 1 | 0.586 | 0.392 | 0.854 | 0.00688 |
| | | Cohort 2 | 0.301 | 0.144 | 0.550 | 0.00038 |
| | | Cohort 3 | 0.504 | 0.278 | 0.851 | 0.01516 |
| AFF3_IGJ | LUM_IGG | Cohort 1 | 0.558 | 0.368 | 0.819 | 0.00402 |
| | | Cohort 2 | 0.302 | 0.140 | 0.568 | 0.00070 |
| | | Cohort 3 | 0.456 | 0.234 | 0.805 | 0.01223 |
| AFF3_IGKC | LUM_IGG | Cohort 1 | 0.520 | 0.337 | 0.771 | 0.00186 |
| | | Cohort 2 | 0.288 | 0.134 | 0.538 | 0.00037 |
| | | Cohort 3 | 0.476 | 0.247 | 0.835 | 0.01639 |
| AFF3_IGL | LUM_IGG | Cohort 1 | 0.497 | 0.320 | 0.740 | 0.00098 |
| | | Cohort 2 | 0.215 | 0.092 | 0.421 | 0.00006 |
| | | Cohort 3 | 0.478 | 0.258 | 0.818 | 0.01130 |
| AFF3_IGLV3.25 | LUM_IGG | Cohort 1 | 0.561 | 0.370 | 0.822 | 0.00422 |
| | | Cohort 2 | 0.336 | 0.170 | 0.591 | 0.00050 |
| | | Cohort 3 | 0.542 | 0.304 | 0.906 | 0.02589 |
| AFF3_IL2RG | LUM_IGG | Cohort 1 | 0.486 | 0.311 | 0.726 | 0.00079 |
| | | Cohort 2 | 0.315 | 0.151 | 0.579 | 0.00066 |
| | | Cohort 3 | 0.435 | 0.229 | 0.755 | 0.00569 |
| AFF3_LAX1 | LUM_IGG | Cohort 1 | 0.515 | 0.335 | 0.762 | 0.00145 |
| | | Cohort 2 | 0.235 | 0.101 | 0.462 | 0.00016 |
| | | Cohort 3 | 0.438 | 0.233 | 0.757 | 0.00562 |
| AFF3_NTN3 | LUM_IGG | Cohort 1 | 0.594 | 0.397 | 0.865 | 0.00822 |
| | | Cohort 2 | 0.312 | 0.154 | 0.564 | 0.00037 |
| | | Cohort 3 | 0.496 | 0.273 | 0.837 | 0.01287 |
| AFF3_PIM2 | LUM_IGG | Cohort 1 | 0.524 | 0.344 | 0.772 | 0.00162 |
| | | Cohort 2 | 0.258 | 0.119 | 0.485 | 0.00013 |
| | | Cohort 3 | 0.473 | 0.260 | 0.802 | 0.00836 |
| AFF3_POU2AF1 | LUM_IGG | Cohort 1 | 0.550 | 0.360 | 0.809 | 0.00347 |
| | | Cohort 2 | 0.232 | 0.100 | 0.455 | 0.00013 |
| | | Cohort 3 | 0.468 | 0.256 | 0.796 | 0.00814 |
| AFF3_TNFRSF17 | LUM_IGG | Cohort 1 | 0.519 | 0.338 | 0.766 | 0.00156 |
| | | Cohort 2 | 0.221 | 0.094 | 0.437 | 0.00010 |
| | | Cohort 3 | 0.489 | 0.269 | 0.828 | 0.01170 |
| AGR3_CD27 | LUM_IGG | Cohort 1 | 0.534 | 0.353 | 0.783 | 0.00192 |
| | | Cohort 2 | 0.406 | 0.228 | 0.679 | 0.00110 |
| | | Cohort 3 | 0.328 | 0.168 | 0.584 | 0.00041 |
| AGR3_CD79A | LUM_IGG | Cohort 1 | 0.518 | 0.338 | 0.766 | 0.00150 |
| | | Cohort 2 | 0.399 | 0.221 | 0.672 | 0.00110 |
| | | Cohort 3 | 0.370 | 0.195 | 0.647 | 0.00104 |
| AGR3_CXCL8 | LUM_IGG | Cohort 1 | 0.577 | 0.386 | 0.843 | 0.00561 |
| | | Cohort 2 | 0.355 | 0.194 | 0.602 | 0.00030 |
| | | Cohort 3 | 0.382 | 0.203 | 0.663 | 0.00129 |
| AGR3_HLA.C | LUM_IGG | Cohort 1 | 0.575 | 0.385 | 0.838 | 0.00499 |
| | | Cohort 2 | 0.407 | 0.227 | 0.682 | 0.00122 |
| | | Cohort 3 | 0.363 | 0.191 | 0.635 | 0.00086 |
| AGR3_IGJ | LUM_IGG | Cohort 1 | 0.550 | 0.363 | 0.807 | 0.00315 |
| | | Cohort 2 | 0.408 | 0.224 | 0.691 | 0.00164 |
| | | Cohort 3 | 0.339 | 0.172 | 0.610 | 0.00074 |
| AGR3_IGKC | LUM_IGG | Cohort 1 | 0.516 | 0.337 | 0.763 | 0.00143 |
| | | Cohort 2 | 0.379 | 0.205 | 0.648 | 0.00085 |
| | | Cohort 3 | 0.356 | 0.181 | 0.638 | 0.00121 |
| AGR3_IGL | LUM_IGG | Cohort 1 | 0.489 | 0.315 | 0.729 | 0.00076 |
| | | Cohort 2 | 0.301 | 0.154 | 0.533 | 0.00013 |
| | | Cohort 3 | 0.355 | 0.183 | 0.629 | 0.00092 |
| AGR3_IGLV3.25 | LUM_IGG | Cohort 1 | 0.540 | 0.355 | 0.795 | 0.00255 |
| | | Cohort 2 | 0.403 | 0.223 | 0.671 | 0.00103 |
| | | Cohort 3 | 0.398 | 0.210 | 0.693 | 0.00222 |
| AGR3_IL2RG | LUM_IGG | Cohort 1 | 0.512 | 0.334 | 0.756 | 0.00121 |
| | | Cohort 2 | 0.437 | 0.250 | 0.724 | 0.00211 |
| | | Cohort 3 | 0.318 | 0.159 | 0.572 | 0.00038 |
| AGR3_LAX1 | LUM_IGG | Cohort 1 | 0.529 | 0.349 | 0.777 | 0.00171 |
| | | Cohort 2 | 0.366 | 0.200 | 0.619 | 0.00041 |
| | | Cohort 3 | 0.334 | 0.171 | 0.593 | 0.00047 |
| AGR3_NTN3 | LUM_IGG | Cohort 1 | 0.588 | 0.396 | 0.854 | 0.00647 |
| | | Cohort 2 | 0.399 | 0.223 | 0.670 | 0.00095 |
| | | Cohort 3 | 0.372 | 0.195 | 0.649 | 0.00110 |
| AGR3_PIM2 | LUM_IGG | Cohort 1 | 0.537 | 0.355 | 0.789 | 0.00219 |
| | | Cohort 2 | 0.372 | 0.205 | 0.627 | 0.00045 |
| | | Cohort 3 | 0.356 | 0.186 | 0.625 | 0.00075 |
| AGR3_POU2AF1 | LUM_IGG | Cohort 1 | 0.553 | 0.366 | 0.810 | 0.00328 |
| | | Cohort 2 | 0.357 | 0.193 | 0.609 | 0.00038 |
| | | Cohort 3 | 0.371 | 0.196 | 0.647 | 0.00102 |
| AGR3_TNFRSF17 | LUM_IGG | Cohort 1 | 0.532 | 0.352 | 0.782 | 0.00187 |
| | | Cohort 2 | 0.353 | 0.191 | 0.602 | 0.00032 |
| | | Cohort 3 | 0.375 | 0.199 | 0.651 | 0.00103 |
| BCL2_CD27 | LUM_IGG | Cohort 1 | 0.361 | 0.216 | 0.568 | 0.00003 |
| | | Cohort 2 | 0.281 | 0.124 | 0.556 | 0.00089 |
| | | Cohort 3 | 0.303 | 0.140 | 0.566 | 0.00070 |
| BCL2_CD79A | LUM_IGG | Cohort 1 | 0.452 | 0.288 | 0.680 | 0.00027 |
| | | Cohort 2 | 0.400 | 0.215 | 0.684 | 0.00171 |
| | | Cohort 3 | 0.417 | 0.219 | 0.725 | 0.00375 |
| BCL2_CXCL8 | LUM_IGG | Cohort 1 | 0.533 | 0.345 | 0.788 | 0.00256 |
| | | Cohort 2 | 0.320 | 0.161 | 0.571 | 0.00037 |
| | | Cohort 3 | 0.479 | 0.254 | 0.823 | 0.01293 |
| BCL2_HLA.C | LUM_IGG | Cohort 1 | 0.497 | 0.324 | 0.735 | 0.00075 |
| | | Cohort 2 | 0.339 | 0.152 | 0.641 | 0.00290 |
| | | Cohort 3 | 0.465 | 0.248 | 0.802 | 0.00990 |
| BCL2_IGJ | LUM_IGG | Cohort 1 | 0.464 | 0.293 | 0.700 | 0.00051 |
| | | Cohort 2 | 0.433 | 0.227 | 0.755 | 0.00597 |
| | | Cohort 3 | 0.441 | 0.215 | 0.790 | 0.01258 |
| BCL2_IGKC | LUM_IGG | Cohort 1 | 0.451 | 0.285 | 0.681 | 0.00031 |
| | | Cohort 2 | 0.357 | 0.182 | 0.639 | 0.00124 |
| | | Cohort 3 | 0.452 | 0.228 | 0.802 | 0.01247 |
| BCL2_IGL | LUM_IGG | Cohort 1 | 0.442 | 0.279 | 0.668 | 0.00023 |
| | | Cohort 2 | 0.255 | 0.117 | 0.479 | 0.00012 |
| | | Cohort 3 | 0.451 | 0.238 | 0.780 | 0.00781 |
| BCL2_IGLV3.25 | LUM_IGG | Cohort 1 | 0.544 | 0.359 | 0.800 | 0.00281 |
| | | Cohort 2 | 0.456 | 0.259 | 0.749 | 0.00335 |
| | | Cohort 3 | 0.548 | 0.307 | 0.915 | 0.02842 |
| BCL2_IL2RG | LUM_IGG | Cohort 1 | 0.365 | 0.217 | 0.575 | 0.00004 |
| | | Cohort 2 | 0.381 | 0.184 | 0.696 | 0.00423 |
| | | Cohort 3 | 0.386 | 0.199 | 0.679 | 0.00210 |
| BCL2_LAX1 | LUM_IGG | Cohort 1 | 0.396 | 0.243 | 0.611 | 0.00008 |
| | | Cohort 2 | 0.265 | 0.123 | 0.500 | 0.00017 |
| | | Cohort 3 | 0.353 | 0.171 | 0.639 | 0.00169 |
| BCL2_NTN3 | LUM_IGG | Cohort 1 | 0.501 | 0.326 | 0.743 | 0.00096 |
| | | Cohort 2 | 0.327 | 0.163 | 0.591 | 0.00061 |
| | | Cohort 3 | 0.442 | 0.230 | 0.768 | 0.00718 |
| BCL2_PIM2 | LUM_IGG | Cohort 1 | 0.426 | 0.271 | 0.642 | 0.00010 |
| | | Cohort 2 | 0.311 | 0.158 | 0.552 | 0.00022 |
| | | Cohort 3 | 0.408 | 0.212 | 0.711 | 0.00324 |
| BCL2_POU2AF1 | LUM_IGG | Cohort 1 | 0.470 | 0.300 | 0.706 | 0.00051 |
| | | Cohort 2 | 0.295 | 0.147 | 0.531 | 0.00017 |
| | | Cohort 3 | 0.406 | 0.212 | 0.710 | 0.00316 |
| BCL2_TNFRSF17 | LUM_IGG | Cohort 1 | 0.411 | 0.255 | 0.627 | 0.00009 |
| | | Cohort 2 | 0.285 | 0.141 | 0.517 | 0.00014 |
| | | Cohort 3 | 0.428 | 0.220 | 0.750 | 0.00608 |
| DNAJC12_CD27 | LUM_IGG | Cohort 1 | 0.383 | 0.232 | 0.594 | 0.00006 |
| | | Cohort 2 | 0.511 | 0.277 | 0.866 | 0.01974 |
| | | Cohort 3 | 0.498 | 0.274 | 0.842 | 0.01386 |
| DNAJC12_CD79A | LUM_IGG | Cohort 1 | 0.437 | 0.276 | 0.661 | 0.00019 |
| | | Cohort 2 | 0.503 | 0.282 | 0.837 | 0.01231 |
| | | Cohort 3 | 0.548 | 0.309 | 0.915 | 0.02819 |
| DNAJC12_CXCL8 | LUM_IGG | Cohort 1 | 0.465 | 0.294 | 0.701 | 0.00051 |
| | | Cohort 2 | 0.306 | 0.138 | 0.586 | 0.00120 |
| | | Cohort 3 | 0.580 | 0.333 | 0.961 | 0.04178 |
| DNAJC12_HLA.C | LUM_IGG | Cohort 1 | 0.435 | 0.272 | 0.658 | 0.00019 |
| | | Cohort 2 | 0.518 | 0.279 | 0.880 | 0.02339 |
| DNAJC12_IGJ | LUM_IGG | Cohort 1 | 0.422 | 0.264 | 0.640 | 0.00012 |
| | | Cohort 2 | 0.542 | 0.302 | 0.904 | 0.02674 |
| | | Cohort 3 | 0.563 | 0.310 | 0.948 | 0.04151 |
| DNAJC12_IGKC | LUM_IGG | Cohort 1 | 0.417 | 0.257 | 0.639 | 0.00015 |
| | | Cohort 2 | 0.454 | 0.244 | 0.776 | 0.00697 |
| | | Cohort 3 | 0.559 | 0.306 | 0.944 | 0.04102 |
| DNAJC12_IGL | LUM_IGG | Cohort 1 | 0.409 | 0.251 | 0.628 | 0.00012 |
| | | Cohort 2 | 0.335 | 0.168 | 0.597 | 0.00064 |
| | | Cohort 3 | 0.548 | 0.306 | 0.918 | 0.02987 |
| DNAJC12_IGLV3.25 | LUM_IGG | Cohort 1 | 0.487 | 0.314 | 0.725 | 0.00067 |
| | | Cohort 2 | 0.499 | 0.288 | 0.814 | 0.00793 |
| DNAJC12_IL2RG | LUM_IGG | Cohort 1 | 0.382 | 0.230 | 0.595 | 0.00007 |
| | | Cohort 2 | 0.544 | 0.298 | 0.913 | 0.03102 |
| | | Cohort 3 | 0.526 | 0.290 | 0.887 | 0.02250 |
| DNAJC12_LAX1 | LUM_IGG | Cohort 1 | 0.387 | 0.236 | 0.600 | 0.00006 |
| | | Cohort 2 | 0.410 | 0.212 | 0.717 | 0.00371 |
| | | Cohort 3 | 0.521 | 0.286 | 0.880 | 0.02124 |
| DNAJC12_NTN3 | LUM_IGG | Cohort 1 | 0.423 | 0.263 | 0.644 | 0.00015 |
| | | Cohort 2 | 0.476 | 0.248 | 0.825 | 0.01468 |
| DNAJC12_PIM2 | LUM_IGG | Cohort 1 | 0.408 | 0.254 | 0.621 | 0.00008 |
| | | Cohort 2 | 0.438 | 0.236 | 0.745 | 0.00442 |
| | | Cohort 3 | 0.558 | 0.318 | 0.928 | 0.03101 |
| DNAJC12_POU2AF1 | LUM_IGG | Cohort 1 | 0.445 | 0.280 | 0.673 | 0.00027 |
| | | Cohort 2 | 0.421 | 0.226 | 0.720 | 0.00312 |
| | | Cohort 3 | 0.545 | 0.307 | 0.910 | 0.02661 |
| DNAJC12_TNFRSF17 | LUM_IGG | Cohort 1 | 0.389 | 0.237 | 0.600 | 0.00006 |
| | | Cohort 2 | 0.407 | 0.216 | 0.700 | 0.00241 |
| | | Cohort 3 | 0.580 | 0.330 | 0.963 | 0.04356 |
| ESR1_CD27 | LUM_IGG | Cohort 1 | 0.420 | 0.268 | 0.632 | 0.00007 |
| | | Cohort 2 | 0.243 | 0.104 | 0.474 | 0.00020 |
| | | Cohort 3 | 0.345 | 0.180 | 0.608 | 0.00055 |
| ESR1_CD79A | LUM_IGG | Cohort 1 | 0.423 | 0.266 | 0.640 | 0.00011 |
| | | Cohort 2 | 0.285 | 0.138 | 0.520 | 0.00017 |
| | | Cohort 3 | 0.385 | 0.205 | 0.668 | 0.00139 |
| ESR1_CXCL8 | LUM_IGG | Cohort 1 | 0.504 | 0.331 | 0.742 | 0.00082 |
| | | Cohort 2 | 0.241 | 0.106 | 0.463 | 0.00012 |
| | | Cohort 3 | 0.413 | 0.225 | 0.707 | 0.00224 |
| ESR1_HLA.C | LUM_IGG | Cohort 1 | 0.467 | 0.305 | 0.691 | 0.00024 |
| | | Cohort 2 | 0.236 | 0.102 | 0.460 | 0.00014 |
| | | Cohort 3 | 0.399 | 0.217 | 0.684 | 0.00154 |
| ESR1_IGJ | LUM_IGG | Cohort 1 | 0.444 | 0.283 | 0.667 | 0.00018 |
| | | Cohort 2 | 0.298 | 0.144 | 0.549 | 0.00035 |
| | | Cohort 3 | 0.368 | 0.186 | 0.657 | 0.00167 |
| ESRI_IGKC | LUM_IGG | Cohort 1 | 0.418 | 0.260 | 0.636 | 0.00011 |
| | | Cohort 2 | 0.265 | 0.123 | 0.497 | 0.00016 |
| | | Cohort 3 | 0.391 | 0.203 | 0.691 | 0.00248 |
| ESR1_IGL | LUM_IGG | Cohort 1 | 0.412 | 0.257 | 0.627 | 0.00009 |
| | | Cohort 2 | 0.193 | 0.080 | 0.388 | 0.00004 |
| | | Cohort 3 | 0.383 | 0.201 | 0.669 | 0.00158 |
| ESR1_IGLV3.25 | LUM_IGG | Cohort 1 | 0.468 | 0.300 | 0.699 | 0.00039 |
| | | Cohort 2 | 0.303 | 0.148 | 0.544 | 0.00026 |
| | | Cohort 3 | 0.445 | 0.245 | 0.755 | 0.00432 |
| ESR1_IL2RG | LUM_IGG | Cohort 1 | 0.407 | 0.256 | 0.617 | 0.00006 |
| | | Cohort 2 | 0.297 | 0.143 | 0.545 | 0.00032 |
| | | Cohort 3 | 0.344 | 0.176 | 0.609 | 0.00065 |
| ESR1_LAX1 | LUM_IGG | Cohort 1 | 0.427 | 0.272 | 0.642 | 0.00009 |
| | | Cohort 2 | 0.217 | 0.089 | 0.432 | 0.00011 |
| | | Cohort 3 | 0.359 | 0.188 | 0.629 | 0.00079 |
| ESR1_NTN3 | LUM_IGG | Cohort 1 | 0.484 | 0.319 | 0.713 | 0.00039 |
| | | Cohort 2 | 0.263 | 0.118 | 0.498 | 0.00021 |
| | | Cohort 3 | 0.401 | 0.217 | 0.689 | 0.00175 |
| ESR1_PIM2 | LUM_IGG | Cohort 1 | 0.429 | 0.274 | 0.643 | 0.00009 |
| | | Cohort 2 | 0.230 | 0.102 | 0.442 | 0.00007 |
| | | Cohort 3 | 0.380 | 0.203 | 0.658 | 0.00112 |
| ESR1_POU2AF1 | LUM_IGG | Cohort 1 | 0.450 | 0.289 | 0.674 | 0.00021 |
| | | Cohort 2 | 0.223 | 0.096 | 0.434 | 0.00007 |
| | | Cohort 3 | 0.391 | 0.210 | 0.674 | 0.00142 |
| ESR1_TNFRSF17 | LUM_IGG | Cohort 1 | 0.428 | 0.273 | 0.642 | 0.00009 |
| | | Cohort 2 | 0.212 | 0.088 | 0.421 | 0.00008 |
| | | Cohort 3 | 0.401 | 0.217 | 0.690 | 0.00178 |
| AGR3_BCL2 | LUM_LUM | Cohort 2 | 0.549 | 0.322 | 0.896 | 0.02058 |
| | | Cohort 3 | 0.444 | 0.243 | 0.756 | 0.00454 |
| AGR3_DNAJC12 | LUM_LUM | Cohort 2 | 0.552 | 0.324 | 0.897 | 0.02080 |
| | | Cohort 3 | 0.388 | 0.201 | 0.680 | 0.00202 |
| ESR1_AFF3 | LUM_LUM | Cohort 1 | 0.598 | 0.393 | 0.876 | 0.01135 |
| | | Cohort 3 | 0.577 | 0.326 | 0.961 | 0.04385 |
| ESR1_BCL2 | LUM_LUM | Cohort 1 | 0.575 | 0.385 | 0.839 | 0.00520 |
| | | Cohort 2 | 0.342 | 0.164 | 0.624 | 0.00142 |
| | | Cohort 3 | 0.471 | 0.265 | 0.792 | 0.00659 |
| ESR1_DNAJC12 | LUM_LUM | Cohort 2 | 0.444 | 0.236 | 0.762 | 0.00603 |
| | | Cohort 3 | 0.437 | 0.233 | 0.753 | 0.00519 |
| AFF3_ASPM | LUM_PROLIF | Cohort 1 | 0.466 | 0.298 | 0.697 | 0.00039 |
| | | Cohort 2 | 0.276 | 0.130 | 0.514 | 0.00020 |
| | | Cohort 3 | 0.429 | 0.228 | 0.741 | 0.00442 |
| AFF3_EXO1 | LUM_PROLIF | Cohort 1 | 0.498 | 0.324 | 0.736 | 0.00079 |
| | | Cohort 2 | 0.325 | 0.164 | 0.580 | 0.00043 |
| | | Cohort 3 | 0.408 | 0.213 | 0.710 | 0.00306 |
| AFF3_KIF23 | LUM_PROLIF | Cohort 1 | 0.475 | 0.306 | 0.708 | 0.00046 |
| | | Cohort 2 | 0.299 | 0.149 | 0.538 | 0.00019 |
| | | Cohort 3 | 0.455 | 0.246 | 0.778 | 0.00663 |
| AFF3_NEK2 | LUM_PROLIF | Cohort 1 | 0.504 | 0.329 | 0.746 | 0.00099 |
| | | Cohort 2 | 0.321 | 0.162 | 0.572 | 0.00036 |
| | | Cohort 3 | 0.483 | 0.264 | 0.819 | 0.01072 |
| AGR3_ASPM | LUM_PROLIF | Cohort 1 | 0.491 | 0.321 | 0.726 | 0.00059 |
| | | Cohort 2 | 0.385 | 0.216 | 0.644 | 0.00056 |
| | | Cohort 3 | 0.289 | 0.135 | 0.539 | 0.00037 |
| AGR3_EXO1 | LUM_PROLIF | Cohort 1 | 0.516 | 0.341 | 0.758 | 0.00109 |
| | | Cohort 2 | 0.415 | 0.236 | 0.688 | 0.00114 |
| | | Cohort 3 | 0.278 | 0.129 | 0.521 | 0.00027 |
| AGR3_KIF23 | LUM_PROLIF | Cohort 1 | 0.505 | 0.333 | 0.744 | 0.00084 |
| | | Cohort 2 | 0.385 | 0.217 | 0.644 | 0.00053 |
| | | Cohort 3 | 0.317 | 0.156 | 0.574 | 0.00046 |
| AGR3_NEK2 | LUM_PROLIF | Cohort 1 | 0.518 | 0.342 | 0.761 | 0.00116 |
| | | Cohort 2 | 0.400 | 0.225 | 0.666 | 0.00082 |
| | | Cohort 3 | 0.313 | 0.151 | 0.571 | 0.00050 |
| BCL2_ASPM | LUM_PROLIF | Cohort 1 | 0.374 | 0.227 | 0.580 | 0.00003 |
| | | Cohort 2 | 0.297 | 0.135 | 0.570 | 0.00087 |
| | | Cohort 3 | 0.417 | 0.220 | 0.723 | 0.00358 |
| BCL2_EXO1 | LUM_PROLIF | Cohort 1 | 0.407 | 0.254 | 0.618 | 0.00007 |
| | | Cohort 2 | 0.426 | 0.224 | 0.740 | 0.00481 |
| | | Cohort 3 | 0.358 | 0.182 | 0.638 | 0.00120 |
| BCL2_KIF23 | LUM_PROLIF | Cohort 1 | 0.365 | 0.220 | 0.568 | 0.00003 |
| | | Cohort 2 | 0.332 | 0.162 | 0.606 | 0.00096 |
| | | Cohort 3 | 0.435 | 0.233 | 0.749 | 0.00476 |
| BCL2_NEK2 | LUM_PROLIF | Cohort 1 | 0.413 | 0.256 | 0.630 | 0.00010 |
| | | Cohort 2 | 0.384 | 0.195 | 0.683 | 0.00257 |
| | | Cohort 3 | 0.514 | 0.284 | 0.866 | 0.01777 |
| DNAJC12_ASPM | LUM_PROLIF | Cohort 1 | 0.344 | 0.204 | 0.541 | 0.00002 |
| | | Cohort 2 | 0.426 | 0.207 | 0.774 | 0.01088 |
| | | Cohort 3 | 0.517 | 0.288 | 0.869 | 0.01792 |
| DNAJC12_EXO1 | LUM_PROLIF | Cohort 1 | 0.359 | 0.216 | 0.559 | 0.00002 |
| | | Cohort 2 | 0.511 | 0.268 | 0.879 | 0.02520 |
| | | Cohort 3 | 0.481 | 0.262 | 0.818 | 0.01072 |
| DNAJC12_KIF23 | LUM_PROLIF | Cohort 1 | 0.341 | 0.201 | 0.538 | 0.00002 |
| | | Cohort 2 | 0.463 | 0.239 | 0.810 | 0.01279 |
| | | Cohort 3 | 0.554 | 0.317 | 0.921 | 0.02861 |
| DNAJC12_NEK2 | LUM_PROLIF | Cohort 1 | 0.361 | 0.216 | 0.564 | 0.00003 |
| | | Cohort 2 | 0.499 | 0.262 | 0.859 | 0.02033 |
| ESR1_ASPM | LUM_PROLIF | Cohort 1 | 0.410 | 0.261 | 0.617 | 0.00004 |
| | | Cohort 2 | 0.197 | 0.076 | 0.409 | 0.00011 |
| | | Cohort 3 | 0.310 | 0.154 | 0.561 | 0.00034 |
| ESR1_EXO1 | LUM_PROLIF | Cohort 1 | 0.426 | 0.274 | 0.637 | 0.00007 |
| | | Cohort 2 | 0.256 | 0.114 | 0.490 | 0.00021 |
| | | Cohort 3 | 0.296 | 0.146 | 0.540 | 0.00023 |
| ESR1_KIF23 | LUM_PROLIF | Cohort 1 | 0.413 | 0.264 | 0.621 | 0.00005 |
| | | Cohort 2 | 0.218 | 0.090 | 0.435 | 0.00011 |
| | | Cohort 3 | 0.338 | 0.174 | 0.599 | 0.00052 |
| ESR1_NEK2 | LUM_PROLIF | Cohort 1 | 0.422 | 0.270 | 0.634 | 0.00007 |
| | | Cohort 2 | 0.219 | 0.091 | 0.438 | 0.00012 |
| | | Cohort 3 | 0.335 | 0.170 | 0.598 | 0.00057 |
| ASPM_ERBB2 | PROLIF_HER2 | Cohort 1 | 0.663 | 0.445 | 0.962 | 0.03519 |
| | | Cohort 3 | 0.558 | 0.318 | 0.928 | 0.03096 |
| ASPM_GRB7 | PROLIF_HER2 | Cohort 1 | 0.607 | 0.403 | 0.886 | 0.01241 |
| | | Cohort 3 | 0.584 | 0.336 | 0.966 | 0.04338 |
| EXO1_ERBB2 | PROLIF_HER2 | Cohort 1 | 0.626 | 0.419 | 0.909 | 0.01676 |
| | | Cohort 2 | 0.515 | 0.276 | 0.876 | 0.02271 |
| | | Cohort 3 | 0.595 | 0.346 | 0.981 | 0.04878 |
| EXO1_TCAP | PROLIF_HER2 | Cohort 2 | 0.512 | 0.289 | 0.844 | 0.01328 |
| | | Cohort 3 | 0.599 | 0.351 | 0.985 | 0.04950 |
| KIF23_ERBB2 | PROLIF_HER2 | Cohort 1 | 0.615 | 0.410 | 0.895 | 0.01399 |
| | | Cohort 2 | 0.534 | 0.284 | 0.912 | 0.03332 |
| | | Cohort 3 | 0.481 | 0.268 | 0.810 | 0.00867 |
| KIF23_GRB7 | PROLIF_HER2 | Cohort 1 | 0.561 | 0.371 | 0.823 | 0.00428 |
| | | Cohort 3 | 0.512 | 0.288 | 0.858 | 0.01525 |
| KIF23_STARD3 | PROLIF_HER2 | Cohort 1 | 0.679 | 0.461 | 0.980 | 0.04271 |
| | | Cohort 3 | 0.562 | 0.320 | 0.934 | 0.03285 |
| KIF23_TCAP | PROLIF_HER2 | Cohort 2 | 0.551 | 0.316 | 0.895 | 0.02283 |
| | | Cohort 3 | 0.501 | 0.281 | 0.841 | 0.01242 |
| NEK2_ERBB2 | PROLIF_HER2 | Cohort 1 | 0.613 | 0.411 | 0.890 | 0.01235 |
| | | Cohort 2 | 0.507 | 0.264 | 0.875 | 0.02449 |
| | | Cohort 3 | 0.459 | 0.252 | 0.780 | 0.00638 |
| NEK2_GRB7 | PROLIF_HER2 | Cohort 1 | 0.565 | 0.375 | 0.826 | 0.00434 |
| | | Cohort 3 | 0.488 | 0.272 | 0.822 | 0.01010 |
| NEK2_STARD3 | PROLIF_HER2 | Cohort 1 | 0.669 | 0.454 | 0.967 | 0.03629 |
| | | Cohort 3 | 0.527 | 0.293 | 0.885 | 0.02135 |
| NEK2_TCAP | PROLIF_HER2 | Cohort 2 | 0.522 | 0.291 | 0.862 | 0.01771 |
| | | Cohort 3 | 0.483 | 0.270 | 0.813 | 0.00896 |
| NEK2_ASPM | PROLIF_PROLIF | Cohort 1 | 0.660 | 0.435 | 0.965 | 0.03967 |
| | | Cohort 2 | 0.594 | 0.348 | 0.964 | 0.04229 |
| | | Cohort 3 | 0.483 | 0.259 | 0.826 | 0.01283 |

The combination scores predictive of pCR represent different combinations of the 4 signatures (i.e., immune-luminal, proliferation-luminal, HER2-immune, HER2-proliferation, HER2-luminal, immune-immune, luminal-luminal, proliferation-proliferation). Specifically, 48% (n=70) are pairs composed of genes coming from the immune-luminal signatures, 14% (n=20) are pairs composed of genes from the proliferation-luminal signatures, 6% (n=8) are pairs composed of genes from the HER2-immune signatures, 8% (n=12) are pairs composed of genes from the HER2-proliferation signatures, and 14% (n=20) are pairs composed of genes from the HER2-luminal signatures (Table 10). The combination scores predictive of lack of pCR represent different combinations of the 4 signatures (i.e., luminal-immune, luminal-proliferation, immune-HER2, proliferation-HER2, luminal-HER2, immune-immune, luminal-luminal and proliferation-proliferation). Specifically, 48% (n=70) are pairs composed of genes coming from the luminal-immune signatures, 14% (n=20) are pairs composed of genes from the luminal-proliferation signatures, 6% (n=8) are pairs composed of genes from the immune-HER2 signatures, 8% (n=12) are pairs composed of genes from the proliferation-HER2 signatures, and 14% (n=20) are pairs composed of genes from the luminal-HER2 signatures (Table 10).

TABLE 10
Number of significant combination scores from each signature

Gene 2

| | | IGG | LUM | PROLIF | HER2 |
|---|---|---|---|---|---|
| Gene | IGG | 10 | 70 | 0 | 8 |
| 1 | LUM | 70 | 5 | 20 | 20 |
| | PROLIF | 0 | 20 | 1 | 12 |
| | HER2 | 8 | 20 | 12 | 0 |

*IGG: Immune signature, LUM: luminal signature, PROLIF: proliferation signature, HER2: HER2 amplicon

1. In vitro method for identifying biomarker signatures for the prognosis of patients suffering from HER2+ breast cancer, which comprises:
a. Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1], in a biological sample obtained from the patient;
b. Determining a combination score value by calculating the ratio of the expression of the 2 genes; and
c. Wherein if a deviation of the combination score value is identified, as compared with a pre-established reference value, this is indicative that the biomarker signature may be used for the prognosis of patients suffering from HER2+ breast cancer.

2. In vitro method for the prognosis of patients suffering from HER2+ breast cancer, according to claim 1, which comprises:
a. Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1], in a biological sample obtained from the patient;
b. Determining a combination score value by calculating the ratio of the expression of the 2 genes; and
c. Wherein if a deviation of the combination score value is identified, as compared with a pre-established reference value, this is indicative of the prognosis of patients suffering from HER2+ breast cancer.

3. In vitro method for the prognosis of patients suffering from HER2+ breast cancer, according to claim 1, which comprises:
a. Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1], in a biological sample obtained from the patient;
b. Determining a combination score value by calculating the ratio of the expression of the 2 genes, wherein the ratio is calculated by:
i. Combining a first gene comprised in the immune signature with a second gene comprised in the tumor cell proliferation signature; or
ii. Combining a first gene comprised in the immune signature with a second gene comprised in the luminal differentiation signature; or
iii. Combining a first gene comprised in the luminal differentiation signature with a second gene comprised in the tumor cell proliferation signature; or
iv. Combining a first gene comprised in the immune signature selected from the group consisting of: CD79A, CD27, IGJ, POU2AF1, TNFRSF17, IL2RG, PIM2 or IGL with a second gene comprised in the immune signature selected from the group consisting of: CD27, CXCL8, HLA-C, IGLV3-25, IL2RG, LAX1, NTN3, PIM2 or POU2AF1;
c. Wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EX01, ASPM, NEK2 or KIF23] and the luminal differentiation signature comprises the genes: [BCL2, DNAJC12, AGR3, AFF3 or ESR1]; and
d. Wherein if a deviation of the combination score value is identified, as compared with a pre-established reference value, is indicative of good prognosis.

4. In vitro method for the prognosis of patients suffering from HER2+ breast cancer, according to at claim 1, which comprises:
a. Measuring the level of expression of at least two genes selected from the gene combinations of Table 7A, in a biological sample obtained from the patient;
b. Determining a combination score value by calculating the ratio of the expression of the 2 genes; and
c. Wherein if a deviation of the combination score value is identified, as compared with a pre-established reference value, is indicative of good prognosis.

5. In vitro method for the prognosis of patients suffering from HER2+ breast cancer, according to claim 1, which comprises:
a. Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1], in a biological sample obtained from the patient;
b. Determining a combination score value by calculating the ratio of the expression of the 2 genes, wherein the ratio is calculated by:
i. Combining a first gene comprised in the tumor cell proliferation signature with a second gene comprised in the immune signature; or
ii. Combining a first gene comprised in the luminal differentiation signature with a second gene comprised in the immune signature; or
iii. Combining a first gene comprised in the tumor cell proliferation signature with a second gene comprised in the luminal differentiation signature; or
iv. Combining a first gene comprised in the immune signature selected from the group consisting of: CD27, CXCL8, HLA-C, IGLV3-25, IL2RG, LAX1, NTN3, PIM2 or POU2AF1 with a second gene comprised in the immune signature selected from the group consisting of: CD79A, CD27, IGJ, POU2AF1, TNFRSF17, IL2RG, PIM2 or IGL;
c. Wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EX01, ASPM, NEK2 or KIF23] and the luminal differentiation signature comprises the genes: [BCL2, DNAJC12, AGR3, AFF3 or ESR1]; and
d. Wherein if a deviation of the combination score value is identified, as compared with a pre-established reference value, is indicative of poor prognosis.

6. In vitro method for the prognosis of patients suffering from HER2+ breast cancer, according to claim 1, which comprises:
a. Measuring the level of expression of at least two genes selected from the gene combinations of Table 7B, in a biological sample obtained from the patient;
b. Determining a combination score value by calculating the ratio of the expression of the 2 genes; and
c. Wherein if a deviation of the combination score value is identified, as compared with a pre-established reference value, is indicative of poor prognosis.

7. In vitro method for the prognosis of patients suffering from HER2+ breast cancer, according to claim 1, which comprises measuring the level of expression of a group of genes consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and ESR1].

8. In vitro method for identifying biomarker signatures for the prediction of response to anti-HER2 therapies in patients suffering from HER2+ breast cancer, or for classifying patients into responder or non-responder patients to anti-HER2 therapies, which comprises:
a. Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP], in a biological sample obtained from the patient;
b. Determining a combination score value by calculating the ratio of the expression of the 2 genes; and
c. Wherein if a deviation of the combination score value is identified, as compared with a pre-established reference value, this is indicative that the biomarker signature may be used for the prediction of response to anti-HER2 therapies in patients suffering from HER2+ breast cancer, or for classifying patients into responder or non-responder patients to anti-HER2 therapies.

9. In vitro method for the prediction of response to anti-HER2 therapies in patients suffering from HER2+ breast cancer, or for classifying patients into responder or non-responder patients to anti-HER2 therapies, according to claim 8, which comprises:
a. Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP], in a biological sample obtained from the patient;
b. Determining a combination score value by calculating the ratio of the expression of the 2 genes; and
c. Wherein if a deviation of the combination score value is identified, as compared with a pre-established reference value, this is indicative of the response to anti-HER2 therapies in patients suffering from HER2+ breast cancer.

10. In vitro method for the prediction of response to anti-HER2 therapies in patients suffering from HER2+ breast cancer, or for classifying patients into responder or non-responder patients to anti-HER2 therapies, according to claim 8, which comprises:
a. Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP], in a biological sample obtained from the patient;
b. Determining a combination score value by calculating the ratio of the expression of the 2 genes, wherein the ratio is calculated by:
i. Combining a first gene comprised in the immune signature with a second gene comprised in the luminal differentiation signature; or
ii. Combining a first gene comprised in the tumor cell proliferation signature with a second gene comprised in the luminal differentiation signature; or
iii. Combining a first gene comprised in the HER2 amplicon signature with a second gene comprised in the immune signature; or
iv. Combining a first gene comprised in the HER2 amplicon signature with a second gene comprised in the tumor cell proliferation signature; or
v. Combining a first gene comprised in the HER2 amplicon signature with a second gene comprised in the luminal differentiation signature; or
vi. Combining a first gene comprised in the immune signature selected from the group consisting of: IGKC, IGL or LAX1 with a second gene comprised in the immune signature selected from the group consisting of: HLA-C, CD27, IGJ, LAX1, NTN3, PIM2, POU2AF1 or TNFRSF17; or
vii. Combining a first gene comprised in the luminal differentiation signature selected from the group consisting of: AFF3, BCL2 or DNAJC12, with a second gene comprised in the luminal differentiation signature selected from the group consisting of: ESR1 or AGR3; or
viii. Combining the first gene ASPM comprised in the tumor cell proliferation signature with the second gene NEK2 comprised in the tumor cell proliferation signature; and
c. Wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EX01, ASPM, NEK2 or KIF23], the luminal differentiation signature comprises the genes: [BCL2, DNAJC12, AGR3, AFF3 or ESR1] and the HER2 amplicon signature comprises the genes: [ERBB2, GRB7, STARD3 or TCAP], and
d. Wherein if a deviation of the combination score value is identified, as compared with a pre-established reference value, is an indication that the patients suffering from HER2+ breast cancer may respond to anti-HER2 therapies.

11. In vitro method for the prediction of response to anti-HER2 therapies in patients suffering from HER2+ breast cancer, or for classifying patients into responder or non-responder patients to anti-HER2 therapies, according to claim 8, which comprises:
a. Measuring the level of expression of at least two genes selected from the gene combinations of Table 9A, in a biological sample obtained from the patient;
b. Determining a combination score value by calculating the ratio of the expression of the 2 genes; and
c. Wherein if a deviation of the combination score value is identified, as compared with a pre-established reference value, is an indication that the patients suffering from HER2+ breast cancer may respond to anti-HER2 therapies.

12. In vitro method for the prediction of response to anti-HER2 therapies in patients suffering from HER2+ breast cancer, or for classifying patients into responder or non-responder patients to anti-HER2 therapies, according to claim 8, which comprises:
a. Measuring the level of expression of at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 or TCAP], in a biological sample obtained from the patient;
b. Determining a combination score value by calculating the ratio of the expression of the 2 genes, wherein the ratio is calculated by:
i. Combining a first gene comprised in the luminal differentiation signature with a second gene comprised in the immune signature; or
ii. Combining a first gene comprised in the luminal differentiation signature with a second gene comprised in the tumor cell proliferation signature; or
iii. Combining a first gene comprised in the immune differentiation signature with a second gene comprised in the HER2 amplicon signature; or
iv. Combining a first gene comprised in the tumor cell proliferation signature with a second gene comprised in the HER2 amplicon signature; or
v. Combining a first gene comprised in the luminal differentiation signature with a second gene comprised in the HER2 amplicon signature; or
vi. Combining a first gene comprised in the immune signature selected from the group consisting of: HLA-C, CD27, IGJ, LAX1, NTN3, PIM2, POU2AF1 or TNFRSF17 with a second gene comprised in the immune signature selected from the group consisting of: IGKC, IGL or LAX1; or
vii. Combining a first gene comprised in the luminal differentiation signature selected from the group consisting of: ESR1 or AGR3 with a second gene comprised in the luminal differentiation signature selected from the group consisting of: AFF3, BCL2, or DNAJC12; or
viii. Combining the first gene NEK2 comprised in the tumor cell proliferation signature with the second gene ASPM comprised in the tumor cell proliferation signature; and
c. Wherein the immune signature comprises the genes [CD27, CD79A, HLA-C, IGJ, IGKC, IGL, IGLV3-25, IL2RG, CXCL8, LAX1, NTN3, PIM2, POU2AF1 or TNFRSF17], the tumor cell proliferation signature comprises the genes [EX01, ASPM, NEK2 or KIF23], the luminal differentiation signature comprises the genes: [BCL2, DNAJC12, AGR3, AFF3 or ESR1] and the HER2 amplicon signature comprises the genes: [ERBB2, GRB7, STARD3 or TCAP], and
d. Wherein if a deviation of the combination score value is identified, as compared with a pre-established reference value, is an indication that the patients suffering from HER2+ breast cancer may not respond to anti-HER2 therapies.

13. In vitro method for the prediction of response to anti-HER2 therapies in patients suffering from HER2+ breast cancer, or for classifying patients into responder or non-responder patients to anti-HER2 therapies, according to claim 8, which comprises:
a. Measuring the level of expression of at least two genes selected from the gene combinations of Table 9B, in a biological sample obtained from the patient;
b. Determining a combination score value by calculating the ratio of the expression of the 2 genes; and
c. Wherein if a deviation of the combination score value is identified, as compared with a pre-established reference value, is an indication that the patients suffering from HER2+ breast cancer may not respond to anti-HER2 therapies.

14. In vitro method for the prediction of response to anti-HER2 therapies in patients suffering from HER2+ breast cancer, or for classifying patients into responder or non-responder patients to anti-HER2 therapies, according to claim 8, which comprises measuring the level of expression of a group of genes consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3, ESR1, ERBB2, GRB7, STARD3 and TCAP].

15. In vitro method, according to claim 1, which further comprises identifying the nodal status (pN1) and/or tumor staging (pT2-4) wherein the identification of nodal status N1-3 and/or tumor status T2-4 is indicative of bad prognosis or that the patient is a non-responder patient to anti-HER2 therapies.

16. (canceled)

17. In vitro method, according to claim 1, wherein the sample is selected form: tissue, blood, serum or plasma.

18. (canceled)

19. In vitro use at least two genes selected from the group consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 or ESR1] for identifying biomarker signatures for the prognosis of patients suffering from HER2+ breast cancer.

20-24. (canceled)

25. In vitro use of a group of genes consisting of: [CD27, CD79A, HLA-C, IGJ, IGKC, IGL, IGLV3-25, IL2RG, CXCL8, LAX1, NTN3, PIM2, POU2AF1, TNFRSF17, EXO1, ASPM, NEK2, KIF23, BCL2, DNAJC12, AGR3, AFF3 and ESR1], according to claim 19, for the prognosis of patients suffering from HER2+ breast cancer.

26-32. (canceled)

33. Anti-HER2 therapy, or any pharmaceutical composition comprising thereof, optionally including pharmaceutically acceptable excipients or carriers, for use in the treatment of patients suffering from HER2+ breast cancer, wherein the method comprises predicting the response to anti-HER2 therapies in the patients suffering from HER2+ breast cancer or classifying patients into responder or non-responder patients to anti-HER2 therapies, by following the method of claim 8.

34. Anti-HER2 therapy, or any pharmaceutical composition comprising thereof, optionally including pharmaceutically acceptable excipients or carriers, for use in the treatment of patients suffering from HER2+ breast cancer, according to claim 33, wherein the anti-HER2 therapy is optionally selected from: trastuzumab, pertuzumab, lapatinib, pyrotinib, poziotinib, tucatinib, neratinib, trastuzumab deruxtecan, SYD985 or ado-trastuzumab emtansine.
